SEQUENCE LISTING (629 sequences)

SEQ ID NO: 1 - protein, 18 aa, Artificial
  Note  Artificial = synthetic construct
GEEMRDRARAHVDALRTH

SEQ ID NO: 2 - protein, 18 aa, Artificial
  Note  Artificial = synthetic construct
HTRLADVHARARDRMEEG

SEQ ID NO: 3 - protein, 18 aa, Artificial
  Note  Artificial = synthetic construct
SDELRQRLAARLEALKEN

SEQ ID NO: 4 - protein, 18 aa, Artificial
  Note  Artificial = synthetic construct
NEKLAELRAALRQRLEDS

SEQ ID NO: 5 - protein, 18 aa, Artificial
  Note  Artificial = synthetic construct
LESAKVSALSALEEATKK

SEQ ID NO: 6 - protein, 18 aa, Artificial
  Note  Artificial = synthetic construct
KKTAEELASLASVKASEL

SEQ ID NO: 7 - protein, 5 aa, Artificial
  Note  Artificial = synthetic construct
KLSPL

SEQ ID NO: 8 - protein, 4 aa, Artificial
  Note  Artificial = synthetic construct
LSPL

SEQ ID NO: 9 - protein, 3 aa, Artificial
  Note  Artificial = synthetic construct
SPL

SEQ ID NO: 10 - protein, 5 aa, Artificial
  Note  Artificial = synthetic construct
LPSLK

SEQ ID NO: 11 - protein, 4 aa, Artificial
  Note  Artificial = synthetic construct
LPSL

SEQ ID NO: 12 - protein, 3 aa, Artificial
  Note  Artificial = synthetic construct
LPS

SEQ ID NO: 13 - protein, 18 aa, Artificial
  Note  Artificial = synthetic construct
LESFKVSFLSALEEYTKK

SEQ ID NO: 14 - protein, 18 aa, Artificial
  Note  Artificial = synthetic construct
KKTYEELASLFSVKFSEL

SEQ ID NO: 15 - protein, 9 aa, Artificial
  Note  Artificial = synthetic construct
PRGGSVLVT

SEQ ID NO: 16 - protein, 9 aa, Artificial
  Note  Artificial = synthetic construct
TVLVSGGRP

SEQ ID NO: 17 - protein, 6 aa, Artificial
  Note  Artificial = synthetic construct
PRGGSV

SEQ ID NO: 18 - protein, 6 aa, Artificial
  Note  Artificial = synthetic construct
TVLVSG

SEQ ID NO: 19 - protein, 5 aa, Artificial
  Note  Artificial = synthetic construct
ALSPL

SEQ ID NO: 20 - protein, 5 aa, Artificial
  Note  Artificial = synthetic construct
LPSLA

SEQ ID NO: 21 - protein, 6 aa, Artificial
  Note  Artificial = synthetic construct
WRWWWW

SEQ ID NO: 22 - protein, 6 aa, Artificial
  Note  Artificial = synthetic construct
WWWWRW

SEQ ID NO: 23 - protein, 4 aa, Artificial
  Note  Artificial = synthetic construct
LNTQ

SEQ ID NO: 24 - protein, 4 aa, Artificial
  Note  Artificial = synthetic construct
QTNL

SEQ ID NO: 25 - protein, 6 aa, Artificial
  Note  Artificial = synthetic construct
LKLSPL

SEQ ID NO: 26 - protein, 6 aa, Artificial
  Note  Artificial = synthetic construct
LPSLKL

SEQ ID NO: 27 - protein, 4 aa, Artificial
  Note  Artificial = synthetic construct
GPGG

SEQ ID NO: 28 - protein, 5 aa, Artificial
  Note  Artificial = synthetic construct
GGGGS

SEQ ID NO: 29 - protein, 4 aa, Artificial
  Note  Artificial = synthetic construct
GPGG

SEQ ID NO: 30 - protein, 8 aa, Artificial
  Note  Artificial = synthetic construct
GPGGGPGG

SEQ ID NO: 31 - protein, 12 aa, Artificial
  Note  Artificial = synthetic construct
GPGGGPGGGPGG

SEQ ID NO: 32 - protein, 16 aa, Artificial
  Note  Artificial = synthetic construct
GPGGGPGGGPGGGPGG

SEQ ID NO: 33 - protein, 5 aa, Artificial
  Note  Artificial = synthetic construct
GGGGS

SEQ ID NO: 34 - protein, 10 aa, Artificial
  Note  Artificial = synthetic construct
GGGGSGGGGS

SEQ ID NO: 35 - protein, 15 aa, Artificial
  Note  Artificial = synthetic construct
GGGGSGGGGSGGGGS

SEQ ID NO: 36 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 37 - protein, 41 aa, Artificial
  Note  Artificial = synthetic construct
GEEMRDRARAHVDALRTHKLSPLLESFKVSFLSALEEYTKK

SEQ ID NO: 38 - protein, 37 aa, Artificial
  Note  Artificial = synthetic construct
GEEMRDRARAHVDALRTHPLESFKVSFLSALEEYTKK

SEQ ID NO: 39 - protein, 41 aa, Artificial
  Note  Artificial = synthetic construct
SDELRQRLAARLEALKENKLSPLLESFKVSFLSALEEYTKK

SEQ ID NO: 40 - protein, 37 aa, Artificial
  Note  Artificial = synthetic construct
SDELRQRLAARLEALKENPLESFKVSFLSALEEYTKK

SEQ ID NO: 41 - protein, 41 aa, Artificial
  Note  Artificial = synthetic construct
LESAKVSALSALEEATKKKLSPLLESFKVSFLSALEEYTKK

SEQ ID NO: 42 - protein, 37 aa, Artificial
  Note  Artificial = synthetic construct
LESAKVSALSALEEATKKPLESFKVSFLSALEEYTKK

SEQ ID NO: 43 - protein, 41 aa, Artificial
  Note  Artificial = synthetic construct
LESAKVSALSALEEATKKKLSPLKKTYEELASLFSVKFSEL

SEQ ID NO: 44 - protein, 37 aa, Artificial
  Note  Artificial = synthetic construct
LESAKVSALSALEEATKKPKKTYEELASLFSVKFSEL

SEQ ID NO: 45 - protein, 41 aa, Artificial
  Note  Artificial = synthetic construct
KKTAEELASLASVKASELKLSPLKKTYEELASLFSVKFSEL

SEQ ID NO: 46 - protein, 37 aa, Artificial
  Note  Artificial = synthetic construct
KKTAEELASLASVKASELPKKTYEELASLFSVKFSEL

SEQ ID NO: 47 - protein, 41 aa, Artificial
  Note  Artificial = synthetic construct
KKTAEELASLASVKASELKLSPLLESFKVSFLSALEEYTKK

SEQ ID NO: 48 - protein, 37 aa, Artificial
  Note  Artificial = synthetic construct
KKTAEELASLASVKASELPLESFKVSFLSALEEYTKK

SEQ ID NO: 49 - protein, 41 aa, Artificial
  Note  Artificial = synthetic construct
LESAKVSALSALEEATKKLPSLKLESFKVSFLSALEEYTKK

SEQ ID NO: 50 - protein, 37 aa, Artificial
  Note  Artificial = synthetic construct
LESAKVSALSALEEATKPKLESFKVSFLSALEEYTKK

SEQ ID NO: 51 - protein, 41 aa, Artificial
  Note  Artificial = synthetic construct
LESAKVSALSALEEATKKLPSLKKKTYEELASLFSVKFSEL

SEQ ID NO: 52 - protein, 37 aa, Artificial
  Note  Artificial = synthetic construct
LESAKVSALSALEEATKPKKKTYEELASLFSVKFSEL

SEQ ID NO: 53 - protein, 41 aa, Artificial
  Note  Artificial = synthetic construct
KKTAEELASLASVKASELLPSLKKKTYEELASLFSVKFSEL

SEQ ID NO: 54 - protein, 41 aa, Artificial
  Note  Artificial = synthetic construct
KKTAEELASLASVKASELLPSLKLESFKVSFLSALEEYTKK

SEQ ID NO: 55 - protein, 36 aa, Artificial
  Note  Artificial = synthetic construct
KKTAEELASLASVKASELPESFKVSFLSALEEYTKK

SEQ ID NO: 56 - protein, 41 aa, Artificial
  Note  Artificial = synthetic construct
LESFLVSFKSALEEYFEKKLSPLLESAAVSAKSALEEYAEK

SEQ ID NO: 57 - protein, 66 aa, Artificial
  Note  Artificial = synthetic construct
PRGGSVLVTLPSLKGEEMRDRARAHVDALRTHKLSPLLESFKVSFLSALEEYTKKALSPLWRWWWW

SEQ ID NO: 58 - protein, 55 aa, Artificial
  Note  Artificial = synthetic construct
PRGGSVLVTLPSLKGEEMRDRARAHVDALRTHKLSPLLESFKVSFLSALEEYTKK

SEQ ID NO: 59 - protein, 52 aa, Artificial
  Note  Artificial = synthetic construct
GEEMRDRARAHVDALRTHKLSPLLESFKVSFLSALEEYTKKALSPLWRWWWW

SEQ ID NO: 60 - protein, 66 aa, Artificial
  Note  Artificial = synthetic construct
PRGGSVLVTLPSLKSDELRQRLAARLEALKENKLSPLLESFKVSFLSALEEYTKKALSPLWRWWWW

SEQ ID NO: 61 - protein, 55 aa, Artificial
  Note  Artificial = synthetic construct
PRGGSVLVTLPSLKSDELRQRLAARLEALKENKLSPLLESFKVSFLSALEEYTKK

SEQ ID NO: 62 - protein, 52 aa, Artificial
  Note  Artificial = synthetic construct
SDELRQRLAARLEALKENKLSPLLESFKVSFLSALEEYTKKALSPLWRWWWW

SEQ ID NO: 63 - protein, 62 aa, Artificial
  Note  Artificial = synthetic construct
PRGGSVLVTLPSLKGEEMRDRARAHVDALRTHPLESFKVSFLSALEEYTKKALSPLWRWWWW

SEQ ID NO: 64 - protein, 51 aa, Artificial
  Note  Artificial = synthetic construct
PRGGSVLVTLPSLKGEEMRDRARAHVDALRTHPLESFKVSFLSALEEYTKK

SEQ ID NO: 65 - protein, 48 aa, Artificial
  Note  Artificial = synthetic construct
GEEMRDRARAHVDALRTHPLESFKVSFLSALEEYTKKALSPLWRWWWW

SEQ ID NO: 66 - protein, 62 aa, Artificial
  Note  Artificial = synthetic construct
PRGGSVLVTLPSLKSDELRQRLAARLEALKENPLESFKVSFLSALEEYTKKALSPLWRWWWW

SEQ ID NO: 67 - protein, 51 aa, Artificial
  Note  Artificial = synthetic construct
PRGGSVLVTLPSLKSDELRQRLAARLEALKENPLESFKVSFLSALEEYTKK

SEQ ID NO: 68 - protein, 48 aa, Artificial
  Note  Artificial = synthetic construct
SDELRQRLAARLEALKENPLESFKVSFLSALEEYTKKALSPLWRWWWW

SEQ ID NO: 69 - protein, 66 aa, Artificial
  Note  Artificial = synthetic construct
PRGGSVLVTLPSLKLESAKVSALSALEEATKKKLSPLLESFKVSFLSALEEYTKKALSPLWRWWWW

SEQ ID NO: 70 - protein, 55 aa, Artificial
  Note  Artificial = synthetic construct
PRGGSVLVTLPSLKLESAKVSALSALEEATKKKLSPLLESFKVSFLSALEEYTKK

SEQ ID NO: 71 - protein, 52 aa, Artificial
  Note  Artificial = synthetic construct
LESAKVSALSALEEATKKKLSPLLESFKVSFLSALEEYTKKALSPLWRWWWW

SEQ ID NO: 72 - protein, 66 aa, Artificial
  Note  Artificial = synthetic construct
PRGGSVLVTLPSLKKKTAEELASLASVKASELKLSPLLESFKVSFLSALEEYTKKALSPLWRWWWW

SEQ ID NO: 73 - protein, 55 aa, Artificial
  Note  Artificial = synthetic construct
PRGGSVLVTLPSLKKKTAEELASLASVKASELKLSPLLESFKVSFLSALEEYTKK

SEQ ID NO: 74 - protein, 52 aa, Artificial
  Note  Artificial = synthetic construct
KKTAEELASLASVKASELKLSPLLESFKVSFLSALEEYTKKALSPLWRWWWW

SEQ ID NO: 75 - protein, 66 aa, Artificial
  Note  Artificial = synthetic construct
PRGGSVLVTLPSLKKKTAEELASLASVKASELKLSPLKKTYEELASLFSVKFSELALSPLWRWWWW

SEQ ID NO: 76 - protein, 55 aa, Artificial
  Note  Artificial = synthetic construct
PRGGSVLVTLPSLKKKTAEELASLASVKASELKLSPLKKTYEELASLFSVKFSEL

SEQ ID NO: 77 - protein, 47 aa, Artificial
  Note  Artificial = synthetic construct
KKTAEELASLASVKASELKLSPLKKTYEELASLFSVKFSELWRWWWW

SEQ ID NO: 78 - protein, 62 aa, Artificial
  Note  Artificial = synthetic construct
PRGGSVLVTLPSLKLESAKVSALSALEEATKKPLESFKVSFLSALEEYTKKALSPLWRWWWW

SEQ ID NO: 79 - protein, 51 aa, Artificial
  Note  Artificial = synthetic construct
PRGGSVLVTLPSLKLESAKVSALSALEEATKKPLESFKVSFLSALEEYTKK

SEQ ID NO: 80 - protein, 48 aa, Artificial
  Note  Artificial = synthetic construct
LESAKVSALSALEEATKKPLESFKVSFLSALEEYTKKALSPLWRWWWW

SEQ ID NO: 81 - protein, 62 aa, Artificial
  Note  Artificial = synthetic construct
PRGGSVLVTLPSLKKKTAEELASLASVKASELPLESFKVSFLSALEEYTKKALSPLWRWWWW

SEQ ID NO: 82 - protein, 51 aa, Artificial
  Note  Artificial = synthetic construct
PRGGSVLVTLPSLKKKTAEELASLASVKASELPLESFKVSFLSALEEYTKK

SEQ ID NO: 83 - protein, 48 aa, Artificial
  Note  Artificial = synthetic construct
KKTAEELASLASVKASELPLESFKVSFLSALEEYTKKALSPLWRWWWW

SEQ ID NO: 84 - protein, 62 aa, Artificial
  Note  Artificial = synthetic construct
PRGGSVLVTLPSLKKKTAEELASLASVKASELKPKTYEELASLFSVKFSELALSPLWRWWWW

SEQ ID NO: 85 - protein, 51 aa, Artificial
  Note  Artificial = synthetic construct
PRGGSVLVTLPSLKKKTAEELASLASVKASELPKKTYEELASLFSVKFSEL

SEQ ID NO: 86 - protein, 43 aa, Artificial
  Note  Artificial = synthetic construct
KKTAEELASLASVKASELPKKTYEELASLFSVKFSELWRWWWW

SEQ ID NO: 87 - protein, 62 aa, Artificial
  Note  Artificial = synthetic construct
PRGGSVLVTLPSLKKKTAEELASLASVKASELPKKTYEELASLFSVKFSELALSPLWRWWWW

SEQ ID NO: 88 - protein, 66 aa, Artificial
  Note  Artificial = synthetic construct
WRWWWWLPSLKGEEMRDRARAHVDALRTHKLSPLLESFKVSFLSALEEYTKKALSPLPRGGSVLVT

SEQ ID NO: 89 - protein, 55 aa, Artificial
  Note  Artificial = synthetic construct
GEEMRDRARAHVDALRTHKLSPLLESFKVSFLSALEEYTKKALSPLPRGGSVLVT

SEQ ID NO: 90 - protein, 52 aa, Artificial
  Note  Artificial = synthetic construct
WRWWWWLPSLKGEEMRDRARAHVDALRTHKLSPLLESFKVSFLSALEEYTKK

SEQ ID NO: 91 - protein, 66 aa, Artificial
  Note  Artificial = synthetic construct
WRWWWWLPSLKSDELRQRLAARLEALKENKLSPLLESFKVSFLSALEEYTKKALSPLPRGGSVLVT

SEQ ID NO: 92 - protein, 55 aa, Artificial
  Note  Artificial = synthetic construct
SDELRQRLAARLEALKENKLSPLLESFKVSFLSALEEYTKKALSPLPRGGSVLVT

SEQ ID NO: 93 - protein, 52 aa, Artificial
  Note  Artificial = synthetic construct
WRWWWWLPSLKSDELRQRLAARLEALKENKLSPLLESFKVSFLSALEEYTKK

SEQ ID NO: 94 - protein, 62 aa, Artificial
  Note  Artificial = synthetic construct
WRWWWWLPSLKGEEMRDRARAHVDALRTHPLESFKVSFLSALEEYTKKALSPLPRGGSVLVT

SEQ ID NO: 95 - protein, 51 aa, Artificial
  Note  Artificial = synthetic construct
GEEMRDRARAHVDALRTHPLESFKVSFLSALEEYTKKALSPLPRGGSVLVT

SEQ ID NO: 96 - protein, 48 aa, Artificial
  Note  Artificial = synthetic construct
WRWWWWLPSLKGEEMRDRARAHVDALRTHPLESFKVSFLSALEEYTKK

SEQ ID NO: 97 - protein, 62 aa, Artificial
  Note  Artificial = synthetic construct
WRWWWWLPSLKSDELRQRLAARLEALKENPLESFKVSFLSALEEYTKKALSPLPRGGSVLVT

SEQ ID NO: 98 - protein, 51 aa, Artificial
  Note  Artificial = synthetic construct
SDELRQRLAARLEALKENPLESFKVSFLSALEEYTKKALSPLPRGGSVLVT

SEQ ID NO: 99 - protein, 48 aa, Artificial
  Note  Artificial = synthetic construct
WRWWWWLPSLKSDELRQRLAARLEALKENPLESFKVSFLSALEEYTKK

SEQ ID NO: 100 - protein, 66 aa, Artificial
  Note  Artificial = synthetic construct
WRWWWWLPSLKLESAKVSALSALEEATKKKLSPLLESFKVSFLSALEEYTKKALSPLPRGGSVLVT

SEQ ID NO: 101 - protein, 55 aa, Artificial
  Note  Artificial = synthetic construct
LESAKVSALSALEEATKKKLSPLLESFKVSFLSALEEYTKKALSPLPRGGSVLVT

SEQ ID NO: 102 - protein, 52 aa, Artificial
  Note  Artificial = synthetic construct
WRWWWWLPSLKLESAKVSALSALEEATKKKLSPLLESFKVSFLSALEEYTKK

SEQ ID NO: 103 - protein, 66 aa, Artificial
  Note  Artificial = synthetic construct
WRWWWWLPSLKKKTAEELASLASVKASELKLSPLLESFKVSFLSALEEYTKKALSPLPRGGSVLVT

SEQ ID NO: 104 - protein, 55 aa, Artificial
  Note  Artificial = synthetic construct
KKTAEELASLASVKASELKLSPLLESFKVSFLSALEEYTKKALSPLPRGGSVLVT

SEQ ID NO: 105 - protein, 52 aa, Artificial
  Note  Artificial = synthetic construct
WRWWWWLPSLKKKTAEELASLASVKASELKLSPLLESFKVSFLSALEEYTKK

SEQ ID NO: 106 - protein, 66 aa, Artificial
  Note  Artificial = synthetic construct
WRWWWWLPSLKKKTAEELASLASVKASELKLSPLKKTYEELASLFSVKFSELALSPLPRGGSVLVT

SEQ ID NO: 107 - protein, 55 aa, Artificial
  Note  Artificial = synthetic construct
KKTAEELASLASVKASELKLSPLKKTYEELASLFSVKFSELALSPLPRGGSVLVT

SEQ ID NO: 108 - protein, 52 aa, Artificial
  Note  Artificial = synthetic construct
WRWWWWLPSLKKKTAEELASLASVKASELKLSPLKKTYEELASLFSVKFSEL

SEQ ID NO: 109 - protein, 62 aa, Artificial
  Note  Artificial = synthetic construct
WRWWWWLPSLKLESAKVSALSALEEATKKPLESFKVSFLSALEEYTKKALSPLPRGGSVLVT

SEQ ID NO: 110 - protein, 51 aa, Artificial
  Note  Artificial = synthetic construct
LESAKVSALSALEEATKKPLESFKVSFLSALEEYTKKALSPLPRGGSVLVT

SEQ ID NO: 111 - protein, 48 aa, Artificial
  Note  Artificial = synthetic construct
WRWWWWLPSLKLESAKVSALSALEEATKKPLESFKVSFLSALEEYTKK

SEQ ID NO: 112 - protein, 62 aa, Artificial
  Note  Artificial = synthetic construct
WRWWWWLPSLKKKTAEELASLASVKASELPLESFKVSFLSALEEYTKKALSPLPRGGSVLVT

SEQ ID NO: 113 - protein, 51 aa, Artificial
  Note  Artificial = synthetic construct
KKTAEELASLASVKASELPLESFKVSFLSALEEYTKKALSPLPRGGSVLVT

SEQ ID NO: 114 - protein, 48 aa, Artificial
  Note  Artificial = synthetic construct
WRWWWWLPSLKKKTAEELASLASVKASELPLESFKVSFLSALEEYTKK

SEQ ID NO: 115 - protein, 62 aa, Artificial
  Note  Artificial = synthetic construct
WRWWWWLPSLKKKTAEELASLASVKASELPKKTYEELASLFSVKFSELALSPLPRGGSVLVT

SEQ ID NO: 116 - protein, 51 aa, Artificial
  Note  Artificial = synthetic construct
KKTAEELASLASVKASELPKKTYEELASLFSVKFSELALSPLPRGGSVLVT

SEQ ID NO: 117 - protein, 48 aa, Artificial
  Note  Artificial = synthetic construct
WRWWWWLPSLKKKTAEELASLASVKASELPKKTYEELASLFSVKFSEL

SEQ ID NO: 118 - protein, 48 aa, Artificial
  Note  Artificial = synthetic construct
SPLGEEMRDRARAHVDALRTHKLSPLLESFKVSFLSALEEYTKKLNTQ

SEQ ID NO: 119 - protein, 45 aa, Artificial
  Note  Artificial = synthetic construct
GEEMRDRARAHVDALRTHKLSPLLESFKVSFLSALEEYTKKLNTQ

SEQ ID NO: 120 - protein, 44 aa, Artificial
  Note  Artificial = synthetic construct
SPLGEEMRDRARAHVDALRTHKLSPLLESFKVSFLSALEEYTKK

SEQ ID NO: 121 - protein, 48 aa, Artificial
  Note  Artificial = synthetic construct
SPLSDELRQRLAARLEALKENKLSPLLESFKVSFLSALEEYTKKLNTQ

SEQ ID NO: 122 - protein, 45 aa, Artificial
  Note  Artificial = synthetic construct
SDELRQRLAARLEALKENKLSPLLESFKVSFLSALEEYTKKLNTQ

SEQ ID NO: 123 - protein, 44 aa, Artificial
  Note  Artificial = synthetic construct
SPLSDELRQRLAARLEALKENKLSPLLESFKVSFLSALEEYTKK

SEQ ID NO: 124 - protein, 44 aa, Artificial
  Note  Artificial = synthetic construct
SPLGEEMRDRARAHVDALRTHPLESFKVSFLSALEEYTKKLNTQ

SEQ ID NO: 125 - protein, 41 aa, Artificial
  Note  Artificial = synthetic construct
GEEMRDRARAHVDALRTHPLESFKVSFLSALEEYTKKLNTQ

SEQ ID NO: 126 - protein, 40 aa, Artificial
  Note  Artificial = synthetic construct
SPLGEEMRDRARAHVDALRTHPLESFKVSFLSALEEYTKK

SEQ ID NO: 127 - protein, 44 aa, Artificial
  Note  Artificial = synthetic construct
SPLSDELRQRLAARLEALKENPLESFKVSFLSALEEYTKKLNTQ

SEQ ID NO: 128 - protein, 41 aa, Artificial
  Note  Artificial = synthetic construct
SDELRQRLAARLEALKENPLESFKVSFLSALEEYTKKLNTQ

SEQ ID NO: 129 - protein, 40 aa, Artificial
  Note  Artificial = synthetic construct
SPLSDELRQRLAARLEALKENPLESFKVSFLSALEEYTKK

SEQ ID NO: 130 - protein, 48 aa, Artificial
  Note  Artificial = synthetic construct
SPLLESAKVSALSALEEATKKKLSPLLESFKVSFLSALEEYTKKLNTQ

SEQ ID NO: 131 - protein, 45 aa, Artificial
  Note  Artificial = synthetic construct
LESAKVSALSALEEATKKKLSPLLESFKVSFLSALEEYTKKLNTQ

SEQ ID NO: 132 - protein, 44 aa, Artificial
  Note  Artificial = synthetic construct
SPLLESAKVSALSALEEATKKKLSPLLESFKVSFLSALEEYTKK

SEQ ID NO: 133 - protein, 48 aa, Artificial
  Note  Artificial = synthetic construct
SPLKKTAEELASLASVKASELKLSPLLESFKVSFLSALEEYTKKLNTQ

SEQ ID NO: 134 - protein, 45 aa, Artificial
  Note  Artificial = synthetic construct
KKTAEELASLASVKASELKLSPLLESFKVSFLSALEEYTKKLNTQ

SEQ ID NO: 135 - protein, 44 aa, Artificial
  Note  Artificial = synthetic construct
SPLKKTAEELASLASVKASELKLSPLLESFKVSFLSALEEYTKK

SEQ ID NO: 136 - protein, 48 aa, Artificial
  Note  Artificial = synthetic construct
SPLKKTAEELASLASVKASELKLSPLKKTYEELASLFSVKFSELLNTQ

SEQ ID NO: 137 - protein, 45 aa, Artificial
  Note  Artificial = synthetic construct
KKTAEELASLASVKASELKLSPLKKTYEELASLFSVKFSELLNTQ

SEQ ID NO: 138 - protein, 44 aa, Artificial
  Note  Artificial = synthetic construct
SPLKKTAEELASLASVKASELKLSPLKKTYEELASLFSVKFSEL

SEQ ID NO: 139 - protein, 48 aa, Artificial
  Note  Artificial = synthetic construct
SPLLESAKVSALSALEEATKKKLSPLKKTYEELASLFSVKFSELLNTQ

SEQ ID NO: 140 - protein, 45 aa, Artificial
  Note  Artificial = synthetic construct
LESAKVSALSALEEATKKKLSPLKKTYEELASLFSVKFSELLNTQ

SEQ ID NO: 141 - protein, 44 aa, Artificial
  Note  Artificial = synthetic construct
SPLLESAKVSALSALEEATKKKLSPLKKTYEELASLFSVKFSEL

SEQ ID NO: 142 - protein, 44 aa, Artificial
  Note  Artificial = synthetic construct
SPLLESAKVSALSALEEATKKPLESFKVSFLSALEEYTKKLNTQ

SEQ ID NO: 143 - protein, 41 aa, Artificial
  Note  Artificial = synthetic construct
LESAKVSALSALEEATKKPLESFKVSFLSALEEYTKKLNTQ

SEQ ID NO: 144 - protein, 40 aa, Artificial
  Note  Artificial = synthetic construct
SPLLESAKVSALSALEEATKKPLESFKVSFLSALEEYTKK

SEQ ID NO: 145 - protein, 44 aa, Artificial
  Note  Artificial = synthetic construct
SPLKKTAEELASLASVKASELPLESFKVSFLSALEEYTKKLNTQ

SEQ ID NO: 146 - protein, 41 aa, Artificial
  Note  Artificial = synthetic construct
KKTAEELASLASVKASELPLESFKVSFLSALEEYTKKLNTQ

SEQ ID NO: 147 - protein, 40 aa, Artificial
  Note  Artificial = synthetic construct
SPLKKTAEELASLASVKASELPLESFKVSFLSALEEYTKK

SEQ ID NO: 148 - protein, 44 aa, Artificial
  Note  Artificial = synthetic construct
SPLKKTAEELASLASVKASELPKKTYEELASLFSVKFSELLNTQ

SEQ ID NO: 149 - protein, 41 aa, Artificial
  Note  Artificial = synthetic construct
KKTAEELASLASVKASELPKKTYEELASLFSVKFSELLNTQ

SEQ ID NO: 150 - protein, 40 aa, Artificial
  Note  Artificial = synthetic construct
SPLKKTAEELASLASVKASELPKKTYEELASLFSVKFSEL

SEQ ID NO: 151 - protein, 44 aa, Artificial
  Note  Artificial = synthetic construct
SPLLESAKVSALSALEEATKKPKKTYEELASLFSVKFSELLNTQ

SEQ ID NO: 152 - protein, 41 aa, Artificial
  Note  Artificial = synthetic construct
LESAKVSALSALEEATKKPKKTYEELASLFSVKFSELLNTQ

SEQ ID NO: 153 - protein, 40 aa, Artificial
  Note  Artificial = synthetic construct
SPLLESAKVSALSALEEATKKPKKTYEELASLFSVKFSEL

SEQ ID NO: 154 - protein, 59 aa, Artificial
  Note  Artificial = synthetic construct
PRGGSVLVTLPSLKGEEMRDRARAHVDALRTHKLSPLLESFKVSFLSALEEYTKKLNTQ

SEQ ID NO: 155 - protein, 55 aa, Artificial
  Note  Artificial = synthetic construct
SPLGEEMRDRARAHVDALRTHKLSPLLESFKVSFLSALEEYTKKALSPLWRWWWW

SEQ ID NO: 156 - protein, 59 aa, Artificial
  Note  Artificial = synthetic construct
PRGGSVLVTLPSLKSDELRQRLAARLEALKENKLSPLLESFKVSFLSALEEYTKKLNTQ

SEQ ID NO: 157 - protein, 55 aa, Artificial
  Note  Artificial = synthetic construct
SPLSDELRQRLAARLEALKENKLSPLLESFKVSFLSALEEYTKKALSPLWRWWWW

SEQ ID NO: 158 - protein, 55 aa, Artificial
  Note  Artificial = synthetic construct
PRGGSVLVTLPSLKGEEMRDRARAHVDALRTHPLESFKVSFLSALEEYTKKLNTQ

SEQ ID NO: 159 - protein, 51 aa, Artificial
  Note  Artificial = synthetic construct
SPLGEEMRDRARAHVDALRTHPLESFKVSFLSALEEYTKKALSPLWRWWWW

SEQ ID NO: 160 - protein, 55 aa, Artificial
  Note  Artificial = synthetic construct
PRGGSVLVTLPSLKSDELRQRLAARLEALKENPLESFKVSFLSALEEYTKKLNTQ

SEQ ID NO: 161 - protein, 51 aa, Artificial
  Note  Artificial = synthetic construct
SPLSDELRQRLAARLEALKENPLESFKVSFLSALEEYTKKALSPLWRWWWW

SEQ ID NO: 162 - protein, 59 aa, Artificial
  Note  Artificial = synthetic construct
PRGGSVLVTLPSLKLESAKVSALSALEEATKKKLSPLLESFKVSFLSALEEYTKKLNTQ

SEQ ID NO: 163 - protein, 55 aa, Artificial
  Note  Artificial = synthetic construct
SPLLESAKVSALSALEEATKKKLSPLLESFKVSFLSALEEYTKKALSPLWRWWWW

SEQ ID NO: 164 - protein, 59 aa, Artificial
  Note  Artificial = synthetic construct
PRGGSVLVTLPSLKKKTAEELASLASVKASELKLSPLLESFKVSFLSALEEYTKKLNTQ

SEQ ID NO: 165 - protein, 55 aa, Artificial
  Note  Artificial = synthetic construct
SPLKKTAEELASLASVKASELKLSPLLESFKVSFLSALEEYTKKALSPLWRWWWW

SEQ ID NO: 166 - protein, 59 aa, Artificial
  Note  Artificial = synthetic construct
PRGGSVLVTLPSLKKKTAEELASLASVKASELKLSPLKKTYEELASLFSVKFSELLNTQ

SEQ ID NO: 167 - protein, 55 aa, Artificial
  Note  Artificial = synthetic construct
SPLKKTAEELASLASVKASELKLSPLKKTYEELASLFSVKFSELALSPLWRWWWW

SEQ ID NO: 168 - protein, 59 aa, Artificial
  Note  Artificial = synthetic construct
PRGGSVLVTLPSLKLESAKVSALSALEEATKKKLSPLKKTYEELASLFSVKFSELLNTQ

SEQ ID NO: 169 - protein, 55 aa, Artificial
  Note  Artificial = synthetic construct
SPLLESAKVSALSALEEATKKKLSPLKKTYEELASLFSVKFSELALSPLWRWWWW

SEQ ID NO: 170 - protein, 55 aa, Artificial
  Note  Artificial = synthetic construct
PRGGSVLVTLPSLKLESAKVSALSALEEATKKPLESFKVSFLSALEEYTKKLNTQ

SEQ ID NO: 171 - protein, 51 aa, Artificial
  Note  Artificial = synthetic construct
SPLLESAKVSALSALEEATKKPLESFKVSFLSALEEYTKKALSPLWRWWWW

SEQ ID NO: 172 - protein, 55 aa, Artificial
  Note  Artificial = synthetic construct
PRGGSVLVTLPSLKKKTAEELASLASVKASELPLESFKVSFLSALEEYTKKLNTQ

SEQ ID NO: 173 - protein, 51 aa, Artificial
  Note  Artificial = synthetic construct
SPLKKTAEELASLASVKASELPLESFKVSFLSALEEYTKKALSPLWRWWWW

SEQ ID NO: 174 - protein, 55 aa, Artificial
  Note  Artificial = synthetic construct
PRGGSVLVTLPSLKKKTAEELASLASVKASELPKKTYEELASLFSVKFSELLNTQ

SEQ ID NO: 175 - protein, 51 aa, Artificial
  Note  Artificial = synthetic construct
SPLKKTAEELASLASVKASELPKKTYEELASLFSVKFSELALSPLWRWWWW

SEQ ID NO: 176 - protein, 55 aa, Artificial
  Note  Artificial = synthetic construct
PRGGSVLVTLPSLKLESAKVSALSALEEATKKPKKTYEELASLFSVKFSELLNTQ

SEQ ID NO: 177 - protein, 51 aa, Artificial
  Note  Artificial = synthetic construct
SPLLESAKVSALSALEEATKKPKKTYEELASLFSVKFSELALSPLWRWWWW

SEQ ID NO: 178 - protein, 21 aa, Artificial
  Note  Artificial = synthetic construct
PRGGSVLVTLKLSPLWRWWWW

SEQ ID NO: 179 - protein, 21 aa, Artificial
  Note  Artificial = synthetic construct
WRWWWWLKLSPLPRGGSVLVT

SEQ ID NO: 180 - protein, 17 aa, Artificial
  Note  Artificial = synthetic construct
PRGGSVLVTXPWRWWWW

SEQ ID NO: 181 - protein, 18 aa, Artificial
  Note  Artificial = synthetic construct
PRGGSVLVTXXPWRWWWW

SEQ ID NO: 182 - protein, 19 aa, Artificial
  Note  Artificial = synthetic construct
PRGGSVLVTXXXPWRWWWW

SEQ ID NO: 183 - protein, 20 aa, Artificial
  Note  Artificial = synthetic construct
PRGGSVLVTXXXXPWRWWWW

SEQ ID NO: 184 - protein, 16 aa, Artificial
  Note  Artificial = synthetic construct
PRGGSVLVTPWRWWWW

SEQ ID NO: 185 - protein, 17 aa, Artificial
  Note  Artificial = synthetic construct
XRGGSVLVTXPWRWWWW

SEQ ID NO: 186 - protein, 17 aa, Artificial
  Note  Artificial = synthetic construct
PXGGSVLVTXPWRWWWW

SEQ ID NO: 187 - protein, 17 aa, Artificial
  Note  Artificial = synthetic construct
PRXGSVLVTXPWRWWWW

SEQ ID NO: 188 - protein, 17 aa, Artificial
  Note  Artificial = synthetic construct
PRGXSVLVTXPWRWWWW

SEQ ID NO: 189 - protein, 17 aa, Artificial
  Note  Artificial = synthetic construct
PRGGXVLVTXPWRWWWW

SEQ ID NO: 190 - protein, 17 aa, Artificial
  Note  Artificial = synthetic construct
PRGGSXLVTXPWRWWWW

SEQ ID NO: 191 - protein, 16 aa, Artificial
  Note  Artificial = synthetic construct
XRGGSVLVTPWRWWWW

SEQ ID NO: 192 - protein, 16 aa, Artificial
  Note  Artificial = synthetic construct
PXGGSVLVTPWRWWWW

SEQ ID NO: 193 - protein, 16 aa, Artificial
  Note  Artificial = synthetic construct
PRXGSVLVTPWRWWWW

SEQ ID NO: 194 - protein, 16 aa, Artificial
  Note  Artificial = synthetic construct
PRGXSVLVTPWRWWWW

SEQ ID NO: 195 - protein, 16 aa, Artificial
  Note  Artificial = synthetic construct
PRGGXVLVTPWRWWWW

SEQ ID NO: 196 - protein, 16 aa, Artificial
  Note  Artificial = synthetic construct
PRGGSXLVTPWRWWWW

SEQ ID NO: 197 - protein, 17 aa, Artificial
  Note  Artificial = synthetic construct
XRGGSVLVTXPWRWWWX

SEQ ID NO: 198 - protein, 16 aa, Artificial
  Note  Artificial = synthetic construct
PRGGSVLVTPWWWWRW

SEQ ID NO: 199 - protein, 16 aa, Artificial
  Note  Artificial = synthetic construct
XXXXXXXXXXXXXXXX

SEQ ID NO: 200 - protein, 16 aa, Artificial
  Note  Artificial = synthetic construct
XRGGSVLVTPWWWWRX

SEQ ID NO: 201 - protein, 17 aa, Artificial
  Note  Artificial = synthetic construct
PRGGSVLVTXPWWWWRW

SEQ ID NO: 202 - protein, 17 aa, Artificial
  Note  Artificial = synthetic construct
XRGGSVLVTXPWWWWRX

SEQ ID NO: 203 - protein, 17 aa, Artificial
  Note  Artificial = synthetic construct
XXXXXXXXXXXXXXXXX

SEQ ID NO: 204 - protein, 18 aa, Artificial
  Note  Artificial = synthetic construct
DWLKAFYDKVAEKLKEAF

SEQ ID NO: 205 - protein, 18 aa, Artificial
  Note  Artificial = synthetic construct
DWAKAAYDKAAEKAKEAA

SEQ ID NO: 206 - protein, 37 aa, Artificial
  Note  Artificial = synthetic construct
DWLKAFYDKVAEKLKEAFPDWAKAAYDKAAEKAKEAA

SEQ ID NO: 207 - protein, 6 aa, Artificial
  Note  Artificial = synthetic construct
KAKEAA

SEQ ID NO: 208 - protein, 36 aa, Artificial
  Note  Artificial = synthetic construct
DWLKAFYDKVAEKLKEAPDWAKAAYDKAAEKAKEAA

SEQ ID NO: 209 - protein, 35 aa, Artificial
  Note  Artificial = synthetic construct
DWLKAFYDKVAEKLKEPDWAKAAYDKAAEKAKEAA

SEQ ID NO: 210 - protein, 34 aa, Artificial
  Note  Artificial = synthetic construct
DWLKAFYDKVAEKLKPDWAKAAYDKAAEKAKEAA

SEQ ID NO: 211 - protein, 33 aa, Artificial
  Note  Artificial = synthetic construct
DWLKAFYDKVAEKLPDWAKAAYDKAAEKAKEAA

SEQ ID NO: 212 - protein, 32 aa, Artificial
  Note  Artificial = synthetic construct
DWLKAFYDKVAEKPDWAKAAYDKAAEKAKEAA

SEQ ID NO: 213 - protein, 31 aa, Artificial
  Note  Artificial = synthetic construct
DWLKAFYDKVAEPDWAKAAYDKAAEKAKEAA

SEQ ID NO: 214 - protein, 36 aa, Artificial
  Note  Artificial = synthetic construct
DWLKAFYDKVAEKLKEAFPDWAKAAYDKAAEKAKEA

SEQ ID NO: 215 - protein, 35 aa, Artificial
  Note  Artificial = synthetic construct
DWLKAFYDKVAEKLKEAFPDWAKAAYDKAAEKAKE

SEQ ID NO: 216 - protein, 34 aa, Artificial
  Note  Artificial = synthetic construct
DWLKAFYDKVAEKLKEAFPDWAKAAYDKAAEKAK

SEQ ID NO: 217 - protein, 33 aa, Artificial
  Note  Artificial = synthetic construct
DWLKAFYDKVAEKLKEAFPDWAKAAYDKAAEKA

SEQ ID NO: 218 - protein, 32 aa, Artificial
  Note  Artificial = synthetic construct
DWLKAFYDKVAEKLKEAFPDWAKAAYDKAAEK

SEQ ID NO: 219 - protein, 31 aa, Artificial
  Note  Artificial = synthetic construct
DWLKAFYDKVAEKLKEAFPDWAKAAYDKAAE

SEQ ID NO: 220 - protein, 30 aa, Artificial
  Note  Artificial = synthetic construct
DWLKAFYDKVAEKLKEAFPDWAKAAYDKAA

SEQ ID NO: 221 - protein, 36 aa, Artificial
  Note  Artificial = synthetic construct
WLKAFYDKVAEKLKEAFPDWAKAAYDKAAEKAKEAA

SEQ ID NO: 222 - protein, 35 aa, Artificial
  Note  Artificial = synthetic construct
LKAFYDKVAEKLKEAFPDWAKAAYDKAAEKAKEAA

SEQ ID NO: 223 - protein, 34 aa, Artificial
  Note  Artificial = synthetic construct
KAFYDKVAEKLKEAFPDWAKAAYDKAAEKAKEAA

SEQ ID NO: 224 - protein, 33 aa, Artificial
  Note  Artificial = synthetic construct
AFYDKVAEKLKEAFPDWAKAAYDKAAEKAKEAA

SEQ ID NO: 225 - protein, 32 aa, Artificial
  Note  Artificial = synthetic construct
FYDKVAEKLKEAFPDWAKAAYDKAAEKAKEAA

SEQ ID NO: 226 - protein, 31 aa, Artificial
  Note  Artificial = synthetic construct
YDKVAEKLKEAFPDWAKAAYDKAAEKAKEAA

SEQ ID NO: 227 - protein, 36 aa, Artificial
  Note  Artificial = synthetic construct
DWLKAFYDKVAEKLKEAFPWAKAAYDKAAEKAKEAA

SEQ ID NO: 228 - protein, 35 aa, Artificial
  Note  Artificial = synthetic construct
DWLKAFYDKVAEKLKEAFPAKAAYDKAAEKAKEAA

SEQ ID NO: 229 - protein, 34 aa, Artificial
  Note  Artificial = synthetic construct
DWLKAFYDKVAEKLKEAFPKAAYDKAAEKAKEAA

SEQ ID NO: 230 - protein, 33 aa, Artificial
  Note  Artificial = synthetic construct
DWLKAFYDKVAEKLKEAFPAAYDKAAEKAKEAA

SEQ ID NO: 231 - protein, 32 aa, Artificial
  Note  Artificial = synthetic construct
DWLKAFYDKVAEKLKEAFPAYDKAAEKAKEAA

SEQ ID NO: 232 - protein, 31 aa, Artificial
  Note  Artificial = synthetic construct
DWLKAFYDKVAEKLKEAFPYDKAAEKAKEAA

SEQ ID NO: 233 - protein, 35 aa, Artificial
  Note  Artificial = synthetic construct
WLKAFYDKVAEKLKEAFPWAKAAYDKAAEKAKEAA

SEQ ID NO: 234 - protein, 33 aa, Artificial
  Note  Artificial = synthetic construct
LKAFYDKVAEKLKEAFPAKAAYDKAAEKAKEAA

SEQ ID NO: 235 - protein, 31 aa, Artificial
  Note  Artificial = synthetic construct
KAFYDKVAEKLKEAFPKAAYDKAAEKAKEAA

SEQ ID NO: 236 - protein, 29 aa, Artificial
  Note  Artificial = synthetic construct
AFYDKVAEKLKEAFPAAYDKAAEKAKEAA

SEQ ID NO: 237 - protein, 27 aa, Artificial
  Note  Artificial = synthetic construct
FYDKVAEKLKEAFPAYDKAAEKAKEAA

SEQ ID NO: 238 - protein, 25 aa, Artificial
  Note  Artificial = synthetic construct
YDKVAEKLKEAFPYDKAAEKAKEAA

SEQ ID NO: 239 - protein, 35 aa, Artificial
  Note  Artificial = synthetic construct
DWLKAFYDKVAEKLKEAPDWAKAAYDKAAEKAKEA

SEQ ID NO: 240 - protein, 33 aa, Artificial
  Note  Artificial = synthetic construct
DWLKAFYDKVAEKLKEPDWAKAAYDKAAEKAKE

SEQ ID NO: 241 - protein, 31 aa, Artificial
  Note  Artificial = synthetic construct
DWLKAFYDKVAEKLKPDWAKAAYDKAAEKAK

SEQ ID NO: 242 - protein, 29 aa, Artificial
  Note  Artificial = synthetic construct
DWLKAFYDKVAEKLPDWAKAAYDKAAEKA

SEQ ID NO: 243 - protein, 27 aa, Artificial
  Note  Artificial = synthetic construct
DWLKAFYDKVAEKPDWAKAAYDKAAEK

SEQ ID NO: 244 - protein, 25 aa, Artificial
  Note  Artificial = synthetic construct
DWLKAFYDKVAEPDWAKAAYDKAAE

SEQ ID NO: 245 - protein, 23 aa, Artificial
  Note  Artificial = synthetic construct
DWLKAFYDKVAPDWAKAAYDKAA

SEQ ID NO: 246 - protein, 21 aa, Artificial
  Note  Artificial = synthetic construct
DWLKAFYDKVPDWAKAAYDKA

SEQ ID NO: 247 - protein, 35 aa, Artificial
  Note  Artificial = synthetic construct
DWLKAFYDKVAEKLKEAPWAKAAYDKAAEKAKEAA

SEQ ID NO: 248 - protein, 33 aa, Artificial
  Note  Artificial = synthetic construct
DWLKAFYDKVAEKLKEPAKAAYDKAAEKAKEAA

SEQ ID NO: 249 - protein, 31 aa, Artificial
  Note  Artificial = synthetic construct
DWLKAFYDKVAEKLKPKAAYDKAAEKAKEAA

SEQ ID NO: 250 - protein, 29 aa, Artificial
  Note  Artificial = synthetic construct
DWLKAFYDKVAEKLPAAYDKAAEKAKEAA

SEQ ID NO: 251 - protein, 27 aa, Artificial
  Note  Artificial = synthetic construct
DWLKAFYDKVAEKPAYDKAAEKAKEAA

SEQ ID NO: 252 - protein, 25 aa, Artificial
  Note  Artificial = synthetic construct
DWLKAFYDKVAEPYDKAAEKAKEAA

SEQ ID NO: 253 - protein, 23 aa, Artificial
  Note  Artificial = synthetic construct
DWLKAFYDKVAPDKAAEKAKEAA

SEQ ID NO: 254 - protein, 35 aa, Artificial
  Note  Artificial = synthetic construct
WLKAFYDKVAEKLKEAFPDWAKAAYDKAAEKAKEA

SEQ ID NO: 255 - protein, 33 aa, Artificial
  Note  Artificial = synthetic construct
LKAFYDKVAEKLKEAFPDWAKAAYDKAAEKAKE

SEQ ID NO: 256 - protein, 33 aa, Artificial; identical to SEQ ID NO: 255
  Note  Artificial = synthetic construct

SEQ ID NO: 257 - protein, 29 aa, Artificial
  Note  Artificial = synthetic construct
AFYDKVAEKLKEAFPDWAKAAYDKAAEKA

SEQ ID NO: 258 - protein, 27 aa, Artificial
  Note  Artificial = synthetic construct
FYDKVAEKLKEAFPDWAKAAYDKAAEK

SEQ ID NO: 259 - protein, 25 aa, Artificial
  Note  Artificial = synthetic construct
YDKVAEKLKEAFPDWAKAAYDKAAE

SEQ ID NO: 260 - protein, 75 aa, Artificial
  Note  Artificial = synthetic construct
DWLKAFYDKVAEKLKEAFPDWAKAAYDKAAEKAKEAAPDWLKAFYDKVAEKLKEAFPDWAKAAYDKAAEKAKEAA

SEQ ID NO: 261 - protein, 113 aa, Artificial
  Note  Artificial = synthetic construct
DWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAA

SEQ ID NO: 262 - protein, 151 aa, Artificial
  Note  Artificial = synthetic construct
DWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAA

SEQ ID NO: 263 - protein, 56 aa, Artificial
  Note  Artificial = synthetic construct
DWLKAFYDKVAEKLKEAFPDWLKAFYDKVAEKLKEAFPDWAKAAYDKAAEKAKEAA

SEQ ID NO: 264 - protein, 56 aa, Artificial
  Note  Artificial = synthetic construct
DWLKAFYDKVAEKLKEAFPDWAKAAYDKAAEKAKEAAPDWAKAAYDKAAEKAKEAA

SEQ ID NO: 265 - protein, 37 aa, Artificial
  Note  Artificial = synthetic construct
DWAKAAYDKAAEKAKEAAPDWLKAFYDKVAEKLKEAF

SEQ ID NO: 266 - protein, 36 aa, Artificial
  Note  Artificial = synthetic construct
DWAKAAYDKAAEKAKEAAPDWLKAFYDKVAEKLKEA

SEQ ID NO: 267 - protein, 35 aa, Artificial
  Note  Artificial = synthetic construct
DWAKAAYDKAAEKAKEAAPDWLKAFYDKVAEKLKE

SEQ ID NO: 268 - protein, 34 aa, Artificial
  Note  Artificial = synthetic construct
DWAKAAYDKAAEKAKEAAPDWLKAFYDKVAEKLK

SEQ ID NO: 269 - protein, 33 aa, Artificial
  Note  Artificial = synthetic construct
DWAKAAYDKAAEKAKEAAPDWLKAFYDKVAEKL

SEQ ID NO: 270 - protein, 32 aa, Artificial
  Note  Artificial = synthetic construct
DWAKAAYDKAAEKAKEAAPDWLKAFYDKVAEK

SEQ ID NO: 271 - protein, 31 aa, Artificial
  Note  Artificial = synthetic construct
DWAKAAYDKAAEKAKEAAPDWLKAFYDKVAE

SEQ ID NO: 272 - protein, 36 aa, Artificial
  Note  Artificial = synthetic construct
DWLKAFYDKVAEKLKEAPDWLKAFYDKVAEKLKEAF

SEQ ID NO: 273 - protein, 35 aa, Artificial
  Note  Artificial = synthetic construct
DWLKAFYDKVAEKLKEPDWLKAFYDKVAEKLKEAF

SEQ ID NO: 274 - protein, 34 aa, Artificial
  Note  Artificial = synthetic construct
DWLKAFYDKVAEKLKPDWLKAFYDKVAEKLKEAF

SEQ ID NO: 275 - protein, 33 aa, Artificial
  Note  Artificial = synthetic construct
DWLKAFYDKVAEKLPDWLKAFYDKVAEKLKEAF

SEQ ID NO: 276 - protein, 32 aa, Artificial
  Note  Artificial = synthetic construct
DWLKAFYDKVAEKPDWLKAFYDKVAEKLKEAF

SEQ ID NO: 277 - protein, 31 aa, Artificial
  Note  Artificial = synthetic construct
DWLKAFYDKVAEPDWLKAFYDKVAEKLKEAF

SEQ ID NO: 278 - protein, 30 aa, Artificial
  Note  Artificial = synthetic construct
DWLKAFYDKVAPDWLKAFYDKVAEKLKEAF

SEQ ID NO: 279 - protein, 36 aa, Artificial
  Note  Artificial = synthetic construct
DWAKAAYDKAAEKAKEAAPWLKAFYDKVAEKLKEAF

SEQ ID NO: 280 - protein, 35 aa, Artificial
  Note  Artificial = synthetic construct
DWAKAAYDKAAEKAKEAAPLKAFYDKVAEKLKEAF

SEQ ID NO: 281 - protein, 34 aa, Artificial
  Note  Artificial = synthetic construct
DWAKAAYDKAAEKAKEAAPKAFYDKVAEKLKEAF

SEQ ID NO: 282 - protein, 33 aa, Artificial
  Note  Artificial = synthetic construct
DWAKAAYDKAAEKAKEAAPAFYDKVAEKLKEAF

SEQ ID NO: 283 - protein, 32 aa, Artificial
  Note  Artificial = synthetic construct
DWAKAAYDKAAEKAKEAAPFYDKVAEKLKEAF

SEQ ID NO: 284 - protein, 30 aa, Artificial
  Note  Artificial = synthetic construct
DWAKAAYDKAAEKAKEAAPDKVAEKLKEAF

SEQ ID NO: 285 - protein, 36 aa, Artificial
  Note  Artificial = synthetic construct
WAKAAYDKAAEKAKEAAPDWLKAFYDKVAEKLKEAF

SEQ ID NO: 286 - protein, 35 aa, Artificial
  Note  Artificial = synthetic construct
AKAAYDKAAEKAKEAAPDWLKAFYDKVAEKLKEAF

SEQ ID NO: 287 - protein, 34 aa, Artificial
  Note  Artificial = synthetic construct
KAAYDKAAEKAKEAAPDWLKAFYDKVAEKLKEAF

SEQ ID NO: 288 - protein, 33 aa, Artificial
  Note  Artificial = synthetic construct
AAYDKAAEKAKEAAPDWLKAFYDKVAEKLKEAF

SEQ ID NO: 289 - protein, 32 aa, Artificial
  Note  Artificial = synthetic construct
AYDKAAEKAKEAAPDWLKAFYDKVAEKLKEAF

SEQ ID NO: 290 - protein, 31 aa, Artificial
  Note  Artificial = synthetic construct
YDKAAEKAKEAAPDWLKAFYDKVAEKLKEAF

SEQ ID NO: 291 - protein, 35 aa, Artificial
  Note  Artificial = synthetic construct
WAKAAYDKAAEKAKEAAPWLKAFYDKVAEKLKEAF

SEQ ID NO: 292 - protein, 33 aa, Artificial
  Note  Artificial = synthetic construct
AKAAYDKAAEKAKEAAPLKAFYDKVAEKLKEAF

SEQ ID NO: 293 - protein, 31 aa, Artificial
  Note  Artificial = synthetic construct
KAAYDKAAEKAKEAAPKAFYDKVAEKLKEAF

SEQ ID NO: 294 - protein, 29 aa, Artificial
  Note  Artificial = synthetic construct
AAYDKAAEKAKEAAPAFYDKVAEKLKEAF

SEQ ID NO: 295 - protein, 27 aa, Artificial
  Note  Artificial = synthetic construct
AYDKAAEKAKEAAPFYDKVAEKLKEAF

SEQ ID NO: 296 - protein, 25 aa, Artificial
  Note  Artificial = synthetic construct
YDKAAEKAKEAAPYDKVAEKLKEAF

SEQ ID NO: 297 - protein, 35 aa, Artificial
  Note  Artificial = synthetic construct
WAKAAYDKAAEKAKEAAPDWLKAFYDKVAEKLKEA

SEQ ID NO: 298 - protein, 33 aa, Artificial
  Note  Artificial = synthetic construct
AKAAYDKAAEKAKEAAPDWLKAFYDKVAEKLKE

SEQ ID NO: 299 - protein, 31 aa, Artificial
  Note  Artificial = synthetic construct
KAAYDKAAEKAKEAAPDWLKAFYDKVAEKLK

SEQ ID NO: 300 - protein, 29 aa, Artificial
  Note  Artificial = synthetic construct
AAYDKAAEKAKEAAPDWLKAFYDKVAEKL

SEQ ID NO: 301 - protein, 27 aa, Artificial
  Note  Artificial = synthetic construct
AYDKAAEKAKEAAPDWLKAFYDKVAEK

SEQ ID NO: 302 - protein, 25 aa, Artificial
  Note  Artificial = synthetic construct
YDKAAEKAKEAAPDWLKAFYDKVAE

SEQ ID NO: 303 - protein, 35 aa, Artificial
  Note  Artificial = synthetic construct
DWAKAAYDKAAEKAKEAPWLKAFYDKVAEKLKEAF

SEQ ID NO: 304 - protein, 33 aa, Artificial
  Note  Artificial = synthetic construct
DWAKAAYDKAAEKAKEPLKAFYDKVAEKLKEAF

SEQ ID NO: 305 - protein, 31 aa, Artificial
  Note  Artificial = synthetic construct
DWAKAAYDKAAEKAKPKAFYDKVAEKLKEAF

SEQ ID NO: 306 - protein, 29 aa, Artificial
  Note  Artificial = synthetic construct
DWAKAAYDKAAEKAPAFYDKVAEKLKEAF

SEQ ID NO: 307 - protein, 27 aa, Artificial
  Note  Artificial = synthetic construct
DWAKAAYDKAAEKPFYDKVAEKLKEAF

SEQ ID NO: 308 - protein, 25 aa, Artificial
  Note  Artificial = synthetic construct
DWAKAAYDKAAEPYDKVAEKLKEAF

SEQ ID NO: 309 - protein, 75 aa, Artificial
  Note  Artificial = synthetic construct
DWAKAAYDKAAEKAKEAAPDWLKAFYDKVAEKLKEAFPDWAKAAYDKAAEKAKEAAPDWLKAFYDKVAEKLKEAF

SEQ ID NO: 310 - protein, 113 aa, Artificial
  Note  Artificial = synthetic construct
DWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAF

SEQ ID NO: 311 - protein, 151 aa, Artificial
  Note  Artificial = synthetic construct
DWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAFPDWAKAAYDKAAEKAKEAAPDWLKAFYDKVAEKLKEAF

SEQ ID NO: 312 - protein, 56 aa, Artificial
  Note  Artificial = synthetic construct
DWAKAAYDKAAEKAKEAAPDWAKAAYDKAAEKAKEAAPDWLKAFYDKVAEKLKEAF

SEQ ID NO: 313 - protein, 56 aa, Artificial
  Note  Artificial = synthetic construct
DWAKAAYDKAAEKAKEAAPDWLKAFYDKVAEKLKEAFPDWLKAFYDKVAEKLKEAF

SEQ ID NO: 314 - protein, 37 aa, Artificial
  Note  Artificial = synthetic construct
FAEKLKEAVKDYFAKLWDPDWAKAAYDKAAEKAKEAA

SEQ ID NO: 315 - protein, 37 aa, Artificial
  Note  Artificial = synthetic construct
DWLKAFYDKVAEKLKEAFPAAEKAKEAAKDYAAKAWD

SEQ ID NO: 316 - protein, 37 aa, Artificial
  Note  Artificial = synthetic construct
FAEKLKEAVKDYFAKLWDPAAEKAKEAAKDYAAKAWD

SEQ ID NO: 317 - protein, 37 aa, Artificial
  Note  Artificial = synthetic construct
AAEKAKEAAKDYAAKAWDPFAEKLKEAVKDYFAKLWD

SEQ ID NO: 318 - protein, 37 aa, Artificial
  Note  Artificial = synthetic construct
DWAKAAYDKAAEKAKEAAPFAEKLKEAVKDYFAKLWD

SEQ ID NO: 319 - protein, 37 aa, Artificial
  Note  Artificial = synthetic construct
DWLKAFYDKVAEKLKEAFPDWAKAAYDKAAEKAKEAX

SEQ ID NO: 320 - protein, 37 aa, Artificial
  Note  Artificial = synthetic construct
DWLKAFYDKVAEKLKEAFPDWAKAAYDKAAEKAKEXA

SEQ ID NO: 321 - protein, 37 aa, Artificial
  Note  Artificial = synthetic construct
DWLKAFYDKVAEKLKEAFPDWAKAAYDKAAEKAKXAA

SEQ ID NO: 322 - protein, 37 aa, Artificial
  Note  Artificial = synthetic construct
DWLKAFYDKVAEKLKEAFPDWAKAAYDKAAEKAXEAA

SEQ ID NO: 323 - protein, 37 aa, Artificial
  Note  Artificial = synthetic construct
DWLKAFYDKVAEKLKEAFPDWAKAAYDKAAEKXKEAA

SEQ ID NO: 324 - protein, 37 aa, Artificial
  Note  Artificial = synthetic construct
DWLKAFYDKVAEKLKEAFPDWAKAAYDKAAEXAKEAA

SEQ ID NO: 325 - protein, 37 aa, Artificial
  Note  Artificial = synthetic construct
DWLKAFYDKVAEKLKEAFPDWAKAAYDKAAEKXKEXX

SEQ ID NO: 326 - protein, 37 aa, Artificial
  Note  Artificial = synthetic construct
DWLKAFYDKVAEKLKEAFPDWAKAAYDKAAEKXKEXA

SEQ ID NO: 327 - protein, 37 aa, Artificial
  Note  Artificial = synthetic construct
DWLKAFYDKVAEKLKEAFPDWAKAAYDKAAEKXKEAX

SEQ ID NO: 328 - protein, 37 aa, Artificial
  Note  Artificial = synthetic construct
DWLKAFYDKVAEKLKEAFPDWAKAAYDKAAEXXKEAX

SEQ ID NO: 329 - protein, 37 aa, Artificial
  Note  Artificial = synthetic construct
DWLKAFYDKVAEKLKEAFPDWAKAAYDKAAEXAXEAX

SEQ ID NO: 330 - protein, 37 aa, Artificial
  Note  Artificial = synthetic construct
DWLKAFYDKVAEKLKEAFPDWAKAAYDKAAEKAKXAX

SEQ ID NO: 331 - protein, 37 aa, Artificial
  Note  Artificial = synthetic construct
DWLKAFYDKVAEKLKEAFPDWAKAAYDKAAEXAKXAA

SEQ ID NO: 332 - protein, 37 aa, Artificial
  Note  Artificial = synthetic construct
DWLKAFYDKVAEKLKEAFPDWAKAAYDKAAEXAXEXA

SEQ ID NO: 333 - protein, 37 aa, Artificial
  Note  Artificial = synthetic construct
DWLKAFYDKVAEKLKEAFPDWAKAAYDKAAEKXKXAX

SEQ ID NO: 334 - protein, 9 aa, Artificial
  Note  Artificial = synthetic construct
XRGGSVLVT

SEQ ID NO: 335 - protein, 9 aa, Artificial
  Note  Artificial = synthetic construct
PXGGSVLVT

SEQ ID NO: 336 - protein, 9 aa, Artificial
  Note  Artificial = synthetic construct
PRXGSVLVT

SEQ ID NO: 337 - protein, 9 aa, Artificial
  Note  Artificial = synthetic construct
PRGXSVLVT

SEQ ID NO: 338 - protein, 9 aa, Artificial
  Note  Artificial = synthetic construct
PRGGXVLVT

SEQ ID NO: 339 - protein, 9 aa, Artificial
  Note  Artificial = synthetic construct
PRGGSXLVT

SEQ ID NO: 340 - protein, 9 aa, Artificial
  Note  Artificial = synthetic construct
XVLVSGGRP

SEQ ID NO: 341 - protein, 9 aa, Artificial
  Note  Artificial = synthetic construct
TXLVSGGRP

SEQ ID NO: 342 - protein, 9 aa, Artificial
  Note  Artificial = synthetic construct
TVXVSGGRP

SEQ ID NO: 343 - protein, 9 aa, Artificial
  Note  Artificial = synthetic construct
TVLXSGGRP

SEQ ID NO: 344 - protein, 9 aa, Artificial
  Note  Artificial = synthetic construct
TVLVXGGRP

SEQ ID NO: 345 - protein, 9 aa, Artificial
  Note  Artificial = synthetic construct
TVLVSXGRP

SEQ ID NO: 346 - protein, 6 aa, Artificial
  Note  Artificial = synthetic construct
WWRWWW

SEQ ID NO: 347 - protein, 6 aa, Artificial
  Note  Artificial = synthetic construct
WWWRWW

SEQ ID NO: 348 - protein, 6 aa, Artificial
  Note  Artificial = synthetic construct
WWWWWR

SEQ ID NO: 349 - protein, 6 aa, Artificial
  Note  Artificial = synthetic construct
RWWWWW

SEQ ID NO: 350 - protein, 12 aa, Artificial
  Note  Artificial = synthetic construct
WRWWWWWRWWWW

SEQ ID NO: 351 - protein, 18 aa, Artificial
  Note  Artificial = synthetic construct
WRWWWWWRWWWWWRWWWW

SEQ ID NO: 352 - protein, 12 aa, Artificial
  Note  Artificial = synthetic construct
WWRWWWWWRWWW

SEQ ID NO: 353 - protein, 18 aa, Artificial
  Note  Artificial = synthetic construct
WWRWWWWWRWWWWWRWWW

SEQ ID NO: 354 - protein, 12 aa, Artificial
  Note  Artificial = synthetic construct
WWWRWWWWWRWW

SEQ ID NO: 355 - protein, 18 aa, Artificial
  Note  Artificial = synthetic construct
WWWRWWWWWRWWWWWRWW

SEQ ID NO: 356 - protein, 12 aa, Artificial
  Note  Artificial = synthetic construct
WWWWRWWWWWRW

SEQ ID NO: 357 - protein, 18 aa, Artificial
  Note  Artificial = synthetic construct
WWWWRWWWWWRWWWWWRW

SEQ ID NO: 358 - protein, 12 aa, Artificial
  Note  Artificial = synthetic construct
WWWWWRWWWWWR

SEQ ID NO: 359 - protein, 18 aa, Artificial
  Note  Artificial = synthetic construct
WWWWWRWWWWWRWWWWWR

SEQ ID NO: 360 - protein, 12 aa, Artificial
  Note  Artificial = synthetic construct
RWWWWWRWWWWW

SEQ ID NO: 361 - protein, 18 aa, Artificial
  Note  Artificial = synthetic construct
RWWWWWRWWWWWRWWWWW

SEQ ID NO: 362 - protein, 12 aa, Artificial
  Note  Artificial = synthetic construct
WRWWWWWWRWWW

SEQ ID NO: 363 - protein, 18 aa, Artificial
  Note  Artificial = synthetic construct
WRWWWWWWRWWWWWWRWW

SEQ ID NO: 364 - protein, 12 aa, Artificial
  Note  Artificial = synthetic construct
WRWWWWWWWWRW

SEQ ID NO: 365 - protein, 18 aa, Artificial
  Note  Artificial = synthetic construct
WRWWWWWWWWRWWWWWWR

SEQ ID NO: 366 - protein, 6 aa, Artificial
  Note  Artificial = synthetic construct
XRWWWW

SEQ ID NO: 367 - protein, 6 aa, Artificial
  Note  Artificial = synthetic construct
WXWWWW

SEQ ID NO: 368 - protein, 6 aa, Artificial
  Note  Artificial = synthetic construct
WRXWWW

SEQ ID NO: 369 - protein, 6 aa, Artificial
  Note  Artificial = synthetic construct
WRWXWW

SEQ ID NO: 370 - protein, 6 aa, Artificial
  Note  Artificial = synthetic construct
WRWWXW

SEQ ID NO: 371 - protein, 6 aa, Artificial
  Note  Artificial = synthetic construct
WRWWWX

SEQ ID NO: 372 - protein, 6 aa, Artificial
  Note  Artificial = synthetic construct
XWRWWW

SEQ ID NO: 373 - protein, 6 aa, Artificial
  Note  Artificial = synthetic construct
WXRWWW

SEQ ID NO: 374 - protein, 6 aa, Artificial
  Note  Artificial = synthetic construct
WWXWWW

SEQ ID NO: 375 - protein, 6 aa, Artificial
  Note  Artificial = synthetic construct
WWRXWW

SEQ ID NO: 376 - protein, 6 aa, Artificial
  Note  Artificial = synthetic construct
WWRWXW

SEQ ID NO: 377 - protein, 6 aa, Artificial
  Note  Artificial = synthetic construct
WWRWWX

SEQ ID NO: 378 - protein, 6 aa, Artificial
  Note  Artificial = synthetic construct
XWWRWW

SEQ ID NO: 379 - protein, 6 aa, Artificial
  Note  Artificial = synthetic construct
WXWRWW

SEQ ID NO: 380 - protein, 6 aa, Artificial
  Note  Artificial = synthetic construct
WWXRWW

SEQ ID NO: 381 - protein, 6 aa, Artificial
  Note  Artificial = synthetic construct
WWWXWW

SEQ ID NO: 382 - protein, 6 aa, Artificial
  Note  Artificial = synthetic construct
WWWRXW

SEQ ID NO: 383 - protein, 6 aa, Artificial
  Note  Artificial = synthetic construct
WWWRWX

SEQ ID NO: 384 - protein, 15 aa, Artificial
  Note  Artificial = synthetic construct
PRGGSVLVTWRWWWW

SEQ ID NO: 385 - protein, 15 aa, Artificial
  Note  Artificial = synthetic construct
XRGGSVLVTWRWWWW

SEQ ID NO: 386 - protein, 15 aa, Artificial
  Note  Artificial = synthetic construct
PXGGSVLVTWRWWWW

SEQ ID NO: 387 - protein, 15 aa, Artificial
  Note  Artificial = synthetic construct
PRXGSVLVTWRWWWW

SEQ ID NO: 388 - protein, 15 aa, Artificial
  Note  Artificial = synthetic construct
PRGXSVLVTWRWWWW

SEQ ID NO: 389 - protein, 15 aa, Artificial
  Note  Artificial = synthetic construct
PRGGXVLVTWRWWWW

SEQ ID NO: 390 - protein, 15 aa, Artificial
  Note  Artificial = synthetic construct
PRGGSXLVTWRWWWW

SEQ ID NO: 391 - protein, 15 aa, Artificial
  Note  Artificial = synthetic construct
WRWWWWPRGGSVLVT

SEQ ID NO: 392 - protein, 15 aa, Artificial
  Note  Artificial = synthetic construct
WRWWWWXRGGSVLVT

SEQ ID NO: 393 - protein, 15 aa, Artificial
  Note  Artificial = synthetic construct
WRWWWWPXGGSVLVT

SEQ ID NO: 394 - protein, 15 aa, Artificial
  Note  Artificial = synthetic construct
WRWWWWPRXGSVLVT

SEQ ID NO: 395 - protein, 15 aa, Artificial
  Note  Artificial = synthetic construct
WRWWWWPRGXSVLVT

SEQ ID NO: 396 - protein, 15 aa, Artificial
  Note  Artificial = synthetic construct
WRWWWWPRGGXVLVT

SEQ ID NO: 397 - protein, 14 aa, Artificial
  Note  Artificial = synthetic construct
RWWWWPRGGSXLVT

SEQ ID NO: 398 - protein, 15 aa, Artificial
  Note  Artificial = synthetic construct
PRGGSVLVTXRWWWW

SEQ ID NO: 399 - protein, 15 aa, Artificial
  Note  Artificial = synthetic construct
PRGGSVLVTWXWWWW

SEQ ID NO: 400 - protein, 15 aa, Artificial
  Note  Artificial = synthetic construct
PRGGSVLVTWRXWWW

SEQ ID NO: 401 - protein, 15 aa, Artificial
  Note  Artificial = synthetic construct
PRGGSVLVTWRWXWW

SEQ ID NO: 402 - protein, 15 aa, Artificial
  Note  Artificial = synthetic construct
PRGGSVLVTWRWWXW

SEQ ID NO: 403 - protein, 15 aa, Artificial
  Note  Artificial = synthetic construct
PRGGSVLVTWRWWWX

SEQ ID NO: 404 - protein, 45 aa, Artificial
  Note  Artificial = synthetic construct
WRWWWWXPDWLKAFYDKVAEKLKEAFPDWAKAAYDKAAEKAKEAA

SEQ ID NO: 405 - protein, 46 aa, Artificial
  Note  Artificial = synthetic construct
WRWWWWXXPDWLKAFYDKVAEKLKEAFPDWAKAAYDKAAEKAKEAA

SEQ ID NO: 406 - protein, 47 aa, Artificial
  Note  Artificial = synthetic construct
WRWWWWXXXPDWLKAFYDKVAEKLKEAFPDWAKAAYDKAAEKAKEAA

SEQ ID NO: 407 - protein, 48 aa, Artificial
  Note  Artificial = synthetic construct
WRWWWWXXXXPDWLKAFYDKVAEKLKEAFPDWAKAAYDKAAEKAKEAA

SEQ ID NO: 408 - protein, 44 aa, Artificial
  Note  Artificial = synthetic construct
WRWWWWPDWLKAFYDKVAEKLKEAFPDWAKAAYDKAAEKAKEAA

SEQ ID NO: 409 - protein, 43 aa, Artificial
  Note  Artificial = synthetic construct
WRWWWWDWLKAFYDKVAEKLKEAFPDWAKAAYDKAAEKAKEAA

SEQ ID NO: 410 - protein, 53 aa, Artificial
  Note  Artificial = synthetic construct
WRWWWWXPDWLKAFYDKVAEKLKEAFPDWAKAAYDKAAEKAKEAAPXWRWWWW

SEQ ID NO: 411 - protein, 55 aa, Artificial
  Note  Artificial = synthetic construct
WRWWWWXXPDWLKAFYDKVAEKLKEAFPDWAKAAYDKAAEKAKEAAPXXWRWWWW

SEQ ID NO: 412 - protein, 57 aa, Artificial
  Note  Artificial = synthetic construct
WRWWWWXXXPDWLKAFYDKVAEKLKEAFPDWAKAAYDKAAEKAKEAAPXXXWRWWWW

SEQ ID NO: 413 - protein, 59 aa, Artificial
  Note  Artificial = synthetic construct
WRWWWWXXXXPDWLKAFYDKVAEKLKEAFPDWAKAAYDKAAEKAKEAAPXXXXWRWWWW

SEQ ID NO: 414 - protein, 53 aa, Artificial
  Note  Artificial = synthetic construct
WRWWWWXPDWLKAFYDKVAEKLKEAFPDWAKAAYDKAAEKAKEAAPXWWWWRW

SEQ ID NO: 415 - protein, 55 aa, Artificial
  Note  Artificial = synthetic construct
WRWWWWXXPDWLKAFYDKVAEKLKEAFPDWAKAAYDKAAEKAKEAAPXXWWWWRW

SEQ ID NO: 416 - protein, 57 aa, Artificial
  Note  Artificial = synthetic construct
WRWWWWXXXPDWLKAFYDKVAEKLKEAFPDWAKAAYDKAAEKAKEAAPXXXWWWWRW

SEQ ID NO: 417 - protein, 59 aa, Artificial
  Note  Artificial = synthetic construct
WRWWWWXXXXPDWLKAFYDKVAEKLKEAFPDWAKAAYDKAAEKAKEAAPXXXXWWWWRW

SEQ ID NO: 418 - protein, 56 aa, Artificial
  Note  Artificial = synthetic construct
WRWWWWXPDWLKAFYDKVAEKLKEAFPDWAKAAYDKAAEKAKEAAXPPRGGSVLVT

SEQ ID NO: 419 - protein, 56 aa, Artificial
  Note  Artificial = synthetic construct
XRWWWWXPDWLKAFYDKVAEKLKEAFPDWAKAAYDKAAEKAKEAAXPPRGGSVLVX

SEQ ID NO: 420 - protein, 55 aa, Artificial
  Note  Artificial = synthetic construct
WRWWWWXPDWLKAFYDKVAEKLKEAFPDWAKAAYDKAAEKAKEAAXTVLVSGGRP

SEQ ID NO: 421 - protein, 55 aa, Artificial
  Note  Artificial = synthetic construct
XRWWWWXPDWLKAFYDKVAEKLKEAFPDWAKAAYDKAAEKAKEAAXTVLVSGGRX

SEQ ID NO: 422 - protein, 56 aa, Artificial
  Note  Artificial = synthetic construct
PRGGSVLVTXPWRWWWWXPDWLKAFYDKVAEKLKEAFPDWAKAAYDKAAEKAKEAA

SEQ ID NO: 423 - protein, 55 aa, Artificial
  Note  Artificial = synthetic construct
XXXXXXXXXXXXXXXXXXXXXXXXXXXXXXXXXXXXXXXXXXXXXXXXXXXXXXX

SEQ ID NO: 424 - protein, 56 aa, Artificial
  Note  Artificial = synthetic construct
XRGGSVLVTXPWRWWWWXPDWLKAFYDKVAEKLKEAFPDWAKAAYDKAAEKAKEAX

SEQ ID NO: 425 - protein, 57 aa, Artificial
  Note  Artificial = synthetic construct
PRGGSVLVTXPWRWWWWXXPDWLKAFYDKVAEKLKEAFPDWAKAAYDKAAEKAKEAA

SEQ ID NO: 426 - protein, 58 aa, Artificial
  Note  Artificial = synthetic construct
PRGGSVLVTXPWRWWWWXXXPDWLKAFYDKVAEKLKEAFPDWAKAAYDKAAEKAKEAA

SEQ ID NO: 427 - protein, 59 aa, Artificial
  Note  Artificial = synthetic construct
PRGGSVLVTXPWRWWWWXXXXPDWLKAFYDKVAEKLKEAFPDWAKAAYDKAAEKAKEAA

SEQ ID NO: 428 - protein, 63 aa, Artificial
  Note  Artificial = synthetic construct
PRGGSVLVTXWRWWWWXPDWLKAFYDKVAEKLKEAFPDWAKAAYDKAAEKAKEAAPXWRWWWW

SEQ ID NO: 429 - protein, 66 aa, Artificial
  Note  Artificial = synthetic construct
PRGGSVLVTXPWRWWWWXXPDWLKAFYDKVAEKLKEAFPDWAKAAYDKAAEKAKEAAPXXWRWWWW

SEQ ID NO: 430 - protein, 68 aa, Artificial
  Note  Artificial = synthetic construct
PRGGSVLVTXPWRWWWWXXXPDWLKAFYDKVAEKLKEAFPDWAKAAYDKAAEKAKEAAPXXXWRWWWW

SEQ ID NO: 431 - protein, 70 aa, Artificial
  Note  Artificial = synthetic construct
PRGGSVLVTXPWRWWWWXXXXPDWLKAFYDKVAEKLKEAFPDWAKAAYDKAAEKAKEAAPXXXXWRWWWW

SEQ ID NO: 432 - protein, 63 aa, Artificial
  Note  Artificial = synthetic construct
PRGGSVLVTXPWRWWWWXDWLKAFYDKVAEKLKEAFPDWAKAAYDKAAEKAKEAAPXWWWWRW

SEQ ID NO: 433 - protein, 66 aa, Artificial
  Note  Artificial = synthetic construct
PRGGSVLVTXPWRWWWWXXPDWLKAFYDKVAEKLKEAFPDWAKAAYDKAAEKAKEAAPXXWWWWRW

SEQ ID NO: 434 - protein, 68 aa, Artificial
  Note  Artificial = synthetic construct
PRGGSVLVTXPWRWWWWXXXPDWLKAFYDKVAEKLKEAFPDWAKAAYDKAAEKAKEAAPXXXWWWWRW

SEQ ID NO: 435 - protein, 70 aa, Artificial
  Note  Artificial = synthetic construct
PRGGSVLVTXPWRWWWWXXXXPDWLKAFYDKVAEKLKEAFPDWAKAAYDKAAEKAKEAAPXXXXWWWWRW

SEQ ID NO: 436 - protein, 55 aa, Artificial
  Note  Artificial = synthetic construct
PRGGSVLVTPWRWWWWXPDWLKAFYDKVAEKLKEAFPDWAKAAYDKAAEKAKEAA

SEQ ID NO: 437 - protein, 56 aa, Artificial
  Note  Artificial = synthetic construct
PRGGSVLVTPWRWWWWXXPDWLKAFYDKVAEKLKEAFPDWAKAAYDKAAEKAKEAA

SEQ ID NO: 438 - protein, 57 aa, Artificial
  Note  Artificial = synthetic construct
PRGGSVLVTPWRWWWWXXXPDWLKAFYDKVAEKLKEAFPDWAKAAYDKAAEKAKEAA

SEQ ID NO: 439 - protein, 58 aa, Artificial
  Note  Artificial = synthetic construct
PRGGSVLVTPWRWWWWXXXXPDWLKAFYDKVAEKLKEAFPDWAKAAYDKAAEKAKEAA

SEQ ID NO: 440 - protein, 63 aa, Artificial
  Note  Artificial = synthetic construct
PRGGSVLVTPWRWWWWXPDWLKAFYDKVAEKLKEAFPDWAKAAYDKAAEKAKEAAPXWRWWWW

SEQ ID NO: 441 - protein, 65 aa, Artificial
  Note  Artificial = synthetic construct
PRGGSVLVTPWRWWWWXXPDWLKAFYDKVAEKLKEAFPDWAKAAYDKAAEKAKEAAPXXWRWWWW

SEQ ID NO: 442 - protein, 67 aa, Artificial
  Note  Artificial = synthetic construct
PRGGSVLVTPWRWWWWXXXPDWLKAFYDKVAEKLKEAFPDWAKAAYDKAAEKAKEAAPXXXWRWWWW

SEQ ID NO: 443 - protein, 69 aa, Artificial
  Note  Artificial = synthetic construct
PRGGSVLVTPWRWWWWXXXXPDWLKAFYDKVAEKLKEAFPDWAKAAYDKAAEKAKEAAPXXXXWRWWWW

SEQ ID NO: 444 - protein, 48 aa, Artificial
  Note  Artificial = synthetic construct
PRGGSVLVTXPDWLKAFYDKVAEKLKEAFPDWAKAAYDKAAEKAKEAA

SEQ ID NO: 445 - protein, 49 aa, Artificial
  Note  Artificial = synthetic construct
PRGGSVLVTXXPDWLKAFYDKVAEKLKEAFPDWAKAAYDKAAEKAKEAA

SEQ ID NO: 446 - protein, 50 aa, Artificial
  Note  Artificial = synthetic construct
PRGGSVLVTXXXPDWLKAFYDKVAEKLKEAFPDWAKAAYDKAAEKAKEAA

SEQ ID NO: 447 - protein, 51 aa, Artificial
  Note  Artificial = synthetic construct
PRGGSVLVTXXXXPDWLKAFYDKVAEKLKEAFPDWAKAAYDKAAEKAKEAA

SEQ ID NO: 448 - protein, 47 aa, Artificial
  Note  Artificial = synthetic construct
PRGGSVLVTPDWLKAFYDKVAEKLKEAFPDWAKAAYDKAAEKAKEAA

SEQ ID NO: 449 - protein, 46 aa, Artificial
  Note  Artificial = synthetic construct
PRGGSVLVTDWLKAFYDKVAEKLKEAFPDWAKAAYDKAAEKAKEAA

SEQ ID NO: 450 - protein, 75 aa, Artificial
  Note  Artificial = synthetic construct
PRGGSVLVTXPWRWWWWXPDWLKAFYDKVAEKLKEAFPDWAKAAYDKAAEKAKEAAPXWRWWWWPXPRGGSVLVT

SEQ ID NO: 451 - protein, 77 aa, Artificial
  Note  Artificial = synthetic construct
PRGGSVLVTXPWRWWWWXXPDWLKAFYDKVAEKLKEAFPDWAKAAYDKAAEKAKEAAPXXWRWWWWPXPRGGSVLVT

SEQ ID NO: 452 - protein, 79 aa, Artificial
  Note  Artificial = synthetic construct
PRGGSVLVTXPWRWWWWXXXPDWLKAFYDKVAEKLKEAFPDWAKAAYDKAAEKAKEAAPXXXWRWWWWPXPRGGSVLVT

SEQ ID NO: 453 - protein, 81 aa, Artificial
  Note  Artificial = synthetic construct
PRGGSVLVTXPWRWWWWXXXXPDWLKAFYDKVAEKLKEAFPDWAKAAYDKAAEKAKEAAPXXXXWRWWWWPXPRGGSVLVT

SEQ ID NO: 454 - protein, 45 aa, Artificial
  Note  Artificial = synthetic construct
DWLKAFYDKVAEKLKEAFPDWAKAAYDKAAEKAKEAAPXWRWWWW

SEQ ID NO: 455 - protein, 45 aa, Artificial
  Note  Artificial = synthetic construct
WLKAFYDKVAEKLKEAFPDWAKAAYDKAAEKAKEAAPXXWRWWWW

SEQ ID NO: 456 - protein, 47 aa, Artificial
  Note  Artificial = synthetic construct
DWLKAFYDKVAEKLKEAFPDWAKAAYDKAAEKAKEAAPXXXWRWWWW

SEQ ID NO: 457 - protein, 45 aa, Artificial
  Note  Artificial = synthetic construct
DWLKAFYDKVAEKLKEAFPDWAKAAYDKAAEKAKEAAPXWWWWRW

SEQ ID NO: 458 - protein, 46 aa, Artificial
  Note  Artificial = synthetic construct
DWLKAFYDKVAEKLKEAFPDWAKAAYDKAAEKAKEAAPXXWWWWRW

SEQ ID NO: 459 - protein, 47 aa, Artificial
  Note  Artificial = synthetic construct
DWLKAFYDKVAEKLKEAFPDWAKAAYDKAAEKAKEAAPXXXWWWWRW

SEQ ID NO: 460 - protein, 48 aa, Artificial
  Note  Artificial = synthetic construct
DWLKAFYDKVAEKLKEAFPDWAKAAYDKAAEKAKEAAPXXXXWWWWRW

SEQ ID NO: 461 - protein, 43 aa, Artificial
  Note  Artificial = synthetic construct
DWLKAFYDKVAEKLKEAFPDWAKAAYDKAAEKAKEAAWWWWRW

SEQ ID NO: 462 - protein, 44 aa, Artificial
  Note  Artificial = synthetic construct
DWLKAFYDKVAEKLKEAFPDWAKAAYDKAAEKAKEAAPWWWWRW

SEQ ID NO: 463 - protein, 56 aa, Artificial
  Note  Artificial = synthetic construct
DWLKAFYDKVAEKLKEAFPDWAKAAYDKAAEKAKEAAPXWRWWWWPXPRGGSVLVT

SEQ ID NO: 464 - protein, 57 aa, Artificial
  Note  Artificial = synthetic construct
DWLKAFYDKVAEKLKEAFPDWAKAAYDKAAEKAKEAAPXXWRWWWWPXPRGGSVLVT

SEQ ID NO: 465 - protein, 58 aa, Artificial
  Note  Artificial = synthetic construct
DWLKAFYDKVAEKLKEAFPDWAKAAYDKAAEKAKEAAPXXXWRWWWWPXPRGGSVLVT

SEQ ID NO: 466 - protein, 59 aa, Artificial
  Note  Artificial = synthetic construct
DWLKAFYDKVAEKLKEAFPDWAKAAYDKAAEKAKEAAPXXXXWRWWWWPXPRGGSVLVT

SEQ ID NO: 467 - protein, 55 aa, Artificial
  Note  Artificial = synthetic construct
DWLKAFYDKVAEKLKEAFPDWAKAAYDKAAEKAKEAAPXWRWWWWPPRGGSVLVT

SEQ ID NO: 468 - protein, 56 aa, Artificial
  Note  Artificial = synthetic construct
DWLKAFYDKVAEKLKEAFPDWAKAAYDKAAEKAKEAAPXXWRWWWWPPRGGSVLVT

SEQ ID NO: 469 - protein, 57 aa, Artificial
  Note  Artificial = synthetic construct
DWLKAFYDKVAEKLKEAFPDWAKAAYDKAAEKAKEAAPXXXWRWWWWPPRGGSVLVT

SEQ ID NO: 470 - protein, 58 aa, Artificial
  Note  Artificial = synthetic construct
DWLKAFYDKVAEKLKEAFPDWAKAAYDKAAEKAKEAAPXXXXWRWWWWPPRGGSVLVT

SEQ ID NO: 471 - protein, 56 aa, Artificial
  Note  Artificial = synthetic construct
DWLKAFYDKVAEKLKEAFPDWAKAAYDKAAEKAKEAAPXWRWWWWPXTVLVSGGRP

SEQ ID NO: 472 - protein, 57 aa, Artificial
  Note  Artificial = synthetic construct
DWLKAFYDKVAEKLKEAFPDWAKAAYDKAAEKAKEAAPXXWRWWWWPXTVLVSGGRP

SEQ ID NO: 473 - protein, 58 aa, Artificial
  Note  Artificial = synthetic construct
DWLKAFYDKVAEKLKEAFPDWAKAAYDKAAEKAKEAAPXXXWRWWWWPXTVLVSGGRP

SEQ ID NO: 474 - protein, 59 aa, Artificial
  Note  Artificial = synthetic construct
DWLKAFYDKVAEKLKEAFPDWAKAAYDKAAEKAKEAAPXXXXWRWWWWPXTVLVSGGRP

SEQ ID NO: 475 - protein, 54 aa, Artificial
  Note  Artificial = synthetic construct
WLKAFYDKVAEKLKEAFPDWAKAAYDKAAEKAKEAAPXWRWWWWPTVLVSGGRP

SEQ ID NO: 476 - protein, 56 aa, Artificial
  Note  Artificial = synthetic construct
DWLKAFYDKVAEKLKEAFPDWAKAAYDKAAEKAKEAAPXXWRWWWWPTVLVSGGRP

SEQ ID NO: 477 - protein, 57 aa, Artificial
  Note  Artificial = synthetic construct
DWLKAFYDKVAEKLKEAFPDWAKAAYDKAAEKAKEAAPXXXWRWWWWPTVLVSGGRP

SEQ ID NO: 478 - protein, 58 aa, Artificial
  Note  Artificial = synthetic construct
DWLKAFYDKVAEKLKEAFPDWAKAAYDKAAEKAKEAAPXXXXWRWWWWPTVLVSGGRP

SEQ ID NO: 479 - protein, 48 aa, Artificial
  Note  Artificial = synthetic construct
DWLKAFYDKVAEKLKEAFPDWAKAAYDKAAEKAKEAAPXPRGGSVLVT

SEQ ID NO: 480 - protein, 49 aa, Artificial
  Note  Artificial = synthetic construct
DWLKAFYDKVAEKLKEAFPDWAKAAYDKAAEKAKEAAPXXPRGGSVLVT

SEQ ID NO: 481 - protein, 50 aa, Artificial
  Note  Artificial = synthetic construct
DWLKAFYDKVAEKLKEAFPDWAKAAYDKAAEKAKEAAPXXXPRGGSVLVT

SEQ ID NO: 482 - protein, 51 aa, Artificial
  Note  Artificial = synthetic construct
DWLKAFYDKVAEKLKEAFPDWAKAAYDKAAEKAKEAAPXXXXPRGGSVLVT

SEQ ID NO: 483 - protein, 47 aa, Artificial
  Note  Artificial = synthetic construct
DWLKAFYDKVAEKLKEAFPDWAKAAYDKAAEKAKEAAXPRGGSVLVT

SEQ ID NO: 484 - protein, 48 aa, Artificial
  Note  Artificial = synthetic construct
DWLKAFYDKVAEKLKEAFPDWAKAAYDKAAEKAKEAAXXPRGGSVLVT

SEQ ID NO: 485 - protein, 49 aa, Artificial
  Note  Artificial = synthetic construct
DWLKAFYDKVAEKLKEAFPDWAKAAYDKAAEKAKEAAXXXPRGGSVLVT

SEQ ID NO: 486 - protein, 50 aa, Artificial
  Note  Artificial = synthetic construct
DWLKAFYDKVAEKLKEAFPDWAKAAYDKAAEKAKEAAXXXXPRGGSVLVT

SEQ ID NO: 487 - protein, 46 aa, Artificial
  Note  Artificial = synthetic construct
DWLKAFYDKVAEKLKEAFPDWAKAAYDKAAEKAKEAAPRGGSVLVT

SEQ ID NO: 488 - protein, 47 aa, Artificial
  Note  Artificial = synthetic construct
DWLKAFYDKVAEKLKEAFPDWAKAAYDKAAEKAKEAAPPRGGSVLVT

SEQ ID NO: 489 - protein, 48 aa, Artificial
  Note  Artificial = synthetic construct
DWLKAFYDKVAEKLKEAFPDWAKAAYDKAAEKAKEAAPXTVLVSGGRP

SEQ ID NO: 490 - protein, 49 aa, Artificial
  Note  Artificial = synthetic construct
DWLKAFYDKVAEKLKEAFPDWAKAAYDKAAEKAKEAAPXXTVLVSGGRP

SEQ ID NO: 491 - protein, 50 aa, Artificial
  Note  Artificial = synthetic construct
DWLKAFYDKVAEKLKEAFPDWAKAAYDKAAEKAKEAAPXXXTVLVSGGRP

SEQ ID NO: 492 - protein, 51 aa, Artificial
  Note  Artificial = synthetic construct
DWLKAFYDKVAEKLKEAFPDWAKAAYDKAAEKAKEAAPXXXXTVLVSGGRP

SEQ ID NO: 493 - protein, 47 aa, Artificial
  Note  Artificial = synthetic construct
DWLKAFYDKVAEKLKEAFPDWAKAAYDKAAEKAKEAAXTVLVSGGRP

SEQ ID NO: 494 - protein, 48 aa, Artificial
  Note  Artificial = synthetic construct
DWLKAFYDKVAEKLKEAFPDWAKAAYDKAAEKAKEAAXXTVLVSGGRP

SEQ ID NO: 495 - protein, 49 aa, Artificial
  Note  Artificial = synthetic construct
DWLKAFYDKVAEKLKEAFPDWAKAAYDKAAEKAKEAAXXXTVLVSGGRP

SEQ ID NO: 496 - protein, 50 aa, Artificial
  Note  Artificial = synthetic construct
DWLKAFYDKVAEKLKEAFPDWAKAAYDKAAEKAKEAAXXXXTVLVSGGRP

SEQ ID NO: 497 - protein, 46 aa, Artificial
  Note  Artificial = synthetic construct
DWLKAFYDKVAEKLKEAFPDWAKAAYDKAAEKAKEAATVLVSGGRP

SEQ ID NO: 498 - protein, 47 aa, Artificial
  Note  Artificial = synthetic construct
DWLKAFYDKVAEKLKEAFPDWAKAAYDKAAEKAKEAAPTVLVSGGRP

SEQ ID NO: 499 - protein, 56 aa, Artificial
  Note  Artificial = synthetic construct
XRGGSVLVTXPWRWWWWXPDWLKAFYDKVAEKLKEAFPDWAKAAYDKAAEKAKEAA

SEQ ID NO: 500 - protein, 56 aa, Artificial
  Note  Artificial = synthetic construct
PXGGSVLVTXPWRWWWWXPDWLKAFYDKVAEKLKEAFPDWAKAAYDKAAEKAKEAA

SEQ ID NO: 501 - protein, 56 aa, Artificial
  Note  Artificial = synthetic construct
PRXGSVLVTXPWRWWWWXPDWLKAFYDKVAEKLKEAFPDWAKAAYDKAAEKAKEAA

SEQ ID NO: 502 - protein, 56 aa, Artificial
  Note  Artificial = synthetic construct
PRGXSVLVTXPWRWWWWXPDWLKAFYDKVAEKLKEAFPDWAKAAYDKAAEKAKEAA

SEQ ID NO: 503 - protein, 56 aa, Artificial
  Note  Artificial = synthetic construct
PRGGXVLVTXPWRWWWWXPDWLKAFYDKVAEKLKEAFPDWAKAAYDKAAEKAKEAA

SEQ ID NO: 504 - protein, 56 aa, Artificial
  Note  Artificial = synthetic construct
PRGGSXLVTXPWRWWWWXPDWLKAFYDKVAEKLKEAFPDWAKAAYDKAAEKAKEAA

SEQ ID NO: 505 - protein, 63 aa, Artificial
  Note  Artificial = synthetic construct
XRGGSVLVTPWRWWWWXPDWLKAFYDKVAEKLKEAFPDWAKAAYDKAAEKAKEAAPXWWWWRW

SEQ ID NO: 506 - protein, 65 aa, Artificial
  Note  Artificial = synthetic construct
PXGGSVLVTPWRWWWWXXPDWLKAFYDKVAEKLKEAFPDWAKAAYDKAAEKAKEAAPXXWWWWRW

SEQ ID NO: 507 - protein, 67 aa, Artificial
  Note  Artificial = synthetic construct
PRXGSVLVTPWRWWWWXXXPDWLKAFYDKVAEKLKEAFPDWAKAAYDKAAEKAKEAAPXXXWWWWRW

SEQ ID NO: 508 - protein, 69 aa, Artificial
  Note  Artificial = synthetic construct
PRGXSVLVTPWRWWWWXXXXPDWLKAFYDKVAEKLKEAFPDWAKAAYDKAAEKAKEAAPXXXXWWWWRW

SEQ ID NO: 509 - protein, 55 aa, Artificial
  Note  Artificial = synthetic construct
PRGGXVLVTPWRWWWWXPDWLKAFYDKVAEKLKEAFPDWAKAAYDKAAEKAKEAA

SEQ ID NO: 510 - protein, 55 aa, Artificial
  Note  Artificial = synthetic construct
PRGGSXLVTPWRWWWWXPDWLKAFYDKVAEKLKEAFPDWAKAAYDKAAEKAKEAA

SEQ ID NO: 511 - protein, 17 aa, Artificial
  Note  Artificial = synthetic construct
XRGGSVLVTXPWRWWWW

SEQ ID NO: 512 - protein, 17 aa, Artificial
  Note  Artificial = synthetic construct
PXGGSVLVTXPWRWWWW

SEQ ID NO: 513 - protein, 17 aa, Artificial
  Note  Artificial = synthetic construct
PRXGSVLVTXPWRWWWW

SEQ ID NO: 514 - protein, 17 aa, Artificial
  Note  Artificial = synthetic construct
PRGXSVLVTXPWRWWWW

SEQ ID NO: 515 - protein, 17 aa, Artificial
  Note  Artificial = synthetic construct
PRGGXVLVTXPWRWWWW

SEQ ID NO: 516 - protein, 17 aa, Artificial
  Note  Artificial = synthetic construct
PRGGSXLVTXPWRWWWW

SEQ ID NO: 517 - protein, 16 aa, Artificial
  Note  Artificial = synthetic construct
PRGGSXLVTPWRWWWW

SEQ ID NO: 518 - protein, 15 aa, Artificial
  Note  Artificial = synthetic construct
XRGGSVLVTWRWWWW

SEQ ID NO: 519 - protein, 15 aa, Artificial
  Note  Artificial = synthetic construct
PXGGSVLVTWRWWWW

SEQ ID NO: 520 - protein, 15 aa, Artificial
  Note  Artificial = synthetic construct
PRXGSVLVTWRWWWW

SEQ ID NO: 521 - protein, 15 aa, Artificial
  Note  Artificial = synthetic construct
PRGXSVLVTWRWWWW

SEQ ID NO: 522 - protein, 15 aa, Artificial
  Note  Artificial = synthetic construct
PRGGXVLVTWRWWWW

SEQ ID NO: 523 - protein, 15 aa, Artificial
  Note  Artificial = synthetic construct
PRGGSXLVTWRWWWW

SEQ ID NO: 524 - protein, 45 aa, Artificial
  Note  Artificial = synthetic construct
WRWWWWXPDWLKAFYDKVAEKLKEAFPDWAKAAYDKAAEKAKEAX

SEQ ID NO: 525 - protein, 45 aa, Artificial
  Note  Artificial = synthetic construct
WRWWWWXPDWLKAFYDKVAEKLKEAFPDWAKAAYDKAAEKAKEXA

SEQ ID NO: 526 - protein, 44 aa, Artificial
  Note  Artificial = synthetic construct
WRWWWWXDWLKAFYDKVAEKLKEAFPDWAKAAYDKAAEKAKXAA

SEQ ID NO: 527 - protein, 45 aa, Artificial
  Note  Artificial = synthetic construct
WRWWWWXPDWLKAFYDKVAEKLKEAFPDWAKAAYDKAAEKAXEAA

SEQ ID NO: 528 - protein, 45 aa, Artificial
  Note  Artificial = synthetic construct
WRWWWWXPDWLKAFYDKVAEKLKEAFPDWAKAAYDKAAEKXKEAA

SEQ ID NO: 529 - protein, 44 aa, Artificial
  Note  Artificial = synthetic construct
WRWWWWXDWLKAFYDKVAEKLKEAFPDWAKAAYDKAAEXAKEAA

SEQ ID NO: 530 - protein, 44 aa, Artificial
  Note  Artificial = synthetic construct
WRWWWWPDWLKAFYDKVAEKLKEAFPDWAKAAYDKAAEKAKEAX

SEQ ID NO: 531 - protein, 44 aa, Artificial
  Note  Artificial = synthetic construct
WRWWWWPDWLKAFYDKVAEKLKEAFPDWAKAAYDKAAEKAKEXA

SEQ ID NO: 532 - protein, 44 aa, Artificial
  Note  Artificial = synthetic construct
WRWWWWPDWLKAFYDKVAEKLKEAFPDWAKAAYDKAAEKAKXAA

SEQ ID NO: 533 - protein, 44 aa, Artificial
  Note  Artificial = synthetic construct
WRWWWWPDWLKAFYDKVAEKLKEAFPDWAKAAYDKAAEKAXEAA

SEQ ID NO: 534 - protein, 44 aa, Artificial
  Note  Artificial = synthetic construct
WRWWWWPDWLKAFYDKVAEKLKEAFPDWAKAAYDKAAEKXKEAA

SEQ ID NO: 535 - protein, 44 aa, Artificial
  Note  Artificial = synthetic construct
WRWWWWPDWLKAFYDKVAEKLKEAFPDWAKAAYDKAAEXAKEAA

SEQ ID NO: 536 - protein, 43 aa, Artificial
  Note  Artificial = synthetic construct
WRWWWWDWLKAFYDKVAEKLKEAFPDWAKAAYDKAAEKAKEAX

SEQ ID NO: 537 - protein, 43 aa, Artificial
  Note  Artificial = synthetic construct
WRWWWWDWLKAFYDKVAEKLKEAFPDWAKAAYDKAAEKAKEXA

SEQ ID NO: 538 - protein, 43 aa, Artificial
  Note  Artificial = synthetic construct
WRWWWWDWLKAFYDKVAEKLKEAFPDWAKAAYDKAAEKAKXAA

SEQ ID NO: 539 - protein, 43 aa, Artificial
  Note  Artificial = synthetic construct
WRWWWWDWLKAFYDKVAEKLKEAFPDWAKAAYDKAAEKAXEAA

SEQ ID NO: 540 - protein, 43 aa, Artificial
  Note  Artificial = synthetic construct
WRWWWWDWLKAFYDKVAEKLKEAFPDWAKAAYDKAAEKXKEAA

SEQ ID NO: 541 - protein, 43 aa, Artificial
  Note  Artificial = synthetic construct
WRWWWWDWLKAFYDKVAEKLKEAFPDWAKAAYDKAAEXAKEAA

SEQ ID NO: 542 - protein, 45 aa, Artificial
  Note  Artificial = synthetic construct
DWLKAFYDKVAEKLKEAFPDWAKAAYDKAAEKAKEAXPXWRWWWW

SEQ ID NO: 543 - protein, 44 aa, Artificial
  Note  Artificial = synthetic construct
WLKAFYDKVAEKLKEAFPDWAKAAYDKAAEKAKEXAPXWRWWWW

SEQ ID NO: 544 - protein, 45 aa, Artificial
  Note  Artificial = synthetic construct
DWLKAFYDKVAEKLKEAFPDWAKAAYDKAAEKAKXAAPXWRWWWW

SEQ ID NO: 545 - protein, 45 aa, Artificial
  Note  Artificial = synthetic construct
DWLKAFYDKVAEKLKEAFPDWAKAAYDKAAEKAXEAAPXWRWWWW

SEQ ID NO: 546 - protein, 44 aa, Artificial
  Note  Artificial = synthetic construct
WLKAFYDKVAEKLKEAFPDWAKAAYDKAAEKXKEAAPXWRWWWW

SEQ ID NO: 547 - protein, 45 aa, Artificial
  Note  Artificial = synthetic construct
DWLKAFYDKVAEKLKEAFPDWAKAAYDKAAEXAKEAAPXWRWWWW

SEQ ID NO: 548 - protein, 44 aa, Artificial
  Note  Artificial = synthetic construct
DWLKAFYDKVAEKLKEAFPDWAKAAYDKAAEKAKEAXPWRWWWW

SEQ ID NO: 549 - protein, 44 aa, Artificial
  Note  Artificial = synthetic construct
DWLKAFYDKVAEKLKEAFPDWAKAAYDKAAEKAKEXAPWRWWWW

SEQ ID NO: 550 - protein, 44 aa, Artificial
  Note  Artificial = synthetic construct
DWLKAFYDKVAEKLKEAFPDWAKAAYDKAAEKAKXAAPWRWWWW

SEQ ID NO: 551 - protein, 44 aa, Artificial
  Note  Artificial = synthetic construct
DWLKAFYDKVAEKLKEAFPDWAKAAYDKAAEKAXEAAPWRWWWW

SEQ ID NO: 552 - protein, 44 aa, Artificial
  Note  Artificial = synthetic construct
DWLKAFYDKVAEKLKEAFPDWAKAAYDKAAEKXKEAAPWRWWWW

SEQ ID NO: 553 - protein, 44 aa, Artificial
  Note  Artificial = synthetic construct
DWLKAFYDKVAEKLKEAFPDWAKAAYDKAAEXAKEAAPWRWWWW

SEQ ID NO: 554 - protein, 43 aa, Artificial
  Note  Artificial = synthetic construct
DWLKAFYDKVAEKLKEAFPDWAKAAYDKAAEKAKEAXWRWWWW

SEQ ID NO: 555 - protein, 43 aa, Artificial
  Note  Artificial = synthetic construct
DWLKAFYDKVAEKLKEAFPDWAKAAYDKAAEKAKEXAWRWWWW

SEQ ID NO: 556 - protein, 43 aa, Artificial
  Note  Artificial = synthetic construct
DWLKAFYDKVAEKLKEAFPDWAKAAYDKAAEKAKXAAWRWWWW

SEQ ID NO: 557 - protein, 43 aa, Artificial
  Note  Artificial = synthetic construct
DWLKAFYDKVAEKLKEAFPDWAKAAYDKAAEKAXEAAWRWWWW

SEQ ID NO: 558 - protein, 43 aa, Artificial
  Note  Artificial = synthetic construct
DWLKAFYDKVAEKLKEAFPDWAKAAYDKAAEKXKEAAWRWWWW

SEQ ID NO: 559 - protein, 43 aa, Artificial
  Note  Artificial = synthetic construct
DWLKAFYDKVAEKLKEAFPDWAKAAYDKAAEXAKEAAWRWWWW

SEQ ID NO: 560 - protein, 56 aa, Artificial
  Note  Artificial = synthetic construct
PRGGSVLVTXPWRWWWWXPDWLKAFYDKVAEKLKEAFPDWAKAAYDKAAEKAKEAX

SEQ ID NO: 561 - protein, 56 aa, Artificial
  Note  Artificial = synthetic construct
PRGGSVLVTXPWRWWWWXPDWLKAFYDKVAEKLKEAFPDWAKAAYDKAAEKAKEXA

SEQ ID NO: 562 - protein, 56 aa, Artificial
  Note  Artificial = synthetic construct
PRGGSVLVTXPWRWWWWXPDWLKAFYDKVAEKLKEAFPDWAKAAYDKAAEKAKXAA

SEQ ID NO: 563 - protein, 56 aa, Artificial
  Note  Artificial = synthetic construct
PRGGSVLVTXPWRWWWWXPDWLKAFYDKVAEKLKEAFPDWAKAAYDKAAEKAXEAA

SEQ ID NO: 564 - protein, 56 aa, Artificial
  Note  Artificial = synthetic construct
PRGGSVLVTXPWRWWWWXPDWLKAFYDKVAEKLKEAFPDWAKAAYDKAAEKXKEAA

SEQ ID NO: 565 - protein, 56 aa, Artificial
  Note  Artificial = synthetic construct
PRGGSVLVTXPWRWWWWXPDWLKAFYDKVAEKLKEAFPDWAKAAYDKAAEXAKEAA

SEQ ID NO: 566 - protein, 56 aa, Artificial
  Note  Artificial = synthetic construct
PXGGSVLVTXPWRWWWWXPDWLKAFYDKVAEKLKEAFPDWAKAAYDKAAEKAKEXA

SEQ ID NO: 567 - protein, 56 aa, Artificial
  Note  Artificial = synthetic construct
PRXGSVLVTXPWRWWWWXPDWLKAFYDKVAEKLKEAFPDWAKAAYDKAAEKAKXAA

SEQ ID NO: 568 - protein, 56 aa, Artificial
  Note  Artificial = synthetic construct
PRGXSVLVTXPWRWWWWXPDWLKAFYDKVAEKLKEAFPDWAKAAYDKAAEKAXEAA

SEQ ID NO: 569 - protein, 56 aa, Artificial
  Note  Artificial = synthetic construct
PRGGXVLVTXPWRWWWWXPDWLKAFYDKVAEKLKEAFPDWAKAAYDKAAEKXKEAA

SEQ ID NO: 570 - protein, 56 aa, Artificial
  Note  Artificial = synthetic construct
PRGGSXLVTXPWRWWWWXPDWLKAFYDKVAEKLKEAFPDWAKAAYDKAAEXAKEAA

SEQ ID NO: 571 - protein, 55 aa, Artificial
  Note  Artificial = synthetic construct
XRGGSVLVTPWRWWWWXPDWLKAFYDKVAEKLKEAFPDWAKAAYDKAAEKAKEAX

SEQ ID NO: 572 - protein, 55 aa, Artificial
  Note  Artificial = synthetic construct
PXGGSVLVTPWRWWWWXPDWLKAFYDKVAEKLKEAFPDWAKAAYDKAAEKAKEXA

SEQ ID NO: 573 - protein, 55 aa, Artificial
  Note  Artificial = synthetic construct
PRXGSVLVTPWRWWWWXPDWLKAFYDKVAEKLKEAFPDWAKAAYDKAAEKAKXAA

SEQ ID NO: 574 - protein, 55 aa, Artificial
  Note  Artificial = synthetic construct
PRGXSVLVTPWRWWWWXPDWLKAFYDKVAEKLKEAFPDWAKAAYDKAAEKAXEAA

SEQ ID NO: 575 - protein, 55 aa, Artificial
  Note  Artificial = synthetic construct
PRGGXVLVTPWRWWWWXPDWLKAFYDKVAEKLKEAFPDWAKAAYDKAAEKXKEAA

SEQ ID NO: 576 - protein, 55 aa, Artificial
  Note  Artificial = synthetic construct
PRGGSXLVTPWRWWWWXPDWLKAFYDKVAEKLKEAFPDWAKAAYDKAAEXAKEAA

SEQ ID NO: 577 - protein, 54 aa, Artificial
  Note  Artificial = synthetic construct
PRGGSVLVTXDWAKAAYDKAAEKAKEAAPDWLKAFYDKVAEKLKEAFPWRWWWW

SEQ ID NO: 578 - protein, 55 aa, Artificial
  Note  Artificial = synthetic construct
PRGGSVLVTXXDWAKAAYDKAAEKAKEAAPDWLKAFYDKVAEKLKEAFPWRWWWW

SEQ ID NO: 579 - protein, 56 aa, Artificial
  Note  Artificial = synthetic construct
PRGGSVLVTXXXDWAKAAYDKAAEKAKEAAPDWLKAFYDKVAEKLKEAFPWRWWWW

SEQ ID NO: 580 - protein, 57 aa, Artificial
  Note  Artificial = synthetic construct
PRGGSVLVTXXXXDWAKAAYDKAAEKAKEAAPDWLKAFYDKVAEKLKEAFPWRWWWW

SEQ ID NO: 581 - protein, 52 aa, Artificial
  Note  Artificial = synthetic construct
PRGGSVLVTDWAKAAYDKAAEKAKEAAPDWLKAFYDKVAEKLKEAFWRWWWW

SEQ ID NO: 582 - protein, 54 aa, Artificial
  Note  Artificial = synthetic construct
PRGGSVLVTPDWAKAAYDKAAEKAKEAAPDWLKAFYDKVAEKLKEAFPWRWWWW

SEQ ID NO: 583 - protein, 54 aa, Artificial
  Note  Artificial = synthetic construct
PRGGSVLVTXDWAKAAYDKAAEKAKEAAPDWLKAFYDKVAEKLKEAFXWRWWWW

SEQ ID NO: 584 - protein, 56 aa, Artificial
  Note  Artificial = synthetic construct
PRGGSVLVTXPWRWWWWXPDWAKAAYDKAAEKAKEAAPDWLKAFYDKVAEKLKEAF

SEQ ID NO: 585 - protein, 56 aa, Artificial
  Note  Artificial = synthetic construct
XRGGSVLVTXPWRWWWWXPDWAKAAYDKAAEKAKEAAPDWLKAFYDKVAEKLKEAX

SEQ ID NO: 586 - protein, 56 aa, Artificial
  Note  Artificial = synthetic construct
PRGGSVLVTXPDWAKAAYDKAAEKAKEAAPDWLKAFYDKVAEKLKEAFXPWRWWWW

SEQ ID NO: 587 - protein, 56 aa, Artificial
  Note  Artificial = synthetic construct
XRGGSVLVTXPDWAKAAYDKAAEKAKEAAPDWLKAFYDKVAEKLKEAFXPWRWWWX

SEQ ID NO: 588 - protein, 56 aa, Artificial
  Note  Artificial = synthetic construct
PRGGSVLVTXXDWAKAAYDKAAEKAKEAAPDWLKAFYDKVAEKLKEAFXXWRWWWW

SEQ ID NO: 589 - protein, 58 aa, Artificial
  Note  Artificial = synthetic construct
PRGGSVLVTXXXDWAKAAYDKAAEKAKEAAPDWLKAFYDKVAEKLKEAFXXXWRWWWW

SEQ ID NO: 590 - protein, 60 aa, Artificial
  Note  Artificial = synthetic construct
PRGGSVLVTXXXXDWAKAAYDKAAEKAKEAAPDWLKAFYDKVAEKLKEAFXXXXWRWWWW

SEQ ID NO: 591 - protein, 52 aa, Artificial; identical to SEQ ID NO: 581
  Note  Artificial = synthetic construct

SEQ ID NO: 592 - protein, 54 aa, Artificial
  Note  Artificial = synthetic construct
PRGGSVLVTXDWAKAAYDKAAEKAKEAAPDWLKAFYDKVAEKLKEAFPWWWWRW

SEQ ID NO: 593 - protein, 55 aa, Artificial
  Note  Artificial = synthetic construct
PRGGSVLVTXXDWAKAAYDKAAEKAKEAAPDWLKAFYDKVAEKLKEAFPWWWWRW

SEQ ID NO: 594 - protein, 56 aa, Artificial
  Note  Artificial = synthetic construct
PRGGSVLVTXXXDWAKAAYDKAAEKAKEAAPDWLKAFYDKVAEKLKEAFPWWWWRW

SEQ ID NO: 595 - protein, 57 aa, Artificial
  Note  Artificial = synthetic construct
PRGGSVLVTXXXXDWAKAAYDKAAEKAKEAAPDWLKAFYDKVAEKLKEAFPWWWWRW

SEQ ID NO: 596 - protein, 52 aa, Artificial
  Note  Artificial = synthetic construct
PRGGSVLVTDWAKAAYDKAAEKAKEAAPDWLKAFYDKVAEKLKEAFWWWWRW

SEQ ID NO: 597 - protein, 54 aa, Artificial
  Note  Artificial = synthetic construct
PRGGSVLVTPDWAKAAYDKAAEKAKEAAPDWLKAFYDKVAEKLKEAFPWWWWRW

SEQ ID NO: 598 - protein, 54 aa, Artificial
  Note  Artificial = synthetic construct
PRGGSVLVTXDWAKAAYDKAAEKAKEAAPDWLKAFYDKVAEKLKEAFXWWWWRW

SEQ ID NO: 599 - protein, 56 aa, Artificial
  Note  Artificial = synthetic construct
PRGGSVLVTXPDWAKAAYDKAAEKAKEAAPDWLKAFYDKVAEKLKEAFXPWWWWRW

SEQ ID NO: 600 - protein, 56 aa, Artificial
  Note  Artificial = synthetic construct
XRGGSVLVTPXDWAKAAYDKAAEKAKEAAPDWLKAFYDKVAEKLKEAFXPWWWWRX

SEQ ID NO: 601 - protein, 56 aa, Artificial
  Note  Artificial = synthetic construct
PRGGSVLVTXXDWAKAAYDKAAEKAKEAAPDWLKAFYDKVAEKLKEAFXXWWWWRW

SEQ ID NO: 602 - protein, 58 aa, Artificial
  Note  Artificial = synthetic construct
PRGGSVLVTXXXDWAKAAYDKAAEKAKEAAPDWLKAFYDKVAEKLKEAFXXXWWWWRW

SEQ ID NO: 603 - protein, 60 aa, Artificial
  Note  Artificial = synthetic construct
PRGGSVLVTXXXXDWAKAAYDKAAEKAKEAAPDWLKAFYDKVAEKLKEAFXXXXWWWWRW

SEQ ID NO: 604 - protein, 52 aa, Artificial; identical to SEQ ID NO: 596
  Note  Artificial = synthetic construct

SEQ ID NO: 605 - protein, 52 aa, Artificial
  Note  Artificial = synthetic construct
PRGGSVLVTDWAKAAYDKAAEKAKEAAPFAEKLKEAVKDYFAKLWDWRWWWW

SEQ ID NO: 606 - protein, 54 aa, Artificial
  Note  Artificial = synthetic construct
PRGGSVLVTPDWAKAAYDKAAEKAKEAAPFAEKLKEAVKDYFAKLWDPWRWWWW

SEQ ID NO: 607 - protein, 56 aa, Artificial
  Note  Artificial = synthetic construct
PRGGSVLVTXPDWAKAAYDKAAEKAKEAAPFAEKLKEAVKDYFAKLWDXPWRWWWW

SEQ ID NO: 608 - protein, 52 aa, Artificial
  Note  Artificial = synthetic construct
XRGGSVLVTDWAKAAYDKAAEKAKEAAPFAEKLKEAVKDYFAKLWDWRWWWX

SEQ ID NO: 609 - protein, 54 aa, Artificial
  Note  Artificial = synthetic construct
XRGGSVLVTPDWAKAAYDKAAEKAKEAAPFAEKLKEAVKDYFAKLWDPWRWWWX

SEQ ID NO: 610 - protein, 56 aa, Artificial
  Note  Artificial = synthetic construct
XRGGSVLVTXPDWAKAAYDKAAEKAKEAAPFAEKLKEAVKDYFAKLWDXPWRWWWX

SEQ ID NO: 611 - protein, 52 aa, Artificial
  Note  Artificial = synthetic construct
PRGGSVLVTDWAKAAYDKAAEKAKEAAPFAEKLKEAVKDYFAKLWDWWWWRW

SEQ ID NO: 612 - protein, 54 aa, Artificial
  Note  Artificial = synthetic construct
PRGGSVLVTPDWAKAAYDKAAEKAKEAAPFAEKLKEAVKDYFAKLWDPWWWWRW

SEQ ID NO: 613 - protein, 56 aa, Artificial
  Note  Artificial = synthetic construct
PRGGSVLVTXPDWAKAAYDKAAEKAKEAAPFAEKLKEAVKDYFAKLWDXPWWWWRW

SEQ ID NO: 614 - protein, 52 aa, Artificial
  Note  Artificial = synthetic construct
XRGGSVLVTDWAKAAYDKAAEKAKEAAPFAEKLKEAVKDYFAKLWDWWWWRX

SEQ ID NO: 615 - protein, 54 aa, Artificial
  Note  Artificial = synthetic construct
XRGGSVLVTPDWAKAAYDKAAEKAKEAAPFAEKLKEAVKDYFAKLWDPWWWWRX

SEQ ID NO: 616 - protein, 56 aa, Artificial
  Note  Artificial = synthetic construct
XRGGSVLVTXPDWAKAAYDKAAEKAKEAAPFAEKLKEAVKDYFAKLWDXPWWWWRX

SEQ ID NO: 617 - protein, 48 aa, Artificial; identical to SEQ ID NO: 118
  Note  Artificial = synthetic construct

SEQ ID NO: 618 - protein, 48 aa, Artificial; identical to SEQ ID NO: 121
  Note  Artificial = synthetic construct

SEQ ID NO: 619 - protein, 37 aa, Artificial; identical to SEQ ID NO: 40
  Note  Artificial = synthetic construct

SEQ ID NO: 620 - protein, 66 aa, Artificial; identical to SEQ ID NO: 57
  Note  Artificial = synthetic construct

SEQ ID NO: 621 - protein, 59 aa, Artificial; identical to SEQ ID NO: 154
  Note  Artificial = synthetic construct

SEQ ID NO: 622 - protein, 66 aa, Artificial; identical to SEQ ID NO: 69
  Note  Artificial = synthetic construct

SEQ ID NO: 623 - protein, 55 aa, Artificial; identical to SEQ ID NO: 155
  Note  Artificial = synthetic construct

SEQ ID NO: 624 - protein, 48 aa, Artificial; identical to SEQ ID NO: 130
  Note  Artificial = synthetic construct

SEQ ID NO: 625 - protein, 59 aa, Artificial; identical to SEQ ID NO: 162
  Note  Artificial = synthetic construct

SEQ ID NO: 626 - protein, 55 aa, Artificial; identical to SEQ ID NO: 163
  Note  Artificial = synthetic construct

SEQ ID NO: 627 - protein, 41 aa, Artificial; identical to SEQ ID NO: 41
  Note  Artificial = synthetic construct

SEQ ID NO: 628 - protein, 37 aa, Artificial; identical to SEQ ID NO: 42
  Note  Artificial = synthetic construct

SEQ ID NO: 629 - protein, 6 aa, Artificial
  Note  Artificial = synthetic construct
WKYMVX

(19) United States
(12) Patent Application Publication
(10) Pub. No.: US 2008/0199398 A1
(43) Pub. Date: Aug. 21, 2008

(54) Novel Peptides That Promote Lipid Efflux

(76) Inventor: H. Bryan Brewer, Potomac, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/764,619

(22) Filed: Jun. 18, 2007

Related U.S. Application Data
(60) Provisional application No. 60/814,466, filed on Jun. 16, 2006.
(60) Provisional application No. 60/847,586, filed on Sep. 26, 2006.
(60) Provisional application No. 60/858,073, filed on Nov. 10, 2006.

(51) Int. Cl. A61K 49/00; C07K 14/00; A61K 38/16; A61P 7/00

(52) U.S. Cl. 424/9.1; 530/324; 514/12

(57) ABSTRACT

Disclosed herein are peptides with domains that promote lipid efflux from cells and optionally possess at least one anti-inflammatory domain or a domain that stimulates LCAT activity. Provided herein are methods of using the peptides to treat or inhibit diseases including dyslipidemic disorders, stroke and myocardial infarction. Also provided are methods of detecting plaque in vessels using the labeled peptides of the present invention.

### PRIOR RELATED APPLICATIONS

This applcation claims the priority of U.S. Provisional Patent Application Nos. 60/847,856 filed Sept. 26, 2006 and 60/858,073 filed Nov. 10, 2006, ehich are each incorporated by reference herein their entirety.

### FIELD OF THE INVENTION

This present invention relates to peptides or peptide analogs that contain functional domains and promote lipid efflux. These peptides or peptide analogs optionally contain one or more anti-inflammatory domian and one or more domain that affects lecithin cholesterol acyltransferase (LCAT) activity. The dislcosuer further relates to methods for administering these peptides in the treatment and prevention of dyslipdemic and vacular disorders. The disclosurefurther relates to methods for using these peptides in assays and in methods of imaging sites of association of these ppeptides with receptors and with sites of lipid deposition.

### BACKGROUND OF THE INVENTION

Clearance of excess chlosteral from cells by high density lipoproteins (HDK) is facilitated by the interaction of HDL apolipoprotein with cell-surface binding sites or receptors. Research has demostrated an inverse correlation between the occurance of atherosclerosis events and levels of HDL and its most abundant protein constituent, apoliprotein A-I (apoA-I) (Panagotopulos et al., Biol. Chem . 277:3947739484, 2002). ApoA-I has beed shown to promote lipid efflux from ABCA1-transfected cells (Wang et al., J. Biol. Chem. 275:33053-33058, 2000; Hamon et al., Cell Biol. 2:399-406, 2000; and Realey et al., Bio chem. Biophys. Red. Commun. 280:818-823, 2001). However, the nature of the interaction between apoA-I and ABCA1 is not fully understood.

There exsists a need for non-cytotoxic, synthetic peptide mimetics of apolipoproteins that promote specific lipid efflux from cells, perhaps by ABCA1-dependent pathway, for use in the treatment and prevention of cardiovascular diseases, such as atherosclerosis.

Inflammation is believed to contribute to a variety of disease processes, including vascular disease. Inflamation is believed tp contribute to the process of atherosclerosis,

and physicians often prescribe anti-inflammatory medicine, such as aspirin, to patients with atherosclerosis, in conjunction with statins, in an attempt to decrease the ongoing inflammatory process that contributes to atherosclerosis and vascular disease. What is needed are compounds that decrease inflammation.

LCAT is the major enzyme involved in the esterification of free cholesterol present in circulating plasma lipoproteins, and a major determinant of plasma HDL concentrations. What is needed are compounds that increase LCAT activity.

What is needed are new compositions that promote lipid efflux. What is also needed are new compositions with functional domains that promote lipid efflux and have anti-inflammatory properties and/or activity to modulate LCAT activity, or a combination of domains that have anti-inflammatory properties and the activity to modulate LCAT activity.

### SUMMARY OF THE INVENTION

The present invention solves these problems by providing novel peptide compositions with functional domains. In several embodiments, these novel peptide compositions promote lipid efflux and have anti-inflammatory properties. In several embodiments, these novel peptide compositions promote lipid efflux and have one or more anti-inflammatory domains. In several embodiments, these novel peptide compositions promote lipid efflux and have one or more domains that affect LCAT activity. In several embodiments, these novel peptide compositions promote lipid efflux and have one or more anti-inflammatory domains and one or more domains that affect LCAT activity.

These novel peptide compositions may be labeled and used in a variety of applications including the visualization of plaque in vessels. These novel peptide compositions also display low toxicity.

The peptides of the present invention may be combined with pharmaceutically acceptable carriers and administered to a human or an animal as a composition. Administration may be through any means described herein and includes but is not limited to parenteral and oral administration and also administration on a coated device such as a stent or catheter.

Also described herein is a method of treating dyslipidemic and vascular disorders in an animal or a human, including administering to the animal or the human a therapeutically effective amount of the peptides or peptide analogs thereof presented herein. Dyslipidemic and vascular disorders amenable to treatment with the peptides disclosed herein include, but are not limited to, hyperlipidemia, hyperlipoproteinemia, hypercholesterolemia, hypertriglyceridemia, HDL deficiency, apoA-I deficiency, coronary artery disease, atherosclerosis, myocardial infarction, stroke and inflammation secondary to stroke, ischemia, ischemic stroke, thrombotic stroke, peripheral vascular disease including peripheral arterial disease, restenosis, thrombosis, acute coronary syndrome, and reperfusion myocardial injury.

The peptides of the present invention may be labeled with labels known to one of ordinary skill in the art and used for numerous applications, including but not limited to use in imaging applications to visualize atherosclerotic plaque. Labels include but are not limited to colorimetric labels, radiodense labels and radioisotopic labels. Other uses include but are not limited to use in assays, such as ELISAs, Western blots, radioimmunoassays and radioreceptor assays.

The peptides of the present invention may be used to generate antisera using techniques known to one of ordinary skill in the art.

The amino acid sequences disclosed herein are shown using standard three letter codes for amino acids, as defined in 37 C.F.R. 1.822 and as commonly known to one of ordinary skill in the art. When the three letter designation for an amino acid is shown in three upper case letters, for example SER for serine, the SER is a D amino acid.

Several of the generic formulae described below refer to helical regions 5, 6, and 8 of ApoA-I. Helices 5, 6 and 8 of ApoA-I are as follows wherein each helix number is followed by the amino acid residues associated with that helix: 5:145-162; 6:167-184; 8:222-239. FIG. 1 shows the numbered amino acid sequence of ApoA-I.

In one embodiment, the peptides of the present invention are described by the following generic formula I:

(A-B-C)ₙ I

wherein A comprises helix 5 of ApoA-I, helix 6 of ApoA-I, or a modified form of helix 8 of ApoA-I, C comprises helix 8 of ApoA-I, B is a linking group that forms a loop between A and C and n is an integer from 1 to 10.

In one embodiment, A is helix 5 of ApoA-I and is SEQ ID NO: 1 Gly Glu Glu Met Arg Asp Arg Ala Arg Ala His Val Asp Ala Leu Arg Thr His, or a conservative substitution thereof. These amino acids may also appear in reverse orientation as in SEQ ID NO: 2 His Thr Arg Leu Ala Asp Val His Ala Arg Ala Arg Asp Arg Met Glu Glu Gly.

In another embodiment, A is helix 6 of ApoA-I and is SEQ ID NO: 3 Ser Asp Glu Leu Arg Gln Arg Leu Ala Ala Arg Leu Glu Ala Leu Lys Glu Asn, or a conservative substitution thereof. These amino acids may also appear in reverse orientation as in SEQ ID NO: 4 Asn Glu Lys Leu Ala Glu Leu Arg Ala Ala Leu Arg Gln Arg Leu Glu Asp Ser.

In one embodiment, A is a modified form of helix 8 of ApoA-I, also called 8′ herein, and is SEQ ID NO: 5 Leu Glu Ser Ala Lys Val Ser Ala Leu Ser Ala Leu Glu Glu Ala Thr Lys Lys, or a conservative substitution thereof. These amino acids may also appear in reverse orientation such that Lys is at the N-terminus and Leu is at the C-terminus as in SEQ ID NO: 6 Lys Lys Thr Ala Glu Glu Leu Ala Ser Leu Ala Ser Val Lys Ala Ser Glu Leu. This modification of helix 8 involves substitutions at positions 4 (Phe to Ala), 8 (Phe to Ala) and 15 (Tyr to Ala). It is to be understood that the present invention encompasses other amino acid substitutions at these locations. At positions 4 and 8, Phe may be substituted with Val, Leu, Gly, Thr, Ser or gamma aminobutyric acid (GABA: GABA is also designated as 4Abu herein). At position 15, Tyr may be substituted with Val, Leu, Gly, Thr, Ser or GABA. While not wanting to be bound by the following statement, it is believed that A, the modified form of helix 8 of ApoA-I, has a lower lipid affinity than C, the unmodified form of helix 8 of ApoA-I.

In one embodiment, B is Pro, SEQ ID NO: 7 Lys Leu Ser Pro Leu, SEQ ID NO: 8 Leu Ser Pro Leu, or SEQ ID NO: 9 Ser Pro Leu, or a conservative substitution thereof. These amino acids may also appear in reverse orientation for example as in SEQ ID NO: Leu Pro Ser Leu Lys, SEQ ID NO:11 Leu Pro Ser Leu, and SEQ ID NO: 12 Leu Pro Ser.

In one embodiment, C is helix 8 of ApoA-I and is SEQ ID NO: 13 Leu Glu Ser Phe Lys Val Ser Phe Leu Ser Ala Leu Glu Glu Tyr Thr Lys Lys, or a conservative substitution thereof. These amino acids may also appear in reverse orientation such that Lys is at the N-terminus and Leu is at the C-terminus as SEQ ID NO: 14 Lys Lys Thr Tyr Glu Glu Leu Ala Ser Leu Phe Ser Val Lys Phe Ser Glu Leu.

It is to be understood that A and C may be switched in location as in C-B-A.

In a further embodiment, peptides of the present invention are described by the following subgeneric formula II, in which one or more additional elements indicated as variables D, E, F and W, are added to formula I to make subgeneric formula II.

D-E-(A-B-C)ₙ-F-W II

(A-B-C)ₙ are as described in formula I above.

D is absent or present and is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

E is absent or present and is a group linking D and A and is Pro, SEQ ID NO: 10 Leu Pro Ser Leu Lys, or a conservative substitution thereof, provided that E is present only when D is present. These amino acids may also appear in reverse orientation as in SEQ ID NO: 7 Lys Leu Ser Pro Leu. When D is absent, E is absent.

F is absent or present and is a group linking C and W and is Pro, SEQ ID NO: 19 Ala Leu Ser Pro Leu, or a conservative substitution thereof, provided that F is present only when W is present. These amino acids may also appear in reverse orientation as in SEQ ID NO: 20 Leu Pro Ser Leu Ala. When W is absent, F is absent.

W is absent or present and is a peptide as defined in the present specification. In one embodiment, W is a peptide selected from the group consisting of SEQ ID NO: 21 Trp Arg Trp Trp Trp Trp), or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

As stated above, in generic formula II, variables D or W may be absent or present. In one embodiment, D is present and W is absent. In another embodiment, W is present and D is absent. In another embodiment, both D and W are present.

In yet another embodiment, W and D as described in formula III may be switched in location.

W-E-(A-B-C)ₙ-F-D III

In a further embodiment, peptides of the present invention are described by the following subgeneric formula IV, in which one or more additional elements indicated as variables G and H, are added to formula I to make subgeneric formula IV.

G-(A-B-C)ₙ-H IV

(A-B-C)ₙ are as described in formula I above,

G is absent or present and is a peptide as defined in the present specification. In one embodiment, G is SEQ ID NO: 9 Ser Pro Leu or a conservative substitution thereof. These amino acids may also appear in reverse orientation as in SEQ ID NO: 12 Leu Pro Ser. It is to be understood that one or more of the amino acids in the G peptide may be D amino acids.

H is absent or present and is a peptide as defined in the present specification. In one embodiment, H is SEQ ID NO: 23 Leu Asn Thr Gln or a conservative substitution thereof. These amino acids may also appear in reverse orientation as in SEQ ID NO: 24 Gln Thr Asn Leu. It is to be understood that one or more of the amino acids in the H peptide may be D amino acids.

In a further embodiment, peptides of the present invention are described by the following subgeneric formula V, in which one or more additional elements indicated as variables D, E, F, W, G and H are added to formula I to make subgeneric formula V.

D-E-G-(A-B-C)ₙ-H-F-W V

(A-B-C)ₙ are as described in formula I above,

D is absent or present and is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

E is absent or present and is a group linking D and A and is Pro, SEQ ID NO: 10 Leu Pro Ser Leu Lys, or a conservative substitution thereof, provided that E is present only when D is present. These amino acids may also appear in reverse orientation as in SEQ ID NO: 7 Lys Leu Ser Pro Leu. When D is absent, E is absent.

F is absent or present and is a group linking C and W and is Pro, SEQ ID NO: 19 Ala Leu Ser Pro Leu, or a conservative substitution thereof, provided that F is present only when W is present. These amino acids may also appear in reverse orientation as in SEQ ID NO: 20 Leu Pro Ser Leu Ala. When W is absent, F is absent.

W is absent or present and is a peptide as defined in the present specification. In one embodiment, W is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

G is absent or present and is a peptide as defined in the present specification provided that G is present when D is absent. In one embodiment, G is SEQ ID NO: 9 Ser Pro Leu or a conservative substitution thereof. These amino acids may also appear in reverse orientation as in SEQ ID NO: 12 Leu Pro Ser. It is to be understood that one or more of the amino acids in the G peptide may be D amino acids.

H is absent or present and is a peptide as defined in the present specification provided that H is present when W is absent. In one embodiment, H is SEQ ID NO: 23 Leu Asn Thr Gln or a conservative substitution thereof. These amino acids may also appear in reverse orientation as in SEQ ID NO: 24 Gln Thr Asn Leu. It is to be understood that one or more of the amino acids in the H peptide may be D amino acids.

As stated above, in generic formula V, variables D or W may be absent or present. In one embodiment, D is present and W is absent. In another embodiment, W is present and D is absent. In another embodiment, both D and W are present.

In a further embodiment, peptides of the present invention are described by formula VI,

D-I-W VI

D is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

I is a group linking D and W and is GABA, Pro, SEQ ID NO: 7 Lys Leu Ser Pro Leu, SEQ ID NO: 25 Leu Lys Leu Ser Pro Leu, or a conservative substitution thereof, or multiples thereof or combinations thereof. These amino acids may also appear in reverse orientation, for example, SEQ ID NO: 26 Leu Pro Ser Leu Lys Leu.

W is absent or present and is a peptide as defined in the present specification. In one embodiment, W is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

D and W may also be switched in location in D-I-W to form W-I-D.

In another embodiment, the peptides of the present invention are described by the following generic formula VII:

(D-R-S-W-T-N-O-(Xₙ-Y_{z}-Zₘ)ₛ-O′-N′-T′-W′-S′-R′-D′)ᵣ VII

wherein: n is an integer from 1 to 3, m is an integer from 1 to 3, r is an integer from 1 to 10, z is an integer from 1 to 13, and s is an integer from 1 to 5. It is to be understood that the letters in the generic formulae VII-XVIII or in components thereof are defined by the text that follows each letter and do not designate an individual amino acid.

D or D′ is individually absent or present and is a peptide as defined in the present specification. In one embodiment, D or D′ is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D or D′, namely SEQ ID NO: 17 Pro Arg Gly Gly Ser Val or SEQ ID NO: 18 Thr Val Leu Val Ser Gly may occur as D-amino acids;

R or R′ is individually absent or present and is a linking group comprised of at least one gamma aminobutyric acid (GABA), or one or more neutral amino acids. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for R or R′ include SEQ ID NO: 27 (Gly-Pro-Gly-Gly), and SEQ ID NO: 28 (Gly₄-Ser)_{y}, wherein x is an integer from about 1 to about 9 and y is an integer from about 1 to about 8. Suitable linking groups for R or R′ may be selected from the following without limitation:

GABA; GABA-GABA; GABA-GABA-GABA;

SEQ ID NO: 29

Gly Pro Gly Gly;

SEQ ID NO: 30

Gly Pro Gly Gly Gly Pro Gly Gly;

SEQ ID NO: 31

Gly Pro Gly Gly Gly Pro Gly Gly Gly Pro Gly Gly;

SEQ ID NO: 32

Gly Pro Gly Gly Gly Pro Gly Gly Gly Pro Gly Gly
Gly Pro Gly Gly;

SEQ ID NO: 33

Gly Gly Gly Gly Ser;

SEQ ID NO: 34

Gly Gly Gly Gly Ser Gly Gly Gly Gly Ser
or

SEQ ID NO: 35

Gly Gly Gly Gly Ser Gly Gly Gly Gly Ser Gly Gly
Gly Gly Ser;

S or S′ is individually absent or present and is a linking group comprised of amino acid residues of from 1 to 10 residues in length, wherein the amino acid residues are proline, alanine, leucine, lysine, serine, glycine, polymers thereof or combinations (co-polymers) thereof, or is comprised of an alkyl group (CH₂)ₙ, wherein n is an integer from 1-20;

W or W′ is individually absent or present and is a peptide as defined in the present specification. In one embodiment, W or W′ is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W or W′ peptide may be D amino acids;

T or T′ is individually absent or present and is a linking group comprised of at least one gamma aminobutyric acid (GABA), or one or more neutral amino acids. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for T or T′ include SEQ ID NO: 27 (Gly-Pro-Gly-Gly)ₓ and SEQ ID NO: 28 (Gly₄-Ser)_{y}, wherein x is an integer from about 1 to about 9 and y is an integer from about 1 to about 8. Suitable linking groups for T or T′ may be selected from the following without limitation: GABA; GABA-GABA; GABA-GABA-GABA; SEQ ID NO: 29 Gly Pro Gly Gly; SEQ ID NO: 30 Gly Pro Gly Gly Gly Pro Gly Gly; SEQ ID NO:31 Gly Pro Gly Gly Gly Pro Gly Gly Gly Pro Gly Gly; SEQ ID NO: 32 Gly Pro Gly Gly Gly Pro Gly Gly Gly Pro Gly Gly Gly Pro Gly Gly; SEQ ID NO: 33 Gly Gly Gly Gly Ser; SEQ ID NO: 34 Gly Gly Gly Gly Ser Gly Gly Gly Gly Ser or SEQ ID NO: 35 Gly Gly Gly Gly Ser Gly Gly Gly Gly Ser Gly Gly Gly Gly Ser;

N or N′ is individually absent or present and is a linking group comprised of amino acid residues of from 1 to 10 residues in length, wherein the amino acid residues are proline, alanine, leucine, serine, glycine, polymers thereof or combinations (co-polymers) thereof, or is comprised of an alkyl group (CH₂)ₙ, wherein n is an integer from 1-20;

O or O′ is individually absent or present and is a linking group comprised of at least one GABA, or one or more neutral amino acids, or combinations thereof. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for O or O′ include SEQ ID NO: 27 (Gly-Pro-Gly-Gly)ₓ and SEQ ID NO: 28 (Gly₄-Ser)_{y}, wherein x is an integer from about 1 to about 9 and y is an integer from about 1 to about 8. Suitable linking groups for O or O′ may be selected from the following without limitation: GABA; GABA-GABA; GABA-GABA-GABA; SEQ ID NO: 29 Gly Pro Gly Gly; SEQ ID NO: 30 Gly Pro Gly Gly Gly Pro Gly Gly; SEQ ID NO: 31 Gly Pro Gly Gly Gly Pro Gly Gly Gly Pro Gly Gly; SEQ ID NO: 32 Gly Pro Gly Gly Gly Pro Gly Gly Gly Pro Gly Gly Gly Pro Gly Gly; SEQ ID NO: 33 Gly Gly Gly Gly Ser; SEQ ID NO: 34 Gly Gly Gly Gly Ser Gly Gly Gly Gly Ser or SEQ ID NO: 35 Gly Gly Gly Gly Ser Gly Gly Gly Gly Ser Gly Gly Gly Gly Ser;

X is a peptide comprised of from 5 to 25 amino acid residues, provided an amphipathic alpha helix is obtained. Examples of X are provided below in the specification;

Y is absent or present and is a linking group comprised of amino acid(s) of from 1 to 10 residues in length, wherein the amino acid residues are proline, alanine, leucine, serine, glycine, lysine, polymers thereof or combinations (co-polymers) thereof, or is comprised of an alkyl group (CH₂)ₙ, wherein n is an integer from 1-20; z is an integer from 1 to 13 and refers to the number of times Y may be present in Xₙ-Y_{z}-Zₘ when n or m in Xₙ-Y_{z}-Zₘ are more than 1 or when s is more than 1;

Z is a peptide comprised of from 5 to 25 amino acids residues, provided an amphipathic alpha helix is obtained. Examples of Z are provided below in the specification, provided that more than one 1 amphipathic alpha helical domain is present when X and Z are taken in combination.

The present invention also provides peptides of the following subgeneric formulae VIII and IX, wherein the variables are as described in formula VII:

(D-R-S-W-T-N-O-(Xₙ-Y_{z}-Zₘ)ₛ)ᵣ VIII

((Xₙ-Y_{z}-Zₘ)ₛ-O′-N′-T′-W′-S′-R′D′)ᵣ IX

In some embodiments, the D may be placed in the position of W and W may be placed in the position of D in formula VII to yield the following:

(W-R-S-D-T-N-O-(Xₙ-Y_{z}-Zₘ)ₛ-O′-N′-T′-W′-S′-R′-D′)ᵣ X

In some embodiments, the D′ may be placed in the position of W′, and W′ may be placed in the position of D′ in formula VII to yield the following:

(D-R-S-W-T-N-O-(Xₙ-Y_{z}-Zₘ)ₛ-O′-N′-T′-D′-S′-R′-W′)ᵣ XI

In some embodiments, the D may be placed in the position of W and W may be placed in the position of D in formula VII and D′ may be placed in the position of W′, and W′ may be placed in the position of D′ in formula VII to yield the following:

(W-R-S-D-T-N-O-(Xₙ-Y_{z}-Zₘ)ₛ-O′-N′-T′-D′-S′-R′-W′)ᵣ XII

The present invention also provides peptides of the following formulae:

(W-R-S-D-T-N-O-(Xₙ-Y_{z}-Zₘ)ₛ)ᵣ XIII

((Xₙ-Y_{z}-Zₘ)ₛ-O′-N′-T′-D′-S′-R′-W′)ᵣ XIV

(D-R-S-T-N-O-(Xₙ-Y_{z}-Zₘ)ₛ)ᵣ XV

D-W XVI

W-D XVII

(Xₙ-Y_{z}-Zₘ)ₛ XVIII

The present invention also includes compositions comprising combinations of individual peptides of the present invention in an acceptable carrier. For example, a mixture of D, W, and (Xₙ-Y_{z}-Zₘ)ₛ may be made in an acceptable carrier. These peptides are as defined above and may be labeled or unlabelled. It is to be understood that a mixture of peptides, such as D, W, and (Xₙ-Y_{z}-Zₘ)ₛ may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity.

It is to be understood that the letters in the generic formulae I through XVIII or in components thereof are defined by the text that follows each letter and do not designate an individual amino acid.

It is to be understood that in some embodiments, one or more of the amino acids of the peptides of the present invention are D amino acids. In one embodiment, the N-terminal amino acid, the C-terminal amino acid or both are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

The N and/or C-terminal amino acids may also be modified by amidation, acetylation or other modifications known to one of ordinary skill in the art. The peptides of the present invention may optionally be acetylated at the N-terminus or the C-terminus using techniques known to one of ordinary skill in the art. The peptides of the present invention may optionally be amidated at the N-terminus or the C-terminus using techniques known to one of ordinary skill in the art. In one embodiment, the peptides of the present invention are acetylated at the N-terminus, amidated at the C-terminus, or both acetylated at the N-terminus and amidated at the C-terminus. In some embodiments, the peptides of the present invention may have both an acetylated N-terminus and a carboxy terminal amide. In the present application, when a peptide is acetylated on an N or C terminus, the letters Ac are indicated. In the present application, when a peptide is amidated on an N or C terminus, the designation NH₂ is employed.

The present invention also includes compositions comprising one or more individual peptides of the present invention in an acceptable carrier. These peptides are as defined above and may be labeled or unlabelled. It is to be understood that a mixture of peptides, may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity.

Accordingly, it is an object of the present invention to provide novel peptides.

Accordingly, it is an object of the present invention to provide novel peptides that facilitate lipid efflux.

Yet another object of the present invention is to provide novel peptides that facilitate lipid efflux and possess anti-inflammatory biological activity.

Still another object of the present invention is to provide novel peptides that facilitate lipid efflux and stimulate LCAT activity.

Yet another object of the present invention is to provide novel peptides that facilitate lipid efflux, possess anti-inflammatory biological activity, and stimulate LCAT activity.

It is another object of the present invention to provide new methods for visualizing plaque using labeled peptides of the present invention.

It is yet another object of the present invention to provide new methods for the treatment of atherosclerosis, cardiovascular disease and cerebrovascular disease in an animal or a human by administering pharmaceutical compositions comprising one or more peptides of the present invention with a pharmaceutically acceptable carrier, or on a medical device.

These and other objects, features and advantages of the present invention will become apparent after a review of the following detailed description of the disclosed embodiments and claims.

### BRIEF DESCRIPTION OF THE FIGURES

FIG. 1 shows the amino acid sequence of ApoA-I (SEQ ID NO: 36).

FIG. 2 is a schematic illustration of the statistically significant, anti-inflammatory effects of ApoA-I (SEQ ID NO: 36) and peptide 1 (SEQ ID NO: 624), (each at 20 ug/ml) to decrease PMA (1 uM) induced expression of CD11b in human monocytes.

FIG. 3 is a schematic illustration of the statistically significant, anti-inflammatory effects of ApoAI and peptide 6 (SEQ ID NO: 155), (each at 20 ug/ml) to decrease PMA (1 uM) induced expression of CD11b in human monocytes.

FIG. 4 is a schematic illustration of the dose dependent stimulation of cholesterol efflux from the cells containing the ABCAl pathway 1=SEQ ID NO: 624, 2=SEQ ID NO: 121, 3=SEQ ID NO: 121, 4=SEQ ID NO: 130, 5=SEQ ID NO: 624.

### DETAILED DESCRIPTION OF THE PRESENT INVENTION

The present invention provides novel peptides. The present invention solves the problems described above by providing novel peptide compositions with functional domains. In some embodiments, these novel peptide compositions promote lipid efflux. In some embodiments, these novel peptide compositions promote lipid efflux and have anti-inflammatory properties. In other embodiments, these novel peptide compositions promote lipid efflux and have one or more anti-inflammatory domains. In yet other embodiments, these novel peptide compositions promote lipid efflux and have one or more domains that affect LCAT activity. In several embodiments, these novel peptide compositions promote lipid efflux and have one or more anti-inflammatory domains and one or more domain that affects LCAT activity.

Any of the peptides of the present invention may optionally be acetylated at the N-terminus or the C-terminus using techniques known to one of ordinary skill in the art. The peptides of the present invention may optionally be amidated at the N-terminus or the C-terminus using techniques known to one of ordinary skill in the art. In one embodiment, the peptides of the present invention are acetylated at the N-terminus, amidated at the C-terminus, or both acetylated at the N-terminus and amidated at the C-terminus. In some embodiments, the peptides of the present invention may have both an acetylated N-terminus and a carboxy terminal amide. In the present application, when a peptide is acetylated on an N or C terminus, the letters Ac are indicated. In the present application, when a peptide is amidated on an N or C terminus, the designation NH₂ is employed.

One or more of these peptides may be combined with an acceptable carrier and administered as compositions to individuals in order to provide lipid efflux and anti-inflammatory activities. These compositions may be administered to treat dyslipidemic and vascular disorders or to delay or prevent the onset or progression of dyslipidemic and vascular disorders. In one embodiment, these compositions may be administered to treat atherosclerosis or to delay or prevent its onset or progression. These novel peptide compositions may be labeled and used in a variety of applications including the visualization of plaque in vessels. These novel peptide compositions also display low toxicity.

### I. Abbreviations

ABCAl: ATP-binding cassette transporter Al

apoA-I: apolipoprotein A-I

DMPC: dimyristoyl phosphatidyl choline

HDL: high-density lipoprotein

HPLC: high-pressure liquid chromatography

LDL: low-density lipoprotein

RBC: red blood cell

### II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

In order to facilitate review of the various embodiments of this disclosure, the following explanations of specific terms are provided:

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Animal: Living multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows.

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term “antibody” includes intact immunoglobulins as well as a number of well-characterized fragments. For instance, Fabs, Fvs, and single-chain Fvs (SCFvs) that bind to target protein (or epitope within a protein or fusion protein) would also be specific binding agents for that protein (or epitope). These antibody fragments are as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, e.g., Harlow and Lane, Using Antibodies: A Laboratory Manual, CSHL, New York, 1999).

Antibodies for use in the methods and compositions of this disclosure can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described in Harlow and Lane, Using Antibodies: A Laboratory Manual, CSHL, New York, 1999.

Domain: A domain of a protein is a part of a protein that shares common structural, physiochemical and functional features; for example hydrophobic, polar, globular, helical domains or properties, for example a DNA binding domain, an ATP binding domain, an anti-inflammatory domain, an LCAT activating domain and the like. Some peptides of the present invention possess a domain or domains that have more than one functional feature, for example both lipid efflux activity and anti-inflammatory activity.

Dyslipidemic disorder: A disorder associated with any altered amount of any or all of the lipids or lipoproteins in the blood. Dyslipidemic disorders include, for example, hyperlipidemia, hyperlipoproteinemia, hypercholesterolemia, hypertriglyceridemia, HDL deficiency, apoA-I deficiency, and cardiovascular disease (e.g., coronary artery disease, atherosclerosis and restenosis).

Efflux: The process of flowing out. As applied to the results described herein, lipid efflux refers to a process whereby lipid, such as cholesterol and phospholipid, is complexed with an acceptor, such as an apolipoprotein or apolipoprotein peptide mimetic, or a peptide of the present invention and removed from vesicles or cells. “ABCAl-dependent lipid efflux” (or lipid efflux by an “ABCAl-dependent pathway”) refers to a process whereby apolipoproteins, synthetic peptide mimetics of apolipoproteins, or a peptide of the present invention, bind to a cell and efflux lipid from the cell by a process that is facilitated by the ABCAl transporter.

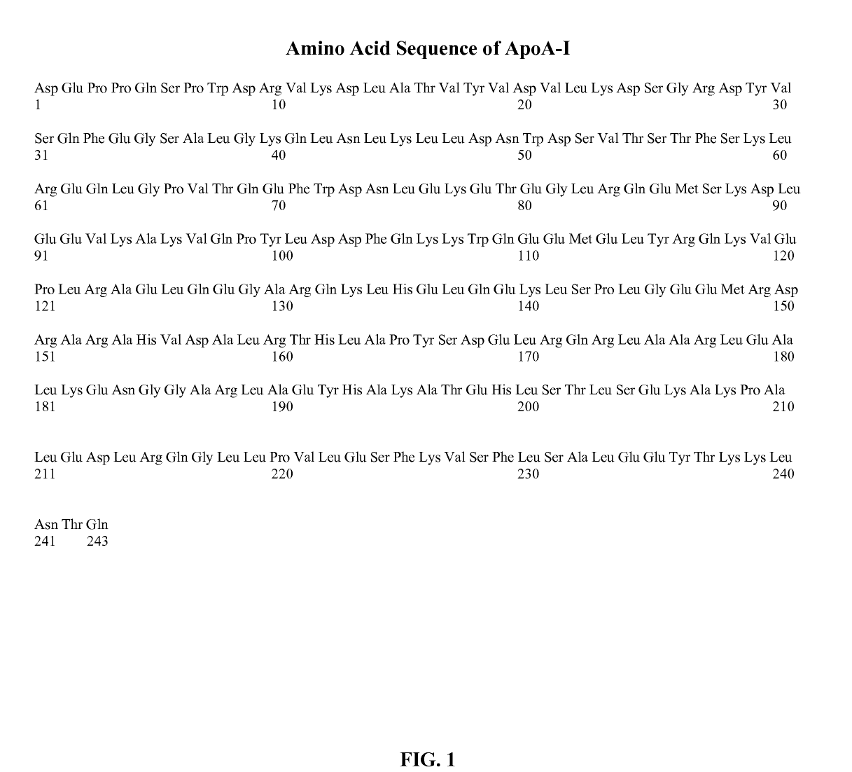

Helix: The molecular conformation of a spiral nature, generated by regularly repeating rotations around the backbone bonds of a macromolecule. Helices 5, 6 and 8 of ApoA-I are as follows wherein each helix number is followed by the amino acid residues associated with that helix: 5:145-162; 6:167-184; 8:222-239. FIG. 1 shows the amino acid sequence of ApoA-I (SEQ ID NO: 36).

Hydrophobic: A hydrophobic (or lipophilic) group is electrically neutral and nonpolar, and thus prefers other neutral and nonpolar solvents or molecular environments. Examples of hydrophobic molecules include alkanes, oils and fats.

Hydrophilic: A hydrophilic (or lipophobic) group is electrically polarized and capable of H-bonding, enabling it to dissolve more readily in water than in oil or other “non-polar” solvents.

Inhibiting or treating a disease: Inhibiting the full development of a disease, disorder or condition, for example, in a subject who is at risk for a disease such as atherosclerosis and cardiovascular disease. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

Isolated/purified: An “isolated” or “purified” biological component (such as a nucleic acid, peptide or protein) has been substantially separated, produced apart from, or purified away from other biological components in the cell of the organism in which the component naturally occurs, that is, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a host cell as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell. Preferably, a preparation is purified such that the biological component represents at least 50%, such as at least 70%, at least 90%, at least 95%, or greater of the total biological component content of the preparation.

Label: A detectable compound or composition that is conjugated directly or indirectly to another molecule to facilitate detection of that molecule. Specific, non-limiting examples of labels include fluorescent tags, calorimetric labels, dyes, beads, enzymatic linkages, radiodense materials, and radioactive isotopes.

Linker: A molecule that joins two other molecules, either covalently, or through ionic, van der Waals or hydrogen bonds.

Lipid: A class of water-insoluble, or partially water insoluble, oily or greasy organic substances, that are extractable from cells and tissues by nonpolar solvents, such as chloroform or ether. Types of lipids include triglycerides (e.g., natural fats and oils composed of glycerin and fatty acid chains), glycolipids, phospholipids (e.g., phosphatidylethanolamine, phosphatidylcholine, phosphatidylserine, and phosphatidylinositol), sphingolipids (e.g., sphingomyelin, cerebrosides and gangliosides), and sterols (e.g., cholesterol).

Lipid affinity: A measurement of the relative binding affinity of an amphipathic α-helix for lipids. In some embodiments, the lipid affinity of an amphipathic a-helix is determined by one or more functional tests. Specific, non-limiting examples of functional tests include: retention time on reverse phase HPLC, surface monolayer exclusion pressure (Palgunachari et al., Arterioscler. Thromb. Vasc. Biol. 16:328-338, 1996), binding affinity to phospholipid vesicles (Palgunachari et al., Arterioscler. Thromb. Vasc. Biol. 16:328-338, 1996), and DMPC vesicle solubilization (Remaley et al., J. Lipid Res. 44:828-836, 2003).

Further non-limiting examples of alternative methods of calculating the lipid affinity of an amphipathic a-helix include: total hydrophobic moment, total peptide hydrophobicity, total peptide hydrophobicity per residue, hydrophobicity of amino acids on the hydrophobic face, hydrophobicity per residue of amino acids on the hydrophobic face, and calculated lipid affinity based on predicted peptide penetration into phospholipid bilayers (Palgunachari et al., Arterioscler. Thromb. Vasc. Biol. 16:328-338, 1996).

Non-Cytotoxic:

A non-cytotoxic compound is one that does not substantially affect the viability or growth characteristics of a cell at a dosage normally used to treat the cell or a subject. Furthermore, the percentage of cells releasing intracellular contents, such as LDH or hemoglobin, is low (e.g., about 10% or less) in cells treated with a non-cytotoxic compound. Lipid efflux from a cell that occurs by a non-cytotoxic compound results in the removal of lipid from a cell by a process that maintains the overall integrity of the cell membrane and does not lead to significant cell toxicity.

Non-Polar: A non-polar compound is one that does not have concentrations of positive or negative electric charge. Non-polar compounds, such as, for example, oil, are not well soluble in water.

Peptide:

A polymer in which the monomers are amino acid residues which are joined together through amide bonds. When the amino acids are alpha-amino acids, either the L-optical isomer or the D-optical isomer can be used. The amino acid sequences disclosed herein are shown using three letter codes for amino acids, as defined in 37 C.F.R. 1.822 and as commonly known to one of ordinary skill in the art. When the three letter designation for an amino acid, for example Ser for serine is shown in upper case, SER, the serine is a D amino acid. The terms “peptide” or “polypeptide” as used herein are intended to encompass any amino acid sequence and include modified sequences such as glycoproteins. The term “peptide” is specifically intended to cover naturally occurring peptides, as well as those which are recombinantly or synthetically produced. The term “residue” or “amino acid residue” includes reference to an amino acid that is incorporated into a peptide, polypeptide, or protein. As known to one of skill in the art, the peptides presented herein are read from the N to the C terminus i.e., from left to right. Accordingly, the N terminal amino acid in Leu Glu Lys is Leu and the C-terminal amino acid is Lys. Peptides of the present invention include conservatively substituted peptides, wherein these conservative substitutions occur at 1%, 3%, 5%, 7%, 10%, 15%, 20%, 25%, 30%, 40%, or 50% of the amino acid residues. Peptides of the present invention include peptides that are homologous at 50%, 60%, 70%, 80%, 90%, 95%, 97%, 98%, 99% of the entire sequence of the peptide.

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more peptides or peptide analogs and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospholipid: A phospholipid consists of a water-soluble polar head, linked to two water-insoluble non-polar tails (by a negatively charged phosphate group). Both tails consist of a fatty acid, each about 14 to about 24 carbon groups long. When placed in an aqueous environment, phospholipids form a bilayer or micelle, where the hydrophobic tails line up against each other. This forms a membrane with hydrophilic heads on both sides. A phospholipid is a lipid that is a primary component of animal cell membranes.

Polar:

A polar molecule is one in which the centers of positive and negative charge distribution do not converge. Polar molecules are characterized by a dipole moment, which measures their polarity, and are soluble in other polar compounds and virtually insoluble in nonpolar compounds.

Recombinant nucleic acid: A sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques such as those described in Sambrook et al. (ed.), Molecular Cloning: A Laboratory Manual, 2^{nd} ed., vol. 1-3, Cold Spring Harbor Laboratory Press, Cold Spring Harbor, N.Y., 1989. The term recombinant includes nucleic acids that have been altered solely by addition, substitution, or deletion of a portion of the nucleic acid.

Therapeutically effective amount: A quantity of a specified agent sufficient to achieve a desired effect in a subject being treated with that agent. For example, this can be the amount of a peptide or peptide analog useful in preventing, ameliorating, and/or treating a dyslipidemic disorder (e.g., atherosclerosis) in a subject. Ideally, a therapeutically effective amount of an agent is an amount sufficient to prevent, ameliorate, and/or treat a dyslipidemic disorder (e.g., atherosclerosis) in a subject without causing a substantial cytotoxic effect (e.g., membrane microsolubilization) in the subject. The effective amount of an agent useful for preventing, ameliorating, and/or treating a dyslipidemic disorder (e.g., atherosclerosis) in a subject will be dependent on the subject being treated, the severity of the disorder, and the manner of administration of the therapeutic composition.

Transformed:

A “transformed” cell is a cell into which has been introduced a nucleic acid molecule by molecular biology techniques. The term encompasses all techniques by which a nucleic acid molecule might be introduced into such a cell, including transfection with viral vectors, transformation with plasmid vectors, and introduction of naked DNA by electroporation, lipofection, and particle gun acceleration.

### III. Peptides of the Present Invention and Analogs Thereof

In one embodiment, the peptides of the present invention are described by the following generic formula I:

(A-B-C)ₙ I

In one embodiment, A is helix 5 of ApoA-I and is SEQ ID NO: 1 Gly Glu Glu Met Arg Asp Arg Ala Arg Ala His Val Asp Ala Leu Arg Thr His, or a conservative substitution thereof. These amino acids may also appear in reverse orientation as in SEQ ID NO: 2 His Thr Arg Leu Ala Asp Val His Ala Arg Ala Arg Asp Arg Met Glu Glu Gly.

In another embodiment, A is helix 6 of ApoA-I and is SEQ ID NO: 3 Ser Asp Glu Leu Arg Gln Arg Leu Ala Ala Arg Leu Glu Ala Leu Lys Glu Asn, or a conservative substitution thereof. These amino acids may also appear in reverse orientation as in SEQ ID NO: 4 Asn Glu Lys Leu Ala Glu Leu Arg Ala Ala Leu Arg Gln Arg Leu Glu Asp Ser.

In one embodiment, A is a modified form of helix 8 of ApoA-I, also called 8′ herein, and is SEQ ID NO: 5 Leu Glu Ser Ala Lys Val Ser Ala Leu Ser Ala Leu Glu Glu Ala Thr Lys Lys, or a conservative substitution thereof. These amino acids may also appear in reverse orientation such that Lys is at the N-terminus and Leu is at the C-terminus as in SEQ ID NO: 6 Lys Lys Thr Ala Glu Glu Leu Ala Ser Leu Ala Ser Val Lys Ala Ser Glu Leu. This modification of helix 8 involves substitutions at positions 4 (Phe to Ala), 8 (Phe to Ala) and 15 (Tyr to Ala). It is to be understood that the present invention encompasses other amino acid substitutions at these locations. At positions 4 and 8, Phe may be substituted with Val, Leu, Gly, Thr, Ser or gamma aminobutyric acid (GABA). At position 15, Tyr may be substituted with Val, Leu, Gly, Thr, Ser or GABA. While not wanting to be bound by the following statement, it is believed that A, the modified form of helix 8 of ApoA-I, has a lower lipid affinity than C, the unmodified form of helix 8 of ApoA-I.

In one embodiment, B is Pro, SEQ ID NO: 7 Lys Leu Ser Pro Leu, SEQ ID NO: 8 Leu Ser Pro Leu, or SEQ ID NO: 9 Ser Pro Leu, or a conservative substitution thereof. These amino acids may also appear in reverse orientation for example as in SEQ ID NO: Leu Pro Ser Leu Lys, SEQ ID NO: 11 Leu Pro Ser Leu, and SEQ ID NO: 12 Leu Pro Ser.

In one embodiment, C is helix 8 of ApoA-I and is SEQ ID NO: 13 Leu Glu Ser Phe Lys Val Ser Phe Leu Ser Ala Leu Glu Glu Tyr Thr Lys Lys, or a conservative substitution thereof. These amino acids may also appear in reverse orientation such that Lys is at the N-terminus and Leu is at the C-terminus as SEQ ID NO: 14 Lys Lys Thr Tyr Glu Glu Leu Ala Ser Leu Phe Ser Val Lys Phe Ser Glu Leu. It is to be understood that A and C may be switched in location as in C-B-A.

Specific embodiments of peptides represented by generic formula I are:

### Wherein A is 5 and C is 8

SEQ ID NO: 37

Gly Glu Glu Met Arg Asp Arg Ala Arg Ala His Val
Asp Ala Leu Arg Thr His Lys Leu Ser Pro Leu Leu
Glu Ser Phe Lys Val Ser Phe Leu Ser Ala Leu Glu
Glu Tyr Thr Lys Lys;

SEQ ID NO: 38

Gly Glu Glu Met Arg Asp Arg Ala Arg Ala His Val
Asp Ala Leu Arg Thr His Pro Leu Glu Ser Phe Lys
Val Ser Phe Leu Ser Ala Glu Glu Tyr Thr Lys Lys;

### Wherein A is 6 and C is 8

SEQ ID NO: 39

Ser Asp Glu Leu Arg Gln Arg Leu Ala Ala Leu Glu
Ala Leu Lys Glu Asn Lys Leu Ser Pro Leu Leu Gln
Ser Phe Lys Val Ser Phe Leu Ser Ala Leu Glu Glu
Tyr Thr Lys Lys;

SEQ ID NO: 40

Ser Asp Glu Leu Arg Gln Arg Leu Ala Ala Arg Leu
Glu Ala Leu Lys Glu Asn Pro Leu Glu Ser Phe Lys
Val Ser Phe Leu Ser Ala Leu Glu Glu Tyr Thr Lys
Lys;

### Wherein A is 8′ and C is 8

SEQ ID NO: 41

Leu Glu Ser Ala Lys Val Ser Ala Leu Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys;

SEQ ID NO: 42

Leu Glu Ser Ala Lys Val Ser Ala Leu Glu Glu Ala
Thr Lys Lys Pro Leu Glu Ser Phe Lys Val Ser Phe
Leu Ser Ala Leu Glu Glu Tyr Thr Lys Lys;

SEQ ID NO: 43

Leu Glu Ser Ala Lys Val Ser Ala Leu Ser Ala Leu
Glu Glu Ala Thr Lys Lys Lys Leu Ser Pro Leu Lys
Lys Thr Tyr Glu Glu Leu Ala Ser Leu Phe Ser Val
Lys Phe Ser Glu Leu;

SEQ ID NO: 44

Leu Glu Ser Ala Lys Val Ser Ala Leu Ser Ala Leu
Glu Glu Ala Thr Lys Lys Pro Lys Lys Thr Tyr Glu
Glu Leu Ala Ser Leu Phe Ser Val Lys Phe Ser Glu
Leu;

SEQ ID NO: 45

Lys Lys Thr Ala Glu Glu Leu Ala Ser Leu Ala Ser
Val Lys Ala Ser Glu Leu Lys Leu Ser Pro Leu Lys
Lys Thr Tyr Glu Glu Leu Ala Ser Leu Phe Ser Val
Lys Phe Ser Glu Leu;

SEQ ID NO: 46

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu;

SEQ ID NO: 47

Lys Lys Thr Ala Glu Glu Leu Ala Ser Leu Ala Ser
Val Lys Ala Ser Glu Leu Lys Leu Ser Pro Leu Leu
Glu Ser Phe Lys Val Ser Phe Leu Ser Ala Leu Glu
Glu Tyr Thr Lys Lys;

SEQ ID NO: 48

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys;

SEQ ID NO: 49

Leu Glu Ser Ala Lys Val Ser Ala Leu Ser Ala Leu
Glu Glu Ala Thr Lys Lys Leu Pro Ser Leu Lys Leu
Glu Ser Phe Lys Val Ser Phe Leu Ser Ala Leu Glu
Glu Tyr Thr Lys Lys;

SEQ ID NO: 50

Leu Glu Ser Ala Lys Val Ser Ala Leu Ser Ala Leu
Glu Glu Ala Thr Lys Pro Lys Leu Glu Ser Phe Lys
Val Ser Phe Leu Ser Ala Leu Glu Glu Tyr Thr Lys
Lys;

SEQ ID NO: 51

Leu Glu Ser Ala Lys Val Ser Ala Leu Ser Ala Leu
Glu Glu Ala Thr Lys Lys Leu Pro Ser Leu Lys Lys
Lys Thr Tyr Glu Glu Leu Ala Ser Leu Phe Ser Val
Lys Phe Ser Glu Leu;

SEQ ID NO: 52

Leu Glu Ser Ala Lys Val Ser Ala Leu Ser Ala Leu
Glu Glu Ala Thr Lys Pro Lys Lys Lys Thr Tyr Glu
Glu Leu Ala Ser Leu Phe Ser Val Lys Phe Ser Glu
Leu;

SEQ ID NO: 53

Lys Lys Thr Ala Glu Glu Leu Ala Ser Leu Ala Ser
Val Lys Ala Ser Glu Leu Leu Pro Ser Leu Lys Lys
Lys Thr Tyr Glu Glu Leu Ala Ser Leu Phe Ser Val
Lys Phe Ser Glu Leu;

SEQ ID NO: 54

Lys Lys Thr Ala Glu Glu Leu Ala Ser Leu Ala Ser
Val Lys Ala Ser Glu Leu Leu Pro Ser Leu Lys Leu
Glu Ser Phe Lys Val Ser Phe Leu Ser Ala Leu Glu
Glu Tyr Thr Lys Lys;
and,

SEQ ID NO: 55

Lys Lys Thr Ala Glu Glu Leu Ala Ser Leu Ala Ser
Val Lys Ala Ser Glu Leu Pro Glu Ser Phe Lys Val
Ser Phe Leu Ser Ala Leu Glu Glu Tyr Thr Lys Lys;

### Wherein A is 8 and C is 8′

SEQ ID NO: 56

Leu Glu Ser Phe Leu Val Ser Phe Lys Ser Ala Leu
Glu Glu Tyr Phe Glu Lys Lys Leu Ser Pro Leu Leu
Glu Ser Ala Ala Val Ser Ala Lys Ser Ala Leu Glu
Glu Tyr Ala Glu Lys;

In a further embodiment, peptides of the present invention are described by the following subgeneric formula II, in which one or more additional elements indicated as variables D, E, F and W, are added to formula I to make subgeneric formula II.

D-E-(A-B-C)ₙ-F-W II

(A-B-C)ₙ are as described in formula I above.

D is absent or present and is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

E is absent or present and is a group linking D and A and is Pro, SEQ ID NO: 10 Leu Pro Ser Leu Lys, or a conservative substitution thereof, provided that E is present only when D is present. These amino acids may also appear in reverse orientation as in SEQ ID NO: 7 Lys Leu Ser Pro Leu. When D is absent, E is absent.

F is absent or present and is a group linking C and W and is Pro, SEQ ID NO: 19 Ala Leu Ser Pro Leu, or a conservative substitution thereof, provided that F is present only when W is present. These amino acids may also appear in reverse orientation as in SEQ ID NO: 20 Leu Pro Ser Leu Ala. When W is absent, F is absent.

W is absent or present and is a peptide as defined in the present specification. In one embodiment, W is a peptide selected from the group consisting of SEQ ID NO: 21 Trp Arg Trp Trp Trp Trp), or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

As stated above, in generic formula II, variables D or W may be absent or present. In one embodiment, D is present and W is absent. In another embodiment, W is present and D is absent. In another embodiment, both D and W are present.

Specific embodiments of peptides represented by generic formula II are:

### Wherein A is 5 or 6 and C is 8

SEQ ID NO: 57

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO: 58

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Lys Leu Ser Pro
Leu Leu Glu Ser Phe Lys Val Ser Phe Leu Ser Ala
Leu Glu Glu Tyr Thr Lys Lys;

SEQ ID NO: 59

Gly Glu Glu Met Arg Asp Arg Ala Arg Ala His Val
Asp Ala Leu Arg Thr His Lys Leu Ser Pro Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO: 60

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO: 61

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Lys Leu Ser Pro
Leu Leu Glu Ser Phe Lys Val Ser Phe Leu Ser Ala
Leu Glu Glu Tyr Thr Lys Lys;

SEQ ID NO: 62

Ser Asp Glu Leu Arg Gln Arg Leu Ala Ala Arg Leu
Glu Ala Leu Lys Glu Asn Lys Leu Ser Pro Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO: 63

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Pro Leu Glu Ser
Phe Lys Val Ser Phe Leu Ser Ala Leu Glu Glu Tyr
Thr Lys Lys Ala Leu Ser Pro Leu Trp Arg Trp Trp
Trp Trp;

SEQ ID NO: 64

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Pro Leu Glu Ser
Phe Lys Val Ser Phe Leu Ser Ala Leu Glu Glu Tyr
Thr Lys Lys;

SEQ ID NO: 65

Gly Glu Glu Met Arg Asp Arg Ala Arg Ala His Val
Asp Ala Leu Arg Thr His Pro Leu Glu Ser Phe Lys
Val Ser Phe Leu Ser Ala Leu Glu Glu Tyr Thr Lys
Lys Ala Leu Ser Pro Leu Trp Arg Trp Trp Trp Trp;

SEQ ID NO: 66

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Pro Leu Glu Ser
Phe Lys Val Ser Phe Leu Ser Ala Leu Glu Glu Tyr
Thr Lys Lys Ala Leu Ser Pro Leu Trp Arg Trp Trp
Trp Trp;

SEQ ID NO: 67

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Pro Leu Glu Ser
Phe Lys Val Ser Phe Leu Ser Ala Leu Glu Glu Tyr
Thr Lys Lys;
or,

SEQ ID NO: 68

Ser Asp Glu Leu Arg Gln Arg Leu Ala Ala Arg Leu
Glu Ala Leu Lys Glu Asn Pro Leu Glu Ser Phe Lys
Val Ser Phe Leu Ser Ala Leu Glu Glu Tyr Thr Lys
Lys Ala Leu Ser Pro Leu Trp Arg Trp Trp Trp Trp

### Wherein A is 8′ and C is 8

SEQ ID NO: 69

Pro Arg gly ser val leu val thr leu pro ser leu
Lys Leu Glu Ser Ala Lys Val Ser Ala Leu Ser Ala
Leu Glu Glu Ala Thr Lys Lys Lys Leu Ser Pro Leu
Leu Glu Ser peh Lys Val Ser Phe Leu Ser Ala Leu
Glu Glu Tyr Thr Lys Lys Ala Leu Ser Pro Leu Trp
Arg Trp Trp Trp Trp;

SEQ ID NO: 70

Pro Arg Gly Gly Ser Val Leu Val Thr Leu Pro Ser
Lys Lys Leu Glu Ser Ala Lys Val Ser Ala Leu Ser
Ala Leu Glu Glu Ala Thr Lys Lys Lys Leu Ser Pro
Leu Leu Gly Ser Phe Lys Val Ser Phe Leu Ser Ala
Leu Gly Gly Tyr Thr Lys Lys;

SEQ ID NO: 71

Leu Glu Ser Ala Lys Val Ser Ala Leu Ser Ala Leu
Glu Glu Ala Thr Lys Lys Lys Leu Ser Pro Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO: 72

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO: 73

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Leu Glu Ser Phe Lys Val Ser Phe Leu Ser Ala
Leu Glu Glu Tyr Thr Lys Lys;

SEQ ID NO: 74

Lys Lys Thr Ala Glu Glu Leu Ala Ser Leu Ala Ser
Val Lys Ala Ser Glu Leu Lys Leu Ser Pro Leu Leu
Gly Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO: 75

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Lys Lys Thr Tyr Glu Glu Leu Ala Ser Leu Phe
Ser Val lus Phe Ser Glu Leu Ala Leu Ser Pro Leu
Trp Arg Trp Trp Trp Trp;

SEQ ID NO: 76

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Lys Lys Thr Tyr Glu Glu Leu Ala Ser Leu Phe
Ser Val Lys Phe Ser Glu Leu;

SEQ ID NO: 77

Lys Lys Thr Ala Glu Glu Leu Ala Ser Leu Ala Ser
Val Lys Ala Ser Glu Leu Lys Leu Ser Pro Leu Lys
Lys Thr Tyr Glu Glu Leu Ala Ser Leu Phe Ser Val
Lys Phe Ser Glu Leu Trp Arg Trp Trp Trp Trp;

SEQ ID NO: 78

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Pro Leu Glu Ser
Phe Lys Val Ser Phe Leu Ser Ala Leu Glu Glu Tyr
Thr Lys Lys Ala Leu Ser Pro Leu Trp Arg Trp Trp
Trp Trp;

SEQ ID NO: 79

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Pro Leu Glu Ser
Phe Lys Val Ser Phe Leu Ser Ala Leu Glu Glu Tyr
Thr Lys Lys;

SEQ ID NO: 80

Leu Glu Ser Ala Lys Val Ser Ala Leu Ser Ala Leu
Glu Glu Ala Thr Lys Lys Pro Leu Glu Ser Phe Lys
Val Ser Phe Leu Ser Ala Leu Glu Glu Tyr Thr Lys
Lys Ala Leu Ser Pro Leu Trp Arg Trp Trp Trp Trp;

SEQ ID NO: 81

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys Ala Leu Ser Pro Leu Trp Arg Trp Trp
Trp Trp;

SEQ ID NO: 82

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys;

SEQ ID NO: 83

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Ala Leu Ser Pro Leu Trp Arg Trp Trp Trp Trp;

SEQ ID NO: 84

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Pro Lys Thr
Tyr Glu Glu Leu Ala Ser Leu Phe Ser Val Lys Phe
Ser Glu Leu Ala Leu Ser Pro Leu Trp Arg Trp Trp
Trp Trp;

SEQ ID NO: 85

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu;

SEQ ID NO: 86

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Trp Arg Trp Trp Trp Trp;
and,

SEQ ID NO: 87

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu Ala Leu Ser Pro Leu Trp Arg Trp Trp
Trp Trp;

In yet another embodiment, W and D as described in formula III may be switched in location.

W-E-(A-B-C)ₙ-F-D III

Specific embodiments of peptides represented by generic formula III are as follows:

### Wherein A is 5 or 6 and C is 8

SEQ ID NO: 88

Trp Arg Trp Trp Trp Trp Leu Pro Ser Leu Lys Gly
Glu Glu Met Arg Asp Arg Ala Arg Ala His Val Asp
Ala Leu Arg Thr His Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO: 89

Gly Glu Glu Met Arg Asp Arg Ala Arg Ala His Val
Asp Ala Leu Arg Thr His Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO: 90

Trp Arg Trp Trp Trp Trp Leu Pro Ser Leu Lys Gly
Glu Glu Met Arg Asp Arg Ala Arg Ala His Val Asp
Ala Leu Arg Thr His Lys Leu Ser Pro Leu Leu Glu
Ser Phe Lys Val Ser Phe Leu Ser Ala Leu Glu Glu
Tyr Thr Lys Lys;

SEQ ID NO: 91

Trp Arg Trp Trp Trp Trp Leu Pro Ser Leu Lys Ser
Asp Glu Leu Arg Gln Arg Leu Ala Ala Arg Leu Glu
Ala Leu Lys Glu Asn Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO: 92

Ser Asp Glu Leu Arg Gln Arg Leu Ala Ala Arg Leu
Glu Ala Leu Lys Glu Asn Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO: 93

Trp Arg Trp Trp Trp Trp Leu Pro Ser Leu Lys Ser
Asp Glu Leu Arg Gln Arg Leu Ala Ala Arg Leu Glu
Ala Leu Lys Glu Asn Lys Leu Ser Pro Leu Leu Glu
Ser Phe Lys Val Ser Phe Leu Ser Ala Leu Glu Glu
Tyr Thr Lys Lys;

SEQ ID NO: 94

Trp Arg Trp Trp Trp Trp Leu Pro Ser Leu Lys Gly
Glu Glu Met Arg Asp Arg Ala Arg Ala His Val Asp
Ala Leu Arg Thr His Pro Leu Glu Ser Phe Lys Val
Ser Phe Leu Ser Ala Leu Glu Glu Tyr Thr Lys Lys
Ala Leu Ser Pro Leu Pro Arg Gly Gly Ser Val Leu
Val Thr;

SEQ ID NO: 95

Gly Glu Glu Met Arg Asp Arg Ala Arg Ala His Val
Asp Ala Leu Arg Thr His Pro Leu Glu Ser Phe Lys
Val Ser Phe Leu Ser Ala Leu Glu Glu Tyr Thr Lys
Lys Ala Leu Ser Pro Leu Pro Arg Gly Gly Ser Val
Leu Val Thr;

SEQ ID NO: 96

Trp Arg Trp Trp Trp Trp Leu Pro Ser Leu Lys Gly
Glu Glu Met Arg Asp Arg Ala Arg Ala His Val Asp
Ala Leu Arg Thr His Pro Leu Glu Ser Phe Lys Val
Ser Phe Leu Ser Ala Leu Glu Glu Tyr Thr Lys Lys;

SEQ ID NO: 97

Trp Arg Trp Trp Trp Trp Leu Pro Ser Leu Lys Ser
Asp Glu Leu Arg Gln Arg Leu Ala Ala Arg Leu Glu
Ala Leu Lys Glu Asn Pro Leu Glu Ser Phe Lys Val
Ser Phe Leu Ser Ala Leu Glu Glu Tyr Thr Lys Lys
Ala Leu Ser Pro Leu Pro Arg Gly Gly Ser Val Leu
Val Thr;

SEQ ID NO: 98

Ser Asp Glu Leu Arg Gln Arg Leu Ala Ala Arg Leu
Glu Ala Leu Lys Glu Asn Pro Leu Glu Ser Phe Lys
Val Ser Phe Leu Ser Ala Leu Glu Glu Tyr Thr Lys
Lys Ala Leu Ser Pro Leu Pro Arg Gly Gly Ser Val
Leu Val Thr;
or,

SEQ ID NO: 99

Trp Arg Trp Trp Trp Trp Leu Pro Ser Leu Lys Ser
Asp Glu Leu Arg Gln Arg Leu Ala Ala Arg Leu Glu
Ala Leu Lys Glu Asn Pro Leu Glu Ser Phe Lys Val
Ser Phe Leu Ser Ala Leu Glu Glu Tyr Thr Lys Lys.

### Wherein A is 8′ and C is 8

SEQ ID NO: 100

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO: 101

Leu Glu Ser Ala Lys Val Ser Ala Leu Ser Ala Leu
Glu Glu Ala Thr Lys Lys Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO: 102

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Lys Leu Ser Pro Leu Leu Glu
Ser Phe Lys Val Ser Phe Leu Ser Ala Leu Glu Glu
Tyr Thr Lys Lys;

SEQ ID NO: 103

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO: 104

Lys Lys Thr Ala Glu Glu Leu Ala Ser Leu Ala Ser
Val Lys Ala Ser Glu Leu Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO: 105

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Lys Leu Ser Pro Leu Leu Glu
Ser Phe Lys Val Ser Phe Leu Ser Ala Leu Glu Glu
Tyr Thr Lys Lys;

SEQ ID NO: 106

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Lys Leu Ser Pro Leu Lys Lys
Thr Tyr Glu Glu Leu Ala Ser Leu Phe Ser Val Lys
Phe Ser Glu Leu Ala Leu Ser Pro Leu Pro Arg Gly
Gly Ser Val Leu Val Thr;

SEQ ID NO: 107

Lys Lys Thr Ala Glu Glu Leu Ala Ser Leu Ala Ser
Val Lys Ala Ser Glu Leu Lys Leu Ser Pro Leu Lys
Lys Thr Tyr Glu Glu Leu Ala Ser Leu Phe Ser Val
Lys Phe Ser Glu Leu Ala Leu Ser Pro Leu Pro Arg
Gly Gly Ser Val Leu Val Thr;

SEQ ID NO: 108

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Lys Leu Ser Pro Leu Lys Lys
Thr Tyr Glu Glu Leu Ala Ser Leu Phe Ser Val Lys
Phe Ser Glu Leu;

SEQ ID NO: 109

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Pro Leu Glu Ser Phe Lys Val
Ser Phe Leu Ser Ala Leu Glu Glu Tyr Thr Lys Lys
Ala Leu Ser Pro Leu Pro Arg Gly Gly Ser Val Leu
Val Thr;

SEQ ID NO: 110

Leu Glu Ser Ala Lys Val Ser Ala Leu Ser Ala Leu
Glu Glu Ala Thr Lys Lys Pro Leu Glu Ser Phe Lys
Val Ser Phe Leu Ser Ala Leu Glu Glu Tyr Thr Lys
Lys Ala Leu Ser Pro Leu Pro Arg Gly Gly Ser Val
Leu Val Thr;

SEQ ID NO: 111

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Pro Leu Glu Ser Phe Lys Val
Ser Phe Leu Ser Ala Leu Glu Glu Tyr Thr Lys Lys;

SEQ ID NO: 112

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Leu Glu Ser Phe Lys Val
Ser Phe Leu Ser Ala Leu Glu Glu Tyr Thr Lys Lys
Ala Leu Ser Pro Leu Pro Arg Gly Gly Ser Val Leu
Val Thr;

SEQ ID NO: 113

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Ala Leu Ser Pro Leu Pro Arg Gly Gly Ser Val
Leu Val Thr;

SEQ ID NO: 114

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Leu Glu Ser Phe Lys Val
Ser Phe Leu Ser Ala Leu Glu Glu Tyr Thr Lys Lys;

SEQ ID NO: 115

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Lys Lys Thr Tyr Glu Glu
Leu Ala Ser Leu Phe Ser Val Lys Phe Ser Glu Leu
Ala Leu Ser Pro Leu Pro Arg Gly Gly Ser Val Leu
Val Thr;

SEQ ID NO: 116

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Ala Leu Ser Pro Leu Pro Arg Gly Gly Ser Val
Leu Val Thr;
and,

SEQ ID NO: 117

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Lys Lys Thr Tyr Glu Glu
Leu Ala Ser Leu Phe Ser Val Lys Phe Ser Glu Leu

In a further embodiment, peptides of the present invention are described by the following subgeneric formula IV, in which one or more additional elements indicated as variables G and H, are added to formula I to make subgeneric formula IV.

G-(A-B-C)ₙ-H IV

(A-B-C)ₙ are as described in formula I above,

G is absent or present and is a peptide as defined in the present specification. In one embodiment, G is SEQ ID NO: 9 Ser Pro Leu or a conservative substitution thereof. These amino acids may also appear in reverse orientation as in SEQ ID NO: 10 Leu Pro Ser. It is to be understood that one or more of the amino acids in the G peptide may be D amino acids.

H is absent or present and is a peptide as defined in the present specification. In one embodiment, H is SEQ ID NO: 23 Leu Asn Thr Gln or a conservative substitution thereof. These amino acids may also appear in reverse orientation as in SEQ ID NO: 24 Gln Thr Asn Leu. It is to be understood that one or more of the amino acids in the H peptide may be D amino acids.

Specific embodiments of peptides represented by generic formula IV are as follows:

### Wherein A is 5 or 6 and C is 8

SEQ ID NO: 118

Ser Pro Leu Gly Glu Glu Met Arg Asp Arg Ala Arg
Ala His Val Asp Ala Leu Arg Thr His Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO: 119

Gly Glu Glu Met Arg Asp Arg Ala Arg Ala His Val
Asp Ala Leu Arg Thr His Lys Leu Ser Pro Leu Leu
Glu Ser Phe Lys Val Ser Phe Leu Ser Ala Leu Glu
Glu Tyr Thr Lys Lys Leu Asn Thr Gln;

SEQ ID NO: 120

Ser Pro Leu Gly Glu Glu Met Arg Asp Arg Ala Arg
Ala His Val Asp Ala Leu Arg Thr His Lys Leu Ser
Pro Leu Leu Glu Ser Phe Lys Val Ser Phe Leu Ser
Ala Leu Glu Glu Tyr Thr Lys Lys;

SEQ ID NO: 121

Ser Pro Leu Ser Asp Glu Leu Arg Gln Arg Leu Ala
Ala Arg Leu Glu Ala Leu Lys Glu Asn Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO: 122

Ser Asp Glu Leu Arg Gln Arg Leu Ala Ala Arg Leu
Glu Ala Leu Lys Glu Asn Lys Leu Ser Pro Leu Leu
Glu Ser Phe Lys Val Ser Phe Leu Ser Ala Leu Glu
Glu Tyr Thr Lys Lys Leu Asn Thr Gln;

SEQ ID NO: 123

Ser Pro Leu Ser Asp Glu Leu Arg Gln Arg Leu Ala
Ala Arg Leu Glu Ala Leu Lys Glu Asn Lys Leu Ser
Pro Leu Leu Glu Ser Phe Lys Val Ser Phe Leu Ser
Ala Leu Glu Glu Tyr Thr Lys Lys;

SEQ ID NO: 124

Ser Pro Leu Gly Glu Glu Met Arg Asp Arg Ala Arg
Ala His Val Asp Ala Leu Arg Thr His Pro Leu Glu
Ser Phe Lys Val Ser Phe Leu Ser Ala Leu Glu Glu
Tyr Thr Lys Lys Leu Asn Thr Gln;

SEQ ID NO: 125

Gly Glu Glu Met Arg Asp Arg Ala Arg Ala His Val
Asp Ala Leu Arg Thr His Pro Leu Glu Ser Phe Lys
Val Ser Phe Leu Ser Ala Leu Glu Glu Tyr Thr Lys
Lys Leu Asn Thr Gln;

SEQ ID NO: 126

Ser Pro Leu Gly Glu Glu Met Arg Asp Arg Ala Arg
Ala His Val Asp Ala Leu Arg Thr His Pro Leu Glu
Ser Phe Lys Val Ser Phe Leu Ser Ala Leu Glu Glu
Tyr Thr Lys Lys;

SEQ ID NO: 127

Ser Pro Leu Ser Asp Glu Leu Arg Gln Arg Leu Ala
Ala Arg Leu Glu Ala Leu Lys Glu Asn Pro Leu Glu
Ser Phe Lys Val Ser Phe Leu Ser Ala Leu Glu Glu
Tyr Thr Lys Lys Leu Asn Thr Gln;

SEQ ID NO: 128

Ser Asp Glu Leu Arg Gln Arg Leu Ala Ala Arg Leu
Glu Ala Leu Lys Glu Asn Pro Leu Glu Ser Phe Lys
Val Ser Phe Leu Ser Ala Leu Glu Glu Tyr Thr Lys
Lys Leu Asn Thr Gln;
and,

SEQ ID NO: 129

Ser Pro Leu Ser Asp Glu Leu Arg Gln Arg Leu Ala
Ala Arg Leu Glu Ala Leu Lys Glu Asn Pro Leu Glu
Ser Phe Lys Val Ser Phe Leu Ser Ala Leu Glu Glu
Tyr Thr Lys Lys.

### Wherein A is 8′ and C is 8

SEQ ID NO: 130

Ser Pro Leu Leu Glu Ser Ala Lys Val Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO: 131

Leu Glu Ser Ala Lys Val Ser Ala Leu Ser Ala Leu
Glu Glu Ala Thr Lys Lys Lys Leu Ser Pro Leu Leu
Glu Ser Phe Lys Val Ser Phe Leu Ser Ala Leu Glu
Glu Tyr Thr Lys Lys Leu Asn Thr Gln;

SEQ ID NO: 132

Ser Pro Leu Leu Glu Ser Ala Lys Val Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys;

SEQ ID NO: 133

Ser Pro Leu Lys Lys Thr Ala Glu Glu Leu Ala Ser
Leu Ala Ser Val Lys Ala Ser Glu Leu Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO: 134

Lys Lys Thr Ala Glu Glu Leu Ala Ser Leu Ala Ser
Val Lys Ala Ser Glu Leu Lys Leu Ser Pro Leu Leu
Glu Ser Phe Lys Val Ser Phe Leu Ser Ala Leu Glu
Glu Tyr Thr Lys Lys Leu Asn Thr Gln;

SEQ ID NO: 135

Ser Pro Leu Lys Lys Thr Ala Glu Glu Leu Ala Ser
Leu Ala Ser Val Lys Ala Ser Glu Leu Lys Leu Ser
Pro Leu Leu Glu Ser Phe Lys Val Ser Phe Leu Ser
Ala Leu Glu Glu Tyr Thr Lys Lys;

SEQ ID NO: 136

Ser Pro Leu Lys Lys Thr Ala Glu Glu Leu Ala Ser
Leu Ala Ser Val Lys Ala Ser Glu Leu Lys Leu Ser
Pro Leu Lys Lys Thr Tyr Glu Glu Leu Ala Ser Leu
Phe Ser Val Lys Phe Ser Glu Leu Leu Asn Thr Gln;

SEQ ID NO: 137

Lys Lys Thr Ala Glu Glu Leu Ala Ser Leu Ala Ser
Val Lys Ala Ser Glu Leu Lys Leu Ser Pro Leu Lys
Lys Thr Tyr Glu Glu Leu Ala Ser Leu Phe Ser Val
Lys Phe Ser Glu Leu Leu Asn Thr Gln;

SEQ ID NO: 138

Ser Pro Leu Lys Lys Thr Ala Glu Glu Leu Ala Ser
Leu Ala Ser Val Lys Ala Ser Glu Leu Lys Leu Ser
Pro Leu Lys Lys Thr Tyr Glu Glu Leu Ala Ser Leu
Phe Ser Val Lys Phe Ser Glu Leu;

SEQ ID NO: 139

Ser Pro Leu Leu Glu Ser Ala Lys Val Ser Ala Leu
Ser Ala Leu Glu Glu Ala Thr Lys Lys Lys Leu Ser
Pro Leu Lys Lys Thr Tyr Glu Glu Leu Ala Ser Leu
Phe Ser Val Lys Phe Ser Glu Leu Leu Asn Thr Gln;

SEQ ID NO: 140

Leu Glu Ser Ala Lys Val Ser Ala Leu Ser Ala Leu
Glu Glu Ala Thr Lys Lys Lys Leu Ser Pro Leu Lys
Lys Thr Tyr Glu Glu Leu Ala Ser Leu Phe Ser Val
Lys Phe Ser Glu Leu Leu Asn Thr Gln;

SEQ ID NO: 141

Ser Pro Leu Leu Glu Ser Ala Lys Val Ser Ala Leu
Ser Ala Leu Glu Glu Ala Thr Lys Lys Lys Leu Ser
Pro Leu Lys Lys Thr Tyr Glu Glu Leu Ala Ser Leu
Phe Ser Val Lys Phe Ser Glu Leu;

SEQ ID NO: 142

Ser Pro Leu Leu Glu Ser Ala Lys Val Ser Ala Leu
Ser Ala Leu Glu Glu Ala Thr Lys Lys Pro Leu Glu
Ser Phe Lys Val Ser Phe Leu Ser Ala Leu Glu Glu
Tyr Thr Lys Lys Leu Asn Thr Gln;

SEQ ID NO: 143

Leu Glu Ser Ala Lys Val Ser Ala Leu Ser Ala Leu
Glu Glu Ala Thr Lys Lys Pro Leu Glu Ser Phe Lys
Val Ser Phe Leu Ser Ala Leu Glu Glu Tyr Thr Lys
Lys Leu Asn Thr Gln;

SEQ ID NO: 144

Ser Pro Leu Leu Glu Ser Ala Lys Val Ser Ala Leu
Ser Ala Leu Glu Glu Ala Thr Lys Lys Pro Leu Glu
Ser Phe Lys Val Ser Phe Leu Ser Ala Leu Glu Glu
Tyr Thr Lys Lys;

SEQ ID NO: 145

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys Leu Asn Thr Gln;

SEQ ID NO: 146

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Leu Asn Thr Gln;

SEQ ID NO: 147

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys;

SEQ ID NO: 148

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu Leu Asn Thr Gln;

SEQ ID NO: 149

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Leu Asn Thr Gln;

SEQ ID NO: 150

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu;

SEQ ID NO: 151

Ser Pro Leu Leu Glu Ser Ala Lys Val Ser Ala Leu
Ser Ala Leu Glu Glu Ala Thr Lys Lys Pro Lys Lys
Thr Tyr Glu Glu Leu Ala Ser Leu Phe Ser Val Lys
Phe Ser Glu Leu Leu Asn Thr Gln;

SEQ ID NO: 152

Leu Glu Ser Ala Lys Val Ser Ala Leu Ser Ala Leu
Glu Glu Ala Thr Lys Lys Pro Lys Lys Thr Tyr Glu
Glu Leu Ala Ser Leu Phe Ser Val Lys Phe Ser Glu
Leu Leu Asn Thr Gln;
and,

SEQ ID NO: 153

Ser Pro Leu Leu Glu Ser Ala Lys Val Ser Ala Leu
Ser Ala Leu Glu Glu Ala Thr Lys Lys Pro Lys Lys
Thr Tyr Glu Glu Leu Ala Ser Leu Phe Ser Val Lys
Phe Ser Glu Leu.

In a further embodiment, peptides of the present invention are described by the following subgeneric formula V, in which one or more additional elements indicated as variables D, E, F, W, G and H are added to formula I to make subgeneric formula V.

D-E-G-(A-B-C)ₙ-H-F-W V

(A-B-C)ₙ are as described in formula I above,

D is absent or present and is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

E is absent or present and is a group linking D and A and is Pro, SEQ ID NO: 10 Leu Pro Ser Leu Lys, or a conservative substitution thereof, provided that E is present only when D is present. These amino acids may also appear in reverse orientation as in SEQ ID NO: 7 Lys Leu Ser Pro Leu. When D is absent, E is absent.

F is absent or present and is a group linking C and W and is Pro, SEQ ID NO: 19 Ala Leu Ser Pro Leu, or a conservative substitution thereof, provided that F is present only when W is present. These amino acids may also appear in reverse orientation as in SEQ ID NO: 20 Leu Pro Ser Leu Ala. When W is absent, F is absent.

W is absent or present and is a peptide as defined in the present specification. In one embodiment, W is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

G is absent or present and is a peptide as defined in the present specification provided that G is present when D is absent. In one embodiment, G is SEQ ID NO: 9 Ser Pro Leu or a conservative substitution thereof. These amino acids may also appear in reverse orientation as in SEQ ID NO: 12 Leu Pro Ser. It is to be understood that one or more of the amino acids in the G peptide may be D amino acids.

H is absent or present and is a peptide as defined in the present specification provided that H is present when W is absent. In one embodiment, H is SEQ ID NO: 23 Leu Asn Thr Gln or a conservative substitution thereof. These amino acids may also appear in reverse orientation as in SEQ ID NO: 24 Gln Thr Asn Leu. It is to be understood that one or more of the amino acids in the H peptide may be D amino acids.

As stated above, in generic formula V, variables D or W may be absent or present. In one embodiment, D is present and W is absent. In another embodiment, W is present and D is absent. In another embodiment, both D and W are present.

Specific embodiments of peptides represented by generic formula V are:

### Wherein A is 5 or 6 and C is 8

SEQ ID NO: 154

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Lys Leu Ser Pro
Leu Leu Glu Ser Phe Lys Val Ser Phe Leu Ser Ala
Leu Glu Glu Tyr Thr Lys Lys Leu Asn Thr Gln;

SEQ ID NO: 155

Ser Pro Leu Gly Glu Glu Met Arg Asp Arg Ala Arg
Ala His Val Asp Ala Leu Arg Thr His Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO: 156

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Lys Leu Ser Pro
Leu Leu Glu Ser Phe Lys Val Ser Phe Leu Ser Ala
Leu Glu Glu Tyr Thr Lys Lys Leu Asn Thr Gln;

SEQ ID NO: 157

Ser Pro Leu Ser Asp Glu Leu Arg Gln Arg Leu Ala
Ala Arg Leu Glu Ala Leu Lys Glu Asn Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO: 158

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Pro Leu Glu Ser
Phe Lys Val Ser Phe Leu Ser Ala Leu Glu Glu Tyr
Thr Lys Lys Leu Asn Thr Gln;

SEQ ID NO: 159

Ser Pro Leu Gly Glu Glu Met Arg Asp Arg Ala Arg
Ala His Val Asp Ala Leu Arg Thr His Pro Leu Glu
Ser Phe Lys Val Ser Phe Leu Ser Ala Leu Glu Glu
Tyr Thr Lys Lys Ala Leu Ser Pro Leu Tm Arg Trp
Trp Trp Trp;

SEQ ID NO: 160

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Pro Leu Glu Ser
Phe Lys Val Ser Phe Leu Ser Ala Leu Glu Glu Tyr
Thr Lys Lys Leu Asn Thr Gln;
or,

SEQ ID NO: 161

Ser Pro Leu Ser Asp Glu Leu Arg Gln Arg Leu Ala
Ala Arg Leu Glu Ala Leu Lys Glu Asn Pro Leu Glu
Ser Phe Lys Val Ser Phe Leu Ser Ala Leu Glu Glu
Tyr Thr Lys Lys Ala Leu Ser Pro Leu Trp Arg Trp
Trp Trp Trp.

### Wherein A is 8′ and C is 8

SEQ ID NO: 162

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Lys Leu Ser Pro
Leu Leu Glu Ser Phe Lys Val Ser Phe Leu Ser Ala
Leu Glu Glu Tyr Thr Lys Lys Leu Asn Thr Gln;

SEQ ID NO: 163

Ser Pro Leu Leu Glu Ser Ala Lys Val Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO: 164

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Leu Glu Ser Phe Lys Val Ser Phe Leu Ser Ala
Leu Glu Glu Tyr Thr Lys Lys Leu Asn Thr Gln;

SEQ ID NO: 165

Ser Pro Leu Lys Lys Thr Ala Glu Glu Leu Ala Ser
Leu Ala Ser Val Lys Ala Ser Glu Leu Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO: 166

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Leu Ser Pro
Leu Lys Lys Thr Tyr Glu Glu Leu Ala Ser Leu Phe
Ser Val Lys Phe Ser Glu Leu Leu Asn Thr Gln;

SEQ ID NO: 167

Ser Pro Leu Lys Lys Thr Ala Glu Glu Leu Ala Ser
Leu Ala Ser Val Lys Ala Ser Glu Leu Lys Leu Ser
Pro Leu Lys Lys Thr Tyr Glu Glu Leu Ala Ser Leu
Phe Ser Val Lys Phe Ser Glu Leu Ala Leu Ser Pro
Leu Trp Arg Trp Trp Trp Trp;

SEQ ID NO: 168

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Lys Leu Ser Pro
Leu Lys Lys Thr Tyr Glu Glu Leu Ala Ser Leu Phe
Ser Val Lys Phe Ser Glu Leu Leu Asn Thr Gln;

SEQ ID NO: 169

Ser Pro Leu Leu Glu Ser Ala Lys Val Ser Ala Leu
Ser Ala Leu Glu Glu Ala Thr Lys Lys Lys Leu Ser
Pro Leu Lys Lys Thr Tyr Glu Glu Leu Ala Ser Leu
Phe Ser Val Lys Phe Ser Glu Leu Ala Leu Ser Pro
Leu Trp Arg Trp Trp Trp Trp;

SEQ ID NO: 170

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Pro Leu Glu Ser
Phe Lys Val Ser Phe Leu Ser Ala Leu Glu Glu Tyr
Thr Lys Lys Leu Asn Thr Gln;

SEQ ID NO: 171

Ser Pro Leu Leu Glu Ser Ala Lys Val Ser Ala Leu
Ser Ala Leu Glu Glu Ala Thr Lys Lys Pro Leu Glu
Ser Phe Lys Val Ser Phe Leu Ser Ala Leu Glu Glu
Tyr Thr Lys Lys Ala Leu Ser Pro Leu Trp Arg Trp
Trp Trp Trp;

SEQ ID NO: 172

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys Leu Asn Thr Gln;

SEQ ID NO: 173

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys Ala Leu Ser Pro Leu Trp Arg Trp
Trp Trp Trp;

SEQ ID NO: 174

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu Leu Asn Thr Gln;

SEQ ID NO: 175

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu Ala Leu Ser Pro Leu Trp Arg Trp
Trp Trp Trp;

SEQ ID NO: 176

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Pro Lys Lys Thr
Tyr Glu Glu Leu Ala Ser Leu Phe Ser Val Lys Phe
Ser Glu Leu Leu Asn Thr Gln;
and

SEQ ID NO: 177

Ser Pro Leu Leu Glu Ser Ala Lys Val Ser Ala Leu
Ser Ala Leu Glu Glu Ala Thr Lys Lys Pro Lys Lys
Thr Tyr Glu Glu Leu Ala Ser Leu Phe Ser Val Lys
Phe Ser Glu Leu Ala Leu Ser Pro Leu Trp Arg Trp
Trp Trp Trp

In a further embodiment, peptides of the present invention are described by formula VI,

D-I-W VI

D is a peptide as defined in the present specification. In one embodiment, D is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D, namely SEQ ID NO: 17 Pro Arg Gly Gly Ser Val or SEQ ID NO: 18 Thr Val Leu Val Ser Gly may occur as D-amino acids;

I is a group linking D and W and is GABA, Pro, SEQ ID NO: 7 Lys Leu Ser Pro Leu, SEQ ID NO: 25 Leu Lys Leu Ser Pro Leu, or a conservative substitution thereof, or multiples thereof or combinations thereof. These amino acids may also appear in reverse orientation, for example, SEQ ID NO: 26 Leu Pro Ser Leu Lys Leu.

W is absent or present and is a peptide as defined in the present specification. In one embodiment, W is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W peptide may be D amino acids.

D and W may also be switched in location in D-I-W to form W-I-D.

Specific embodiments of peptides represented by formula VI are:

SEQ ID NO: 178

Pro Arg Gly Gly Ser Val Leu Val Thr Leu Lys Leu
Ser Pro Leu Trp Arg Trp Trp Trp Trp;

SEQ ID NO: 179

Trp Arg Trp Trp Trp Trp Leu Lys Leu Ser Pro Leu
Pro Arg Gly Gly Ser Val Leu Val Thr;

SEQ ID NO: 180

Pro Arg Gly Gly Ser Val Leu Val Thr -(GABA)-
Pro Trp Arg Trp Trp Trp Trp;

SEQ ID NO: 181

Pro Arg Gly Gly Ser Val Leu Val Thr -(GABA-GABA)
Pro Trp Arg Trp Trp Trp Trp;

SEQ ID NO: 182

Pro Arg Gly Gly Ser Val Leu Val Thr -(GABA-GABA-
GABA)-Pro Trp Arg Trp Trp Trp Trp;

SEQ ID NO: 183

Pro Arg Gly Gly Ser Val Leu Val Thr -(GABA-GABA-
GABA-GABA) Pro Trp Arg Trp Trp Trp Trp;

SEQ ID NO: 184

Pro Arg Gly Gly Ser Val Leu Val Thr Pro Trp Arg
Trp Trp Trp Trp;

SEQ ID NO: 185

PRO Arg Gly Gly Ser Val Leu Val Thr -(GABA)- Pro
Trp Arg Trp Trp Trp Trp;

SEQ ID NO: 186

Pro ARG Gly Gly Ser Val Leu Val Thr -(GABA)- Pro
Trp Arg Trp Trp Trp Trp;

SEQ ID NO: 187

Pro Arg GLY Gly Ser Val Leu Val Thr -(GABA)- Pro
Trp Arg Trp Trp Trp Trp;

SEQ ID NO: 188

Pro Arg Gly GLY Ser Val Leu Val Thr -(GABA)-Pro
Trp Arg Trp Trp Trp Trp;

SEQ ID NO: 189

Pro Arg Gly Gly SER Val Leu Val Thr -(GABA)- Pro
Trp Arg Trp Trp Trp Trp;

SEQ ID NO: 190

Pro Arg Gly Gly Ser VAL Leu Val Thr -(GABA)- Pro
Trp Arg Trp Trp Trp Trp;

SEQ ID NO: 191

PRO Arg Gly Gly Ser Val Leu Val Thr Pro Trp Arg
Trp Trp Trp Trp;

SEQ ID NO: 192

Pro ARG Gly Gly Ser Val Leu Val Thr Pro Trp Arg
Trp Trp Trp Trp;

SEQ ID NO: 193

Pro Arg GLY Gly Ser Val Leu Val Thr Pro Trp Arg
Trp Trp Trp Trp;

SEQ ID NO: 194

Pro Arg Gly GLY Ser Val Leu Val Thr Pro Trp Arg
Trp Trp Trp Trp;

SEQ ID NO: 195

Pro Arg Gly Gly SER Val Leu Val Thr Pro Trp Arg
Trp Trp Trp Trp;

SEQ ID NO: 196

Pro Arg Gly Gly Ser VAL Leu Val Thr Pro Trp Arg
Trp Trp Trp Trp;

SEQ ID NO: 197

PRO Arg Gly Gly Ser Val Leu Val Thr -(GABA)- Pro
Trp Arg Trp Trp Trp TRP;

SEQ ID NO: 198

Pro Arg Gly Gly Ser Val Leu Val Thr Pro Trp Trp
Trp Trp Arg Trp;

SEQ ID NO: 199

PRO ARG GLY GLY SER VAL LEU VAL THR PRO TRP TRP
TRP TRP ARG TRP;

SEQ ID NO: 200

PRO Arg Gly Gly Ser Val Leu Val Thr Pro Trp Trp
Trp Trp Arg TRP;

SEQ ID NO: 201

Pro Arg Gly Gly Ser Val Leu Val Thr -(GABA)- Pro
Trp Trp Trp Trp Arg Trp;

SEQ ID NO: 202

PRO Arg Gly Gly Ser Val Leu Val Thr -(GABA)- Pro
Trp Trp Trp Trp Arg TRP;
and

SEQ ID NO: 203

PRO ARG GLY GLY SER VAL LEU VAL THR -(gaba)- PRO
TRP TRP TRP TRP ARG TRP.

In another embodiment, the peptides of the present invention are described by the following generic formula VII:

(D-R-S-W-T-N-O-(Xₙ-Y_{z}-Zₘ)ₛ-O′-N′-T′-W′-S′-R′-D′)ᵣ VII

wherein: n is an integer from 1 to 3, m is an integer from 1 to 3, r is an integer from 1 to 10, z is an integer from 1 to 13, and s is an integer from 1 to 5. It is to be understood that the letters in the generic formulae VII-XVIII or in components thereof are defined by the text that follows each letter and do not designate an individual amino acid.

D or D′ is individually absent or present and is a peptide as defined in the present specification. In one embodiment, D or D′ is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D or D′, namely SEQ ID NO: 17 Pro Arg Gly Gly Ser Val or SEQ ID NO: 18 Thr Val Leu Val Ser Gly may occur as D-amino acids;

R or R′ is individually absent or present and is a linking group comprised of at least one gamma aminobutyric acid (GABA), or one or more neutral amino acids. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for R or R′ include SEQ ID NO: 27 (Gly-Pro-Gly-Gly), and SEQ ID NO: 28 (Gly₄-Ser)_{y}, wherein x is an integer from about 1 to about 9 and y is an integer from about 1 to about 8. Suitable linking groups for R or R′ may be selected from the following without limitation:

GABA; GABA-GABA; GABA-GABA-GABA;

SEQ ID NO: 29

Gly Pro Gly Gly;

SEQ ID NO: 30

Gly Pro Gly Gly Gly Pro Gly Gly;

SEQ ID NO: 31

Gly Pro Gly Gly Gly Pro Gly Gly Gly Pro Gly Gly;

SEQ ID NO: 32

Gly Pro Gly Gly Gly Pro Gly Gly Gly Pro Gly Gly
Gly Pro Gly Gly;

SEQ ID NO: 33

Gly Gly Gly Gly Ser;

SEQ ID NO: 34

Gly Gly Gly Gly Ser Gly Gly Gly Gly Ser
or

SEQ ID NO: 35

Gly Gly Gly Gly Ser Gly Gly Gly Gly Ser Gly Gly
Gly Gly Ser;

S or S′ is individually absent or present and is a linking group comprised of amino acid residues of from 1 to 10 residues in length, wherein the amino acid residues are proline, alanine, leucine, lysine, serine, glycine, polymers thereof or combinations (co-polymers) thereof, or is comprised of an alkyl group (CH₂)ₙ, wherein n is an integer from 1-20;

W or W′ is individually absent or present and is a peptide as defined in the present specification. In one embodiment, W or W′ is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W or W′ peptide may be D amino acids;

T or T′ is individually absent or present and is a linking group comprised of at least one gamma aminobutyric acid (GABA), or one or more neutral amino acids. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for T or T′ include SEQ ID NO: 27 (Gly-Pro-Gly-Gly)ₓ and SEQ ID NO: 28 (Gly₄-Ser)_{y}, wherein x is an integer from about 1 to about 9 and y is an integer from about 1 to about 8. Suitable linking groups for T or T′ may be selected from the following without limitation: GABA; GABA-GABA; GABA-GABA-GABA; SEQ ID NO: 29 Gly Pro Gly Gly; SEQ ID NO: 30 Gly Pro Gly Gly Gly Pro Gly Gly; SEQ ID NO: 31 Gly Pro Gly Gly Gly Pro Gly Gly Gly Pro Gly Gly; SEQ ID NO: 32 Gly Pro Gly Gly Gly Pro Gly Gly Gly Pro Gly Gly Gly Pro Gly Gly; SEQ ID NO: 33 Gly Gly Gly Gly Ser; SEQ ID NO: 34 Gly Gly Gly Gly Ser Gly Gly Gly Gly Ser or SEQ ID NO: 35 Gly Gly Gly Gly Ser Gly Gly Gly Gly Ser Gly Gly Gly Gly Ser;

N or N′ is individually absent or present and is a linking group comprised of amino acid residues of from 1 to 10 residues in length, wherein the amino acid residues are proline, alanine, leucine, serine, glycine, polymers thereof or combinations (co-polymers) thereof, or is comprised of an alkyl group (CH₂)ₙ, wherein n is an integer from 1-20;

O or O′ is individually absent or present and is a linking group comprised of at least one GABA, or one or more neutral amino acids, or combinations thereof. Neutral amino acids that may be employed include, but are not limited to, proline, serine, leucine, alanine, valine, polymers thereof and combinations (co-polymers) thereof. For example, poly leucine, poly alanine, poly proline, poly valine, and poly serine may be used. Other substituents for O or O′ include SEQ ID NO: 27 (Gly-Pro-Gly-Gly)ₓ and SEQ ID NO: 28 (Gly₄-Ser)_{y}, wherein x is an integer from about 1 to about 9 and y is an integer from about 1 to about 8. Suitable linking groups for O or O′ may be selected from the following without limitation: GABA; GABA-GABA; GABA-GABA-GABA; SEQ ID NO: 29 Gly Pro Gly Gly; SEQ ID NO: 30 Gly Pro Gly Gly Gly Pro Gly Gly; SEQ ID NO: 31 Gly Pro Gly Gly Gly Pro Gly Gly Gly Pro Gly Gly; SEQ ID NO: 32 Gly Pro Gly Gly Gly Pro Gly Gly Gly Pro Gly Gly Gly Pro Gly Gly; SEQ ID NO: 33 Gly Gly Gly Gly Ser; SEQ ID NO: 34 Gly Gly Gly Gly Ser Gly Gly Gly Gly Ser or SEQ ID NO: 35 Gly Gly Gly Gly Ser Gly Gly Gly Gly Ser Gly Gly Gly Gly Ser;

X is a peptide comprised of from 5 to 25 amino acid residues, provided an amphipathic alpha helix is obtained. Examples of X are provided below in the specification;

Y is absent or present and is a linking group comprised of amino acid(s) of from 1 to 10 residues in length, wherein the amino acid residues are proline, alanine, leucine, serine, glycine, lysine, polymers thereof or combinations (co-polymers) thereof, or is comprised of an alkyl group (CH₂)ₙ, wherein n is an integer from 1-20; z is an integer from 1 to 13 and refers to the number of times Y may be present in Xₙ-Y_{z}-Zₘ when n or m in Xₙ-Y_{z}-Zₘ are more than 1 or when s is more than 1;

Z is a peptide comprised of from 5 to 25 amino acids residues, provided an amphipathic alpha helix is obtained. Examples of Z are provided below in the specification, provided that more than one 1 amphipathic alpha helical domain is present when X and Z are taken in combination.

The present invention also provides peptides of the following subgeneric formulae VIII and IX, wherein the variables are as described in formula VII:

(D-R-S-W-T-N-O-(Xₙ-Y_{z}-Zₘ)ₛ)ᵣ VIII

((Xₙ-Y_{z}-Zₘ)ₛ-O′-N′-T′-W′-S′-R′-D′)ᵣ IX

In some embodiments, the D may be placed in the position of W and W may be placed in the position of D in formula VII to yield the following:

(W-R-S-D-T-N-O-(Xₙ-Y_{z}-Zₘ)ₛ-O′-N′-T′-W′-S′-R′-D′)ᵣ X

In some embodiments, the D′ may be placed in the position of W′, and W′ may be placed in the position of D′ in formula VII to yield the following:

(D-R-S-W-T-N-O-(Xₙ-Y_{z}-Zₘ)ₛ-O′-N′-T′-D′-S′-R′-W′)ᵣ XI

In some embodiments, the D may be placed in the position of W and W may be placed in the position of D in formula VII and D′ may be placed in the position of W′, and W′ may be placed in the position of D′ in formula VII to yield the following:

(W-R-S-D-T-N-O-(Xₙ-Y_{z}-Zₘ)ₛ-O′-N′-T′-D′-S′-R′W′)ᵣ XII

The present invention also provides peptides of the following formulae:

(W-R-S-D-T-N-O-(Xₙ-Y_{z}-Zₘ)ₛ)ᵣ XIII

((Xₙ-Y_{z}-Zₘ)ₛ-O′-N′-T′-D′-S′-R′-W′)ᵣ XIV

(D-R-S-T-N-O-(Xₙ-Y_{z}-Zₘ)ₛ)ᵣ XV

D-W XVI

W-D XVII

(Xₙ-Y_{z}-Zₘ)ₛ XVIII

The present invention also includes compositions comprising combinations of individual peptides of the present invention in an acceptable carrier. For example, a mixture of D, W, and (Xₙ-Y_{z}-Zₘ)ₛ may be made in an acceptable carrier. These peptides are as defined above and may be labeled or unlabelled. It is to be understood that a mixture of peptides, such as D, W, and (Xₙ-Y_{z}-Zₘ)ₛ may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity.

### The Xₙ-Y_{z}-Zₘ Component of Formulae VII-XV and XVIII

The Xₙ-Y_{z}-Zₘ component of formula VII-XV and XVIII require that more than one 1 amphipathic alpha helical domain is present when X and Z are taken in combination.

While not wanting to be bound by the following statement, it is believed that the Xₙ-Y_{z}-Zₘ component of formula VII, or formulae VIII-XV or XVIII alone or in combination with the other components of formula VII, or formulae VIII-XV or XVIII facilitate lipid efflux from cells. While not wanting to be bound by the following statement, it is believed that the Xₙ-Y_{z}-Zₘ component of formula VII, or formulae VIII-XV or XVIII, alone or in combination with the other components of formula VII facilitate lipid efflux from cells through an ABCAl dependent pathway.

In one embodiment of Xₙ-Y_{z}-Zₘ, n=l, m=1, and z=1, Y is proline, X is SEQ ID NO: 204 Asp Trp Leu Lys Ala Phe Tyr Asp Lys Val Ala Glu Lys Leu Lys Glu Ala Phe, and Z is SEQ ID NO: 205 Asp Trp Ala Lys Ala Ala Tyr Asp Lys Ala Ala Glu Lys Ala Lys Glu Ala Ala, to yield the following peptide, SEQ ID NO: 206 Asp Trp Leu Lys Ala Phe Tyr Asp Lys Val Ala Glu Lys Leu Lys Glu Ala Phe Pro Asp Trp Ala Lys Ala Ala Tyr Asp Lys Ala Ala Glu Lys Ala Lys Glu Ala Ala

It is to be understood that X and Z may be the same or different. In one embodiment, X and Z may be the same or different and may be SEQ ID NO: 204 Asp Trp Leu Lys Ala Phe Tyr Asp Lys Val Ala Glu Lys Leu Lys Glu Ala Phe or SEQ ID NO: 205 Asp Trp Ala Lys Ala Ala Tyr Asp Lys Ala Ala Glu Lys Ala Lys Glu Ala Ala. Y may be Proline (P) and is often shown as -P- in the sequences that follow. Any one or more of the C-terminal six amino acids of Z, specifically SEQ ID NO: 207 Lys Ala Lys Glu Ala Ala may be D amino acids. It is further to be understood that Xₙ-Y_{z}-Zₘ, and the embodiment

SEQ ID NO: 206

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala

includes the following variations without limitation:
a) changes in length (N terminal and or C terminal deletions of X and or Z);
b) multiple repeats;
c) change in orientation of the X peptide relative to Z peptide in the overall Xₙ-Y_{z}-Zₘ peptide;
d) reversal of the N terminal to C terminal orientation of the X peptide;
e) reversal of the N terminal to C terminal orientation of the Z peptide;
f) reversal of the N terminal to C terminal orientation of the X peptide, and
reversal of the N terminal to C terminal orientation of the Z peptide;
g) reversal of the N terminal to C terminal orientation of Z and reversal of the N terminal to C terminal orientation of X and change in the order, Z-Y-X;
h) reversal of the N terminal to C terminal orientation of Z when Z is SEQ ID NO: 204 Asp Trp Leu Lys Ala Phe Tyr Asp Lys Val Ala Glu Lys Leu Lys Glu Ala Phe, and X is SEQ ID NO: 205 Asp Trp Ala Lys Ala Ala Tyr Asp Lys Ala Ala Glu Lys Ala Lys Glu Ala Ala, in the order X-Y-Z;
i) insertion of D amino acids in the C-terminal six amino acids of Z, specifically SEQ ID NO: 207 Lys Ala Lys Glu Ala Ala; and,
j) insertion of a D amino acid at the N terminal of X or Z and/or at the C-terminal of X or Z.
The following are specific embodiments of these variations:
a) changes in length (N terminal and or C terminal deletions of X and or Z),

SEQ ID NO: 206

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala

1. Deletion of Residues from the C Terminal of X with Z Intact:

SEQ ID NO: 208

Asp Trp Leu Lys Ala Phe Tyr Asp Lys val Ala Glu
Lys Leu Lys Glu Ala Pro Asp Trp Ala Lys Ala Ala
Tyr Asp Lys Ala Ala Glu Lys Ala Lys Glu Ala Ala;

SEQ ID NO: 209

Asp Trp Leu Lys Ala Phe Tyr Asp Lys Val Ala Glu
Lys Leu Lys Glu Pro Asp Trp Ala Lys Ala Ala Tyr
Asp Lys Ala Ala Glu Lys Ala Lys Glu Ala Ala;

SEQ ID NO: 210

Asp Trp Leu Lys Ala Phe Tyr Asp Lys Val Ala Glu
Lys Leu Lys Pro Asp Trp Ala Lys Ala Ala Tyr Asp
Lys Ala Ala Glu Lys Ala Lys Glu Ala Ala;

SEQ ID NO: 211

Asp Trp Leu Lys Ala Phe Tyr Asp Lys Val Ala Glu
Lys Leu Pro Asp Trp Ala Lys Ala Ala Tyr Asp Lys
Ala Ala Glu Lys Ala Glu Ala Ala;

SEQ ID NO: 212

Asp Trp Leu Lys Ala Phe Tyr Asp Lys Val Ala Glu
Lys Pro Asp Trp Ala Lys Ala Ala Tyr Asp Lys Ala
Ala Glu Lys Ala Lys Glu Ala Ala;

SEQ ID NO: 213

Asp Trp Leu Lys Ala Phe Tyr Asp Lys Val Ala Glu
Pro Asp Trp Ala Lys Ala Ala Tyr Asp Lys Ala Ala
Glu Lys Ala Lys Glu Ala Ala;

2. Deletion of Residues from the C Terminal of Z with X Intact:

SEQ ID NO: 214

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala;

SEQ ID NO: 215

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu;

SEQ ID NO: 216

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys;

SEQ ID NO: 217

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala;

SEQ ID NO: 218

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys;

SEQ ID NO: 219

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu;

SEQ ID NO: 220

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala;

3. Deletion of Residues from the N Terminal of X with Z Intact:

SEQ ID NO: 221

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala;

SEQ ID NO: 222

Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala;

SEQ ID NO: 223

Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala;

SEQ ID NO: 224

Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala Ala;

SEQ ID NO: 225

Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 226

Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala;

4. Deletion of Residues from the N Terminal of Z with X Intact:

SEQ ID NO: 227

Asp Trp Leu Lys Ala Phe Tyr Asp Lys Val Ala Glu
Lys Leu Lys Glu Ala Phe Pro Trp Ala Lys Ala Ala
Tyr Asp Lys Ala Ala Glu Lys Ala Lys Glu Ala Ala;

SEQ ID NO: 228

Asp Trp Leu Lys Ala Phe Tyr Asp Lys Val Ala Glu
Lys Leu Lys Glu Ala Phe Pro Ala Lys Ala Ala Tyr
Asp Lys Ala Ala Glu Lys Ala Lys Glu Ala Ala;

SEQ ID NO: 229

Asp Trp Leu Lys Ala Phe Tyr Asp Lys Val Ala Glu
Lys Leu Lys Glu Ala Phe Pro Lys Ala Ala Tyr Asp
Lys Ala Ala Glu Lys Ala Lys Glu Ala Ala;

SEQ ID NO: 230

Asp Trp Leu Lys Ala Phe Tyr Asp Lys Val Ala Glu
Lys Leu Lys Glu Ala Phe Pro Ala Ala Tyr Asp Lys
Ala Ala Glu Lys Ala Lys Glu Ala Ala;

SEQ ID NO: 231

Asp Trp Leu Lys Ala Phe Tyr Asp Lys Val Ala Glu
Lys Leu Lys Glu Ala Phe Pro Ala Tyr Asp Lys Ala
Ala Glu Lys Ala Lys Glu Ala Ala;

SEQ ID NO: 232

Asp Trp Leu Lys Ala Phe Tyr Asp Lys Val Ala Glu
Lys Leu Lys Glu Ala Phe Pro Tyr Asp Lys Ala Ala
Glu Lys Ala Lys Glu Ala Ala;

5. Deletion of Residues from the N Terminal of X and the N Terminal of Z:

SEQ ID NO: 233

Trp Leu Lys Ala Phe Tyr Asp Lys Val Ala Glu Lys
Leu Lys Glu Ala Phe Pro Trp Ala Lys Ala Ala Tyr
Asp Lys Ala Ala Glu Lys Ala Lys Glu Ala Ala;

SEQ ID NO: 234

Leu Lys Ala Phe Tyr Asp Lys Val Ala Glu Lys Leu
Lys Glu Ala Phe Pro Ala Lys Ala Ala Tyr Asp Lys
Ala Ala Glu Lys Ala Lys Glu Ala Ala;

SEQ ID NO: 235

Lys Ala Phe Tyr Asp Lys Val Ala Glu Lys Leu Lys
Glu Ala Phe Pro Lys Ala Ala Tyr Asp Lys Ala Ala
Glu Lys Ala Lys Glu Ala Ala;

SEQ ID NO: 236

Ala Phe Tyr Asp Lys Val Ala Glu Lys Leu Lys Glu
Ala Phe Pro Ala Ala Tyr Asp Lys Ala Ala Glu Lys
Ala Lys Glu Ala Ala;

SEQ ID NO: 237

Phe Tyr Asp Lys Val Ala Glu Lys Leu Lys Glu Ala
Phe Pro Ala Tyr Asp Lys Ala Ala Glu Lys Ala Lys
Glu Ala Ala;

SEQ ID NO: 238

Tyr Asp Lys Val Ala Glu Lys Leu Lys Glu Ala Phe
Pro Tyr Asp Lys Ala Ala Glu Lys Ala Lys Glu Ala
Ala;

6. Deletion of Residues from the C Terminal of X and the C Terminal of Z:

SEQ ID NO: 239

Asp Trp Leu Lys Ala Phe Tyr Asp Lys Val Ala Glu
Lys Leu Lys Glu Ala Pro Asp Trp Ala Lys Ala Ala
Tyr Asp Lys Ala Ala Glu Lys Ala Lys Glu Ala;

SEQ ID NO: 240

Asp Trp Leu Lys Ala Phe Tyr Asp Lys Val Ala Glu
Lys Leu Lys Glu Pro Asp Trp Ala Lys Ala Ala Tyr
Asp Lys Ala Ala Glu Lys Ala Lys Glu;

SEQ ID NO: 241

Asp Trp Leu Lys Ala Phe Tyr Asp Lys Val Ala Glu
Lys Leu Pro Asp Asp Trp Ala Lys Ala Ala Tyr Asp
Lys Ala Ala Glu Lys Ala Lys;

SEQ ID NO: 242

Asp Trp Leu Lys Ala Phe Tyr Asp Lys Val Ala Glu
Lys Leu Pro Asp Trp Ala Lys Ala Ala Tyr Asp Lys
Ala Ala Glu Lys Ala;

SEQ ID NO: 243

Asp Trp Leu Lys Ala Phe Tyr Asp Lys Val Ala Glu
Lys Pro Asp Trp Ala Lys Ala Ala Tyr Asp Lys Ala
Ala Glu;

SEQ ID NO: 244

Asp Trp Leu Lys Ala Phe Tyr Asp Lys Val Ala Glu
Pro Asp Trp Ala Lys Ala Ala Tyr Asp Lys Ala Ala
Glu;

SEQ ID NO: 245

Asp Trp Leu Lys Ala Phe Tyr Asp Lys Val Ala Pro
Asp Trp Ala Lys Ala Ala Tyr Asp Lys Ala Ala;

SEQ ID NO: 246

Asp Trp Leu Lys Ala Phe Tyr Asp Lys Val Pro Asp
Trp Ala Lys Ala Ala Tyr Asp Lys Ala;

7. Deletion of Residues from the C Terminal of X and the N Terminal of Z:

SEQ ID NO: 247

Asp Trp Leu Lys Ala Phe Tyr Asp Lys Val Ala Glu
Lys Leu Lys Glu Ala Pro Trp Ala Lys Ala Ala Tyr
Ala Ala Glu Lys Ala Lys Glu Ala Ala;

SEQ ID NO: 248

Asp Trp Leu Lys Ala Phe Tyr Asp Lys Val Ala Glu
Lys Leu Lys Glu Pro Ala Lys Ala Ala Tyr Asp Lys
Ala Ala Glu Lys Ala Lys Glu Ala Ala;

SEQ ID NO: 249

Asp Trp Leu Lys Ala Phe Tyr Asp Lys Val Ala Glu
Lys Leu Lys Pro Lys Ala Ala Tyr Asp Lys Ala Ala
Glu Lys Ala Lys Glu Ala Ala;

SEQ ID NO: 250

Asp Trp Leu Lys Ala Phe Tyr Asp Lys Val Ala Glu
Lys Leu Pro Ala Ala Tyr Asp Lys Ala Ala Glu Lys
Ala Lys Glu Ala Ala;

SEQ ID NO: 251

Asp Trp Leu Lys Ala Phe Tyr Asp Lys Val Ala Glu
Lys Pro Ala Tyr Asp Lys Ala Ala Glu Lys Ala Lys
Glu Ala Ala;

SEQ ID NO: 252

Asp Trp Leu Lys Ala Phe Tyr Asp Lys Val Ala Glu
Pro Tyr Asp Lys Ala Ala Glu Lys Ala Lys Glu Ala
Ala;

SEQ ID NO: 253

Asp Trp Leu Lys Ala Phe Tyr Asp Lys Val Ala Pro
Asp Lys Ala Ala Glu Lys Ala Lys Glu Ala Ala;

8. Deletion of Residues from the C Terminal of Z and the N Terminal of X:

SEQ ID NO: 254

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala;

SEQ ID NO: 255

Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu;

SEQ ID NO: 256

Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu;

SEQ ID NO: 257

Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala;

SEQ ID NO: 258

Phe Tyr Asp Lys Val Ala Glu Lys Leu Lys Glu Ala
Phe Pro Asp Trp Ala Lys Ala Ala Tyr Asp Lys Ala
Ala Glu Lys;

SEQ ID NO: 259

Tyr Asp Lys Val Ala Glu Lys Leu Lys Glu Ala Phe
Pro Asp Trp Ala Lys Ala Ala Tyr Asp Lys Ala Ala
Glu;

### b) Multiple Repeats

(Xₙ-Y_{z}-Zₘ)ₛ

wherein: n is an integer from 1 to 3, m is an integer from 1 to 3, z is an integer from 1 to 13, and s is an integer from 1 to 5, wherein in some embodiment when s is more than 1, Y is optionally present between repeating units of X-Y-Z, and in some embodiments when n or m is greater than 1, Y is optionally present between repeats of X and/or between repeats of Z and Y or Pro (P).

SEQ ID NO: 260

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala Phe Pro Asp Trp Ala
Lys Ala Ala Tyr Asp Lys Ala Ala Glu Lys Ala Lys
Glu Ala Ala;

SEQ ID NO: 261

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala Phe Pro Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Pro Asp
Trp Ala Lys Ala Ala Tyr Asp Lys Ala Ala Glu Lys
Ala Lys Glu Ala Ala;

SEQ ID NO: 262

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala Phe Pro Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Pro Asp
Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala;

SEQ ID NO: 263

Asp Trp Leu Lys Ala Phe Tyr Asp Lys Val Ala Glu
Lys Leu Lys Glu Ala Phe Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 264

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Asp Trp Ala Lys Ala Ala Tyr Asp Lys Ala
Ala Glu Lys Ala Lys Glu Ala Ala;

c) change in orientation of the X peptide relative to Z peptide in the Xₙ-Y_{z}-Zₘ peptide. Another embodiment of the Xₙ-Y_{z}-Zₘ peptide wherein X is SEQ ID NO: 205 Asp Trp Ala Lys Ala Ala Tyr Asp Lys Ala Ala Glu Lys Ala Lys Glu Ala Ala or a variant thereof and Z is SEQ ID NO: 204 Asp Trp Leu Lys Ala Phe Tyr Asp Lys Val Ala Glu Lys Leu Lys Glu Ala Phe or a variant thereof, and Y is proline (P), is

SEQ ID NO: 265

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe

1) Removal of Residues from the C Terminal of Z with X Intact:

SEQ ID NO: 266

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala;

SEQ ID NO: 267

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu;

SEQ ID NO: 268

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys;

SEQ ID NO: 269

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu;

SEQ ID NO: 270

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys;

SEQ ID NO: 271

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu;

2) Removal of Residues from the C Terminal of X with Z Intact:

SEQ ID NO: 272

Asp Trp Leu Lys Ala Phe Tyr Asp Lys Val Ala Glu
Lys Leu Lys Glu Ala Pro Asp Trp Leu Lys Ala Phe
Tyr Asp Lys Val Ala Glu Lys Leu Lys Glu Ala Phe;

SEQ ID NO: 273

Asp Trp Leu Lys Ala Phe Tyr Asp Lys Val Ala Glu
Lys Leu Lys Glu Pro Asp Trp Leu Lys Ala Phe Tyr
Asp Lys Val Ala Glu Lys Leu Lys Glu Ala Phe;

SEQ ID NO: 274

Asp Trp Leu Lys Ala Phe Tyr Asp Lys Val Ala Glu
Lys Leu Lys Pro Asp Trp Leu Lys Ala Phe Tyr Asp
Lys Val Ala Glu Lys Leu Lys Glu Ala Phe;

SEQ ID NO: 275

Asp Trp Leu Lys Ala Phe Tyr Asp Lys Val Ala Glu
Lys Leu Pro Asp Trp Leu Lys Ala Phe Tyr Asp Lys
Val Ala Glu Lys Leu Lys Glu Ala Phe;

SEQ ID NO: 276

Asp Trp Leu Lys Ala Phe Tyr Asp Lys Val Ala Glu
Lys Pro Asp Trp Leu Lys Ala Phe Tyr Asp Lys Val
Ala Glu Lys Leu Lys Glu Ala Phe;

SEQ ID NO: 277

Asp Trp Leu Lys Ala Phe Tyr Asp Lys Val Ala Glu
Pro Asp Trp Leu Lys Ala Phe Tyr Asp Lys Val Ala
Glu Lys Leu Lys Glu Ala Phe;

SEQ ID NO: 278

Asp Trp Leu Lys Ala Phe Tyr Asp Lys Val Ala Pro
Asp Trp Leu Lys Ala Phe Tyr Asp Lys Val Ala Glu
Lys Leu Lys Glu Ala Phe;

3) Removal of Residues from the N Terminal of Z with X Intact:

SEQ ID NO: 279

Asp Trp Ala Lys Ala Ala Tyr Asp Lys Ala Ala Glu
Lys Ala Lys Glu Ala Ala Pro Trp Leu Lys Ala Phe
Tyr Asp Lys Val Ala Glu Lys Leu Lys Glu Ala Phe;

SEQ ID NO: 280

Asp Trp Ala Lys Ala Ala Tyr Asp Lys Ala Ala Glu
Lys Ala Lys Glu Ala Ala Pro Leu Lys Ala Phe Tyr
Asp Lys Val Ala Glu Lys Leu Lys Glu Ala Phe;

SEQ ID NO: 281

Asp Trp Ala Lys Ala Ala Tyr Asp Lys Ala Ala Glu
Lys Ala Lys Glu Ala Ala Pro Lys Ala Phe Tyr Asp
Lys Val Ala Glu Lys Leu Lys Glu Ala Phe;

SEQ ID NO: 282

Asp Trp Ala Lys Ala Ala Tyr Asp Lys Ala Ala Glu
Lys Ala Lys Glu Ala Ala Pro Ala Phe Tyr Asp Lys
Val Ala Glu Lys Leu Lys Glu Ala Phe;

SEQ ID NO: 283

Asp Trp Ala Lys Ala Ala Tyr Asp Lys Ala Ala Glu
Lys Ala Lys Glu Ala Ala Pro Phe Tyr Asp Lys Val
Ala Glu Lys Leu Lys Glu Ala Phe;

SEQ ID NO: 284

Asp Trp Ala Lys Ala Ala Tyr Asp Lys Ala Ala Glu
Lys Ala Lys Glu Ala Ala Pro Asp Lys Val Ala Glu
Lys Leu Lys Glu Ala Phe;

4) Removal of Residues from the N Terminal of X with Z Intact:

SEQ ID NO: 285

Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe;

SEQ ID NO: 286

Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe;

SEQ ID NO: 287

Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe;

SEQ ID NO: 288

Ala Ala Tyr Asp Lys Ala Ala Glu Lys Ala Lys Glu
Ala Ala Pro Asp Trp Leu Lys Ala Phe Tyr Asp Lys
Val Ala Glu Lys Leu Lys Glu Ala Phe;

SEQ ID NO: 289

Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala Phe;

SEQ ID NO: 290

Tyr Asp Lys Ala Ala Glu Lys Ala Lys Glu Ala Ala
Pro Asp Trp Leu Lys Ala Phe Tyr Asp Lys Val Ala
Glu Lys Leu Lys Glu Ala Phe;

5) Removal of Residues from the N Terminal of X and the N Terminal of Z:

SEQ ID NO: 291

Trp Ala Lys Ala Ala Tyr Asp Lys Ala Ala Glu Lys
Ala Lys Glu Ala Ala Pro Trp Leu Lys Ala Phe Tyr
Asp Lys Val Ala Glu Lys Leu Lys Glu Ala Phe;

SEQ ID NO: 292

Ala Lys Ala Ala Tyr Asp Lys Ala Ala Glu Lys Ala
Lys Glu Ala Ala Pro Leu Lys Ala Phe Tyr Asp Lys
Val Ala Glu Lys Leu Lys Glu Ala Phe;

SEQ ID NO: 293

Lys Ala Ala Tyr Asp Lys Ala Ala Glu Lys Ala Lys
Glu Ala Ala Pro Lys Ala Phe Tyr Asp Lys Val Ala
Glu Lys Leu Lys Glu Ala Phe;

SEQ ID NO: 294

Ala Ala Tyr Asp Lys Ala Ala Glu Lys Ala Lys Glu
Ala Ala Pro Ala Phe Tyr Asp Lys Val Ala Glu Lys
Leu Lys Glu Ala Phe;

SEQ ID NO: 295

Ala Tyr Asp Lys Ala Ala Glu Lys Ala Lys Glu Ala
Ala Pro Phe Tyr Asp Lys Val Ala Glu Lys Leu Lys
Glu Ala Phe;

SEQ ID NO: 296

Tyr Asp Lys Ala Ala Glu Lys Ala Lys Glu Ala Ala
Pro Tyr Asp Lys Val Ala Glu Lys Leu Lys Glu Ala
Phe;

6) Removal of Residues from the C Terminal of X and the C Terminal of Z:

SEQ ID NO: 239

Asp Trp Leu Lys Ala Phe Tyr Asp Lys Val Ala Glu
Lys Leu Lys Glu Ala Pro Asp Trp Ala Lys Ala Ala
Tyr Asp Lys Ala Ala Glu Lys Ala Lys Glu Ala;

SEQ ID NO: 240

Asp Trp Leu Lys Ala Phe Tyr Asp Lys Val Ala Glu
Lys Leu Lys Glu Pro Asp Trp Ala Lys Ala Ala Tyr
Asp Lys Ala Ala Glu Lys Ala Lys Glu;

SEQ ID NO: 241

Asp Trp Leu Lys Ala Phe Tyr Asp Lys Val Ala Glu
Lys Leu Lys Pro Asp Trp Ala Lys Ala Ala Tyr Asp
Lys Ala Ala Glu Lys Ala Lys;

SEQ ID NO: 242

Asp Trp Leu Lys Ala Phe Tyr Asp Lys Val Ala Glu
Lys Leu Pro Asp Trp Ala Lys Ala Ala Tyr Asp Lys
Ala Ala Glu Lys Ala;

SEQ ID NO: 243

Asp Trp Leu Lys Ala Phe Tyr Asp Lys Val Ala Glu
Lys Pro Asp Trp Ala Lys Ala Ala Tyr Asp Lys Ala
Ala Glu Lys;

SEQ ID NO: 244

Asp Trp Leu Lys Ala Phe Tyr Asp Lys Val Ala Glu
Pro Asp Trp Ala Lys Ala Ala Tyr Asp Lys Ala Ala
Glu;

SEQ ID NO: 245

Asp Trp Leu Lys Ala Phe Tyr Asp Lys Val Ala Pro
Asp Trp Ala Lys Ala Ala Tyr Asp Lys Ala Ala;

7) Removal of Residues from the C Terminal of Z and the N Terminal of X:

SEQ ID NO: 297

Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala;

SEQ ID NO: 298

Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu;

SEQ ID NO: 299

Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys;

SEQ ID NO: 300

Ala Ala Tyr Asp Lys Ala Ala Glu Lys Ala Lys Glu
Ala Ala Pro Asp Trp Leu Lys Ala Phe Tyr Asp Lys
Val Ala Glu Lys Leu;

SEQ ID NO: 301

Ala Tyr Asp Lys Ala Ala Glu Lys Ala Lys Glu Ala
Ala Pro Asp Trp Leu Lys Ala Phe Tyr Asp Lys Val
Ala Glu Lys;

SEQ ID NO: 302

Tyr Asp Lys Ala Ala Glu Lys Ala Lys Glu Ala Ala
Pro Asp Trp Leu Lys Ala Phe Tyr Asp Lys Val Ala
Glu;

8) Removal of Residues from the C Terminal of X and the N Terminal of Z:

SEQ ID NO: 303

Asp Trp Ala Lys Ala Ala Tyr Asp Lys Ala Ala Glu
Lys Ala Lys Glu Ala Pro Trp Leu Lys Ala Phe Tyr
Asp Lys Val Ala Glu Lys Leu Lys Glu Ala Phe;

SEQ ID NO: 304

Asp Trp Ala Lys Ala Ala Tyr Asp Lys Ala Ala Glu
Lys Ala Lys Glu Pro Leu Lys Ala Phe Tyr Asp Lys
Val Ala Glu Lys Leu Lys Glu Ala Phe;

SEQ ID NO: 305

Asp Trp Ala Lys Ala Ala Tyr Asp Lys Ala Ala Glu
Lys Ala Lys Pro Lys Ala Phe Tyr Asp Lys Val Ala
Glu Lys Leu Lys Glu Ala Phe;

SEQ ID NO: 306

Asp Trp Ala Lys Ala Ala Tyr Asp Lys Ala Ala Glu
Lys Ala Pro Ala Phe Tyr Asp Lys Val Ala Glu Lys
Leu Lys Glu Ala Phe;

SEQ ID NO: 307

Asp Trp Ala Lys Ala Ala Tyr Asp Lys Ala Ala Glu
Lys Pro Phe Tyr Asp Lys Val Ala Glu Lys Leu Lys
Glu Ala Phe;

SEQ ID NO: 308

Asp Trp Ala Lys Ala Ala Tyr Asp Lys Ala Ala Glu
Pro Tyr Asp Lys Val Ala Glu Lys Leu Lys Glu Ala
Phe;

Multiple Repeats of these orientations of X and Z.

(Xₙ-Y_{z}-Zₘ)ₛ

wherein: n is an integer from 1 to 3, m is an integer from 1 to 3, z is an integer from 1 to 13, and s is an integer from 1 to 5, wherein in some embodiments when s is more than 1, Y is optionally present between repeating units of X-Y-Z, and in some embodiments when n or m is greater than 1, Y is optionally present between repeats of X and/or between repeats of Z.

SEQ ID NO: 309

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Gly Lys Ala Lys Glu Ala Ala Pro Asp Trp Leu
Lys Ala Phe Tyr Asp Lys Val Ala Glu Lys Leu Lys
Glu Ala Phe;

SEQ ID NO: 310

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Gly Lys Ala Lys Glu Ala Ala Pro Asp
Trp Leu Lys ALa Phe Tyr Asp Lys Val Ala Glu Lys
Leu Lys Glu Ala Phe;

SEQ ID NO: 311

Asp Trp Ala Lys Ala Ala Tyr Asp Lys Ala Ala Glu
Lys Ala Lys Glu Ala Ala Pto Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro Asp Trp Leu Lys Ala Phe Tyr Asp Lys Val Ala
Glu Lys Leu Lys Glu Ala Phe;

SEQ ID NO: 312

Asp Trp Ala Lys Ala Ala Tyr Asp Lys Ala Ala Glu
Lys Ala Lys Glu Ala Ala Pro Asp Trp Ala Lys Ala
Ala Tyr Asp lys Ala Ala Glu Lys Ala Lys Glu Ala
Ala Pro Asp Trp Leu Lys Ala Phe Tyr Asp Lys Val
Ala Glu Lys Leu Lys Glu Ala Phe;

SEQ ID NO: 313

Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe Pro Asp Trp Leu Lys Ala Phe Tyr Asp Lys Val
Ala Glu Lys Leu Lys Glu Ala Phe;

d) reversal of the N to C orientation of the X in Xₙ-Y_{z}-Zₘ
when X is SEQ ID NO: 204 Asp Trp Leu Lys Ala Phe Tyr Asp Lys Val Ala Glu Lys Leu Lys Glu Ala Phe:

SEQ ID NO: 314

Phe Ala Glu Lys Leu Lys Glu Ala Val Lys Asp Tyr
Phe Ala Lys Leu Trp Asp Pro Asp Trp Ala Lys Ala
Ala Tyr Asp Lys Ala Ala Glu Lys Ala Lys Glu Ala
Ala

e) reversal of the N to C orientation of the Z in Xₙ-Y_{z}-Zₘ when Z is

SEQ ID NO: 205

Asp Trp Ala Lys Ala Ala Tyr Asp Lys Ala Ala Glu
Lys Ala Lys Glu Ala Ala;

SEQ ID NO: 315

Asp Trp Leu Lys Ala Phe Tyr Asp Lys Val Ala Glu
Lys Leu Lys Glu Ala Phe Pro Ala Ala Glu Lys Ala
Lys Glu Ala Ala Lys Asp Tyr Ala Ala Lys Ala Trp
Asp

f) reversal of the N terminal to C terminal orientation of Z and
reversal of the N terminal to C terminal orientation of X in Xₙ-Y_{z}-Zₘ:

SEQ ID NO: 316

Phe Ala Glu Lys Leu Lys Glu Ala Val Lys Asp Tyr
Phe Ala Lys Leu Trp Asp Pro Ala Ala Glu Lys Ala
Lys Glu Ala Ala Lys Asp Tyr Ala Ala Lys Ala Trp
Asp

g) reversal of the N terminal to C terminal orientation of Z and reversal of the N terminal to C terminal orientation of X in Xₙ-Y_{z}-Zₘ and change in the order, Z-Y-X:

SEQ ID NO: 317

Ala Ala Glu Lys Ala Lys Glu Ala Ala Lys Asp Tyr
Ala Ala Lys Ala Trp Asp Pro Phe Ala Glu Lys Leu
Lys Glu Ala Val Lys Asp Tyr Phe Ala Lys Leu Trp
Asp

h) reversal of the N terminal to C terminal orientation of Z when Z is SEQ ID NO: 204

SEQ ID NO: 204

Asp Trp Leu Lys Ala Phe Tyr Asp Lys Val Ala Glu
Lys Leu Lys Glu Ala Phe and X is

SEQ ID NO: 205

Asp Trp Ala Lys Ala Ala Tyr Asp Lys Ala Ala Glu
Lys Ala Lys Glu Ala Ala:

SEQ ID NO: 318

Asp Trp Ala Lys Ala Ala Tyr Asp Lys Ala Ala Glu
Lys Ala Lys Glu Ala Ala Pro Phe Ala Glu Lys Leu
Lys Glu Ala Val Lys Asp Tyr Phe Ala Lys Leu Trp
Asp

i) insertion of D amino acids in the one or more C-terminal six amino acids of Z, specifically SEQ ID NO: 207 Lys Ala Lys Glu Ala Ala.
Exemplary embodiments include but are not limited to the following:

SEQ ID NO: 319

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
ALA

SEQ ID NO: 320

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu ALA
Ala

SEQ ID NO: 321

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys GLU Ala
Ala

SEQ ID NO: 322

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala LYS Glu Ala
Ala

SEQ ID NO: 323

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu Ala
Ala

SEQ ID NO: 324

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala Lys Glu Ala
Ala

SEQ ID NO: 325

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu ALA
ALA

SEQ ID NO: 326

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu ALA
Ala

SEQ ID NO: 327

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu Ala
ALA

SEQ ID NO: 328

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS ALA Lys Glu Ala
ALA

SEQ ID NO: 329

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala LYS Glu Ala
ALA

SEQ ID NO: 330

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys GLU Ala
ALA

SEQ ID NO: 331

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala Lys GLU Ala
Ala

SEQ ID NO: 332

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala LYS Glu ALA
Ala

SEQ ID NO: 333

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys GLU Ala
ALA

It is to be understood that any of the peptides described above in the Xₙ-Y_{z}-Zₘ component may be combined with the D and/or D′ components described below and with the W and/or W′ components described below, or with other components of formulae VII-XVIII.

### Substitutions for D or D′

D or D′ is a peptide selected from the group consisting of SEQ ID NO: 15 Pro Arg Gly Gly Ser Val Leu Val Thr, or multiples, variations or conservative substitutions thereof. These amino acids may also appear in reverse orientation, namely SEQ ID NO: 16 Thr Val Leu Val Ser Gly Gly Arg Pro. It is to be understood that one or more of the first six N-terminal amino acids of D or D′, namely SEQ ID NO: 17 Pro Arg Gly Gly Ser Val or SEQ ID NO: 18 Thr Val Leu Val Ser Gly may occur as D-amino acids.

Conservative amino acid substitutions for amino acids have been described within this application. A few exemplary, non-limiting examples of such substitutions for SEQ ID NO: 15 Pro Arg Gly Gly Ser Val Leu Val Thr follow:

P—proline, leucine, valine, isoleucine or methionine;
R—arginine, lysine, valine, leucine, N-nitroarginine, β-cycloarginine, γ-hydroxy-arginine;
N-amidinocitruline or 2-amino-4-guanidino-butanoic acid;
G—glycine, serine, alanine, cysteine or threonine;
G—glycine, serine, alanine, cysteine or threonine;
V—valine, leucine, isoleucine or methionine;
L—leucine, isoleucine, methionine or valine;
V—valine, leucine, isoleucine or methionine;
T—threonine, serine, glycine, alanine or cysteine.
Exemplary embodiments for D or D′ include but are not limited to the following:

SEQ ID NO: 15

Pro Arg Gly Gly Ser Val Leu Val Thr;

SEQ ID NO: 16

Thr Val Leu Val Ser Gly Gly Arg Pro;

SEQ ID NO: 334

PRO Arg Gly Gly Ser Val Leu Val Thr;

SEQ ID NO: 335

Pro ARG Gly Gly Ser Val Leu Val Thr;

SEQ ID NO: 336

Pro Arg GLY Gly Ser Val Leu Val Thr;

SEQ ID NO: 337

Pro Arg Gly GLY Ser Val Leu Val Thr;

SEQ ID NO: 338

Pro Arg Gly Gly SER Val Leu Val Thr;

SEQ ID NO: 339

Pro Arg Gly Gly Ser VAL Leu Val Thr;

SEQ ID NO: 340

THR Val Leu Val Ser Gly Gly Arg Pro;

SEQ ID NO: 341

Thr VAL Leu Val Ser Gly Gly Arg Pro;

SEQ ID NO: 342

Thr Val LEU Val Ser Gly Gly Arg Pro;

SEQ ID NO: 343

Thr Val Leu VAL Ser Gly Gly Arg Pro;

SEQ ID NO: 344

Thr Val Leu Val SER Gly Gly Arg Pro;

SEQ ID NO: 345

Thr Val Leu Val Ser GLY Gly Arg Pro.

### Substitutions for W or W′

This section discloses an expansion of the peptides in the W and or W′ domains of Formula VII D-R-S-W-T-N-O-(Xₙ-Y_{z}-Zₘ)ₛ-O′-N′-T′-W′-S′-R′-D′)ᵣ and other formulae containing W or W′, wherein W or W′ is individually absent or present and is a peptide as defined in the present specification. In one embodiment, W or W′ is a peptide selected from the group consisting of SEQ ID NO: 21 Trp Arg Trp Trp Trp Trp, or multiples, variations or conservative substitutions thereof. These amino acids may also appear in reverse orientation, namely SEQ ID NO: 22 Trp Trp Trp Trp Arg Trp. It is to be understood that one or more of the amino acids in the W or W′ peptide may be D amino acids. While not wanting to be bound by the following statement, it is believed that W and/or W′ provide an anti-inflammatory component to the peptides of the present invention.

W or W′ may be any one of the following peptides: SEQ ID NO: 21 Trp Arg Trp Trp Trp Trp; SEQ ID NO: 346 Trp Trp Arg Trp Trp Trp; SEQ ID NO: 347 Trp Trp Trp Arg Trp Trp; SEQ ID NO: 22 Trp Trp Trp Trp Arg Trp; SEQ ID NO: 348 Trp Trp Trp Trp Trp Arg; SEQ ID NO: 349 Arg Trp Trp Trp Trp Trp, or multiples, variations or combinations thereof, including but not limited to the following,

SEQ ID NO: 350

Trp Arg Trp Trp Trp Trp Trp Arg Trp Trp Trp Trp

SEQ ID NO: 351

Trp Arg Trp Trp Trp Trp Trp Arg Trp Trp Trp Trp
Trp Arg Trp Trp Trp

SEQ ID NO: 352

Trp Trp Arg Trp Trp Trp Trp Trp Arg Trp Trp Trp

SEQ ID NO: 353

Trp Trp Arg Trp Trp Trp Trp Trp Arg Trp Trp Trp
Trp Trp Arg Trp Trp Trp

SEQ ID NO: 354

Trp Trp Trp Arg Trp Trp Trp Trp Trp Arg Trp Trp

SEQ ID NO: 355

Trp Trp Trp Arg Trp Trp Trp Trp Trp Arg Trp Trp
Trp Trp Trp Arg Trp Trp

SEQ ID NO: 356

Trp Trp Trp Trp Arg Trp Trp Trp Trp Trp Arg Trp

SEQ ID NO: 357

Trp Trp Trp Trp Arg Trp Trp Trp Trp Trp Arg Trp
Trp Trp Trp Trp Arg Trp

SEQ ID NO: 358

Trp Trp Trp Trp Trp Arg Trp Trp Trp Trp Trp Arg

SEQ ID NO: 359

Trp Trp Trp Trp Trp Arg Trp Trp Trp Trp Trp Arg
Trp Trp Trp Trp Trp Arg

SEQ ID NO: 360

Arg Trp Trp Trp Trp Trp Arg Trp Trp Trp Trp Trp

SEQ ID NO: 361

Arg Trp Trp Trp Trp Trp Arg Trp Trp Trp Trp Trp
Arg Trp Trp Trp Trp Trp

SEQ ID NO: 362

Trp Arg Trp Trp Trp Trp Trp Trp Arg Trp Trp Trp

SEQ ID NO: 363

Trp Arg Trp Trp Trp Trp Trp Arg Trp Trp Trp Trp
Trp Arg Trp Trp

SEQ ID NO: 364

Trp Arg Trp Trp Trp Trp Trp Trp Trp Trp Arg Trp

SEQ ID NO: 365

Trp Arg Trp Trp Trp Trp Trp Trp Trp Trp Arg Trp
Trp Trp Trp Trp Trp Arg.

The present invention includes substitution of amino acids in the peptides listed above. It is to be understood that tryptophan (W) may be conservatively substituted with alanine, phenylalanine, tyrosine or glycine in W or W′. It is also to be understood that arginine (R) may be substituted with lysine, valine or leucine in W or W′.

### Insertion of D Amino Acids in W or W′

Exemplary embodiments include but are not limited to the following:

| | |
|---|---|
| SEQ ID NO: 366 | TRP Arg Trp Trp Trp Trp; |
| SEQ ID NO: 367 | Trp ARG Trp Trp Trp Trp; |
| SEQ ID NO: 368 | Trp Arg TRP Trp Trp Trp; |
| SEQ ID NO: 369 | Trp Arg Trp TRP Trp Trp; |
| SEQ ID NO: 370 | Trp Arg Trp Trp TRP Trp; |
| SEQ ID NO: 371 | Trp Arg Trp Trp Trp TRP; |
| SEQ ID NO: 372 | TRP Trp Arg Trp Trp Trp; |
| SEQ ID NO: 373 | Trp TRP Arg Trp Trp Trp; |
| SEQ ID NO: 374 | Trp Trp ARG Trp Trp Trp; |
| SEQ ID NO: 375 | Trp Trp Arg TRP Trp Trp; |
| SEQ ID NO: 376 | Trp Trp Arg Trp TRP Trp; |
| SEQ ID NO: 377 | Trp Trp Arg Trp Trp TRP; |
| SEQ ID NO: 378 | TRP Trp Trp Arg Trp Trp; |
| SEQ ID NO: 379 | Trp TRP Trp Arg Trp Trp; |
| SEQ ID NO: 380 | Trp Trp TRP Arg Trp Trp; |
| SEQ ID NO: 381 | Trp Trp Trp ARG Trp Trp; |
| SEQ ID NO: 382 | Trp Trp Trp Arg TRP Trp; |
| SEQ ID NO: 383 | Trp Trp Trp Arg Trp TRP |

### Exemplary Embodiments for D-W (Formula XVI) and W-D (Formula XVII)

The following are non-limiting examples of D-W which is reversed in orientation for W-D (Formula XVII) and would apply to W′-D′ and D′-W′ as W′ may be equivalent to W and D′ may be equivalent to D.

SEQ ID NO: 384

Pro Arg Gly Gly Ser Val Leu Val Thr Trp Arg Trp
Trp Trp Trp;

SEQ ID NO: 385

PRO Arg Gly Gly Ser Val Leu Val Thr Trp Arg Trp
Trp Trp Trp;

SEQ ID NO: 386

Pro ARG Gly Gly Ser Val Leu Val Thr Trp Arg Trp
Trp Trp Trp;

SEQ ID NO: 387

Pro Arg GLY Gly Ser Val Leu Val Thr Trp Arg Trp
Trp Trp Trp;

SEQ ID NO: 388

Pro Arg Gly GLY Ser Val Leu Val Thr Trp Arg Trp
Trp Trp Trp;

SEQ ID NO: 389

Pro Arg Gly Gly SER Val Leu Val Thr Trp Arg Trp
Trp Trp Trp;

SEQ ID NO: 390

Pro Arg Gly Gly Ser VAL Leu Val Thr Trp Arg Trp
Trp Trp Trp;

SEQ ID NO: 391

Trp Arg Trp Trp Trp Trp Pro Arg Gly Gly Ser Val
Leu Val Thr;

SEQ ID NO: 392

Trp Arg Trp Trp Trp Trp PRO Arg Gly Gly Ser Val
Leu Val Thr;

SEQ ID NO: 393

Trp Arg Trp Trp Trp Trp Pro ARG Gly Gly Ser Val
Leu Val Thr;

SEQ ID NO: 394

Trp Arg Trp Trp Trp Trp Pro Arg GLY Gly Ser Val
Leu Val Thr;

SEQ ID NO: 395

Trp Arg Trp Trp Trp Trp Pro Arg Gly GLY Ser Val
Leu Val Thr;

SEQ ID NO: 396

Trp Arg Trp Trp Trp Trp Pro Arg Gly Gly SER Val
Leu Val Thr;

SEQ ID NO: 397

Arg Trp Trp Trp Trp Pro Arg Gly Gly Ser VAL Leu
Val Thr;

SEQ ID NO: 398

Pro Arg Gly Gly Ser Val Leu Val Thr TRP Arg Trp
Trp Trp Trp;

SEQ ID NO: 399

Pro Arg Gly Gly Ser Val Leu Val Thr Trp ARG Trp
Trp Trp Trp;

SEQ ID NO: 400

Pro Arg Gly Gly Ser Val Leu Val Thr Trp Arg TRP
Trp Trp Trp

SEQ ID NO: 401

Pro Arg Gly Gly Ser Val Leu Val Thr Trp Arg Trp
TRP Trp Trp;

SEQ ID NO: 402

Pro Arg Gly Gly Ser Val Leu Val Thr Trp Arg Trp
Trp TRP Trp;

SEQ ID NO: 403

Pro Arg Gly Gly Ser Val Leu Val Thr Trp Arg Trp
Trp Trp TRP

### Exemplary Embodiments of the Invention

The following peptides are exemplary embodiments of the present invention:

SEQ ID NO: 404

Trp Arg Trp Trp Trp Trp -(GABA)- Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala

SEQ ID NO: 405

Trp Arg Trp Trp Trp Trp (GABA-GABA)- Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala

SEQ ID NO: 406

Trp Arg Trp Trp Trp Trp (GABA-GABA-GABA)- Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala

SEQ ID NO: 407

Trp Arg Trp Trp Trp Trp (GABA-GABA-GABA-GABA) Pro
Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala

SEQ ID NO: 408

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala

SEQ ID NO: 409

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala

SEQ ID NO: 410

Trp Arg Trp Trp Trp Trp (GABA)- Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA)
Trp Arg Trp Trp Trp Trp;

SEQ ID NO: 411

Trp Arg Trp Trp Trp Trp -(GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA)- Trp Arg Trp Trp Trp Trp;

SEQ ID NO: 412

Trp Arg Trp Trp Trp Trp -(GABA-GABA-GABA)- Pro
Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABAGABA-GABA)- Trp Arg Trp Trp Trp Trp;

SEQ ID NO: 413

Trp Arg Trp Trp Trp Trp -(GABA-GABA-GABA-GABA)-
Pro Asp Trp Leu Lys Ala Phe Tyr Asp Lys Val Ala
Glu Lys Leu Lys Glu Ala Phe Pro Asp Trp Ala Lys
Ala Ala Tyr Asp Lys Ala Ala Glu Lys Ala Lys Glu
Ala Ala Pro (GABA-GABA-GABA-GABA)-Trp Arg Trp Trp
Trp Trp;

SEQ ID NO: 414

Trp Arg Trp Trp Trp Trp -(GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro
(GABA)-Trp Trp Trp Trp Arg Trp

SEQ ID NO: 415

Trp Arg Trp Trp Trp Trp -(GABA-GABA)- Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA) Trp Trp Trp Trp Arg Trp

SEQ ID NO: 416

Trp Arg Trp Trp Trp Trp -(GABA-GABA-GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA-GABA-GABA)-Trp Trp Trp Trp Arg Trp

SEQ ID NO: 417

Trp Arg Trp Trp Trp Trp -(GABA-GABA-GABA-GABA) Pro
Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA)-Trp Trp Trp Trp Arg
Trp

SEQ ID NO: 418

Trp Arg Trp Trp Trp Trp -(GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala (GABA)-Pro
Pro Arg Gly Gly Ser Val Leu Val Thr

SEQ ID NO: 419

TRP Arg Trp Trp Trp Trp -(GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala (GABA) Pro
Pro Arg Gly Gly Ser Val Leu Val THR

SEQ ID NO: 420

Trp Arg Trp Trp Trp Trp -(GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala (GABA) Thr
Val Leu Val Ser Gly Gly Arg Pro

SEQ ID NO: 421

TRP Arg Trp Trp Trp Trp -(GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala (GABA)-
Thr Val Leu Val Ser Gly Gly Arg PRO

### Series Including SEQ ID NO: 15 Pro Arg Gly Gly Ser Val Leu Val Thr

SEQ ID NO: 422

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 423

PRO ARG GLY GLY SER VAL LEU VAL THR (gaba) PRO TRP
ARG TRP TRP TRP TRP (gaba) ASP TRP LEU LYS ALA PHE
TYR ASP LYS VAL ALA GLU LYS LEU LYS GLU ALA PHE
PRO ASP TRP ALA LYS ALA ALA TYR ASP LYS ALA ALA
GLU LYS ALA LYS GLU ALA ALA;

SEQ ID NO: 424

PRO Arg Gly Gly Ser Val Leu Val Thr (GABA) Pro
Trp Arg Trp Trp Trp Trp (GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala ALA;

SEQ ID NO: 425

Pro Arg Gly Gly Ser Val Leu Val Thr (GABA) Pro Trp
Arg Trp Trp Trp Trp (GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala;

SEQ ID NO: 426

Pro Arg Gly Gly Ser Val Leu Val Thr (GABA) Pro Trp
Arg Trp Trp Trp Trp (GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala;

SEQ ID NO: 427

Pro Arg Gly Gly Ser Val Leu Val Thr (GABA) Pro Trp
Arg Trp Trp Trp Trp (GABA-GABA-GABA-GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala;

SEQ ID NO: 428

Pro Arg Gly Gly Ser Val Leu Val Thr (GABA) Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala Pro (GABA) Trp Arg Trp
Trp Trp Trp;

SEQ ID NO: 429

Pro Arg Gly Gly Ser Val Leu Val Thr (GABA) Pro Trp
Arg Trp Trp Trp Trp (GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA-
GABA) Trp Arg Trp Trp Trp Trp;

SEQ ID NO: 430

Pro Arg Gly Gly Ser Val Leu Val Thr (GABA) Pro Trp
Arg Trp Trp Trp Trp (GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA-GABA) Trp Arg Trp Trp Trp Trp;

SEQ ID NO: 431

Pro Arg Gly Gly Ser Val Leu Val Thr (GABA) Pro Trp
Arg Trp Trp Trp Trp (GABA-GABA-GABA-GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA-GABA-GABA-GABA) Trp Arg Trp Trp Trp Trp;

SEQ ID NO: 432

Pro Arg Gly Gly Ser Val Leu Val Thr (GABA) Pro Trp
Arg Trp Trp Trp Trp (GABA) Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala Pro (GABA) Trp Trp
Trp Trp Arg Trp;

SEQ ID NO: 433

Pro Arg Gly Gly Ser Val Leu Val Thr (GABA) Pro Trp
Arg Trp Trp Trp Trp (GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA-
GABA) Trp Trp Trp Trp Arg Trp;

SEQ ID NO: 434

Pro Arg Gly Gly Ser Val Leu Val Thr (GABA) Pro Trp
Arg Trp Trp Trp Trp (GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA-GABA) Trp Trp Trp Trp Arg Trp;

SEQ ID NO: 435

Pro Arg Gly Gly Ser Val Leu Val Thr (GABA) Pro Trp
Arg Trp Trp Trp Trp (GABA-GABA-GABA-GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA-GABA-GABA-GABA) Trp Trp Trp Trp Arg Trp;

SEQ ID NO: 436

Pro Arg Gly Gly Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala;

SEQ ID NO: 437

Pro Arg Gly Gly Ser Val Leu Val Thr Pro Trp Arg
Trp Trp Trp Trp (GABA-GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala Ala;

SEQ ID NO: 438

Pro Arg Gly Gly Ser Val Leu Val Thr Pro Trp Arg
Trp Trp Trp Trp (GABA-GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala;

SEQ ID NO: 439

Pro Arg Gly Gly Ser Val Leu Val Thr Pro Trp Arg
Trp Trp Trp Trp (GABA-GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala;

SEQ ID NO: 440

Pro Arg Gly Gly Ser Val Leu Val Thr Pro Trp Arg
Trp Trp Trp Trp (GABA)- Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala Pro (GABA) Trp Arg
Trp Trp Trp Trp;

SEQ ID NO: 441

Pro Arg Gly Gly Ser Val Leu Val Thr Pro Trp Arg
Trp Trp Trp Trp (GABA-GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala Ala Pro (GABA-
GABA) Trp Arg Trp Trp Trp Trp;

SEQ ID NO: 442

Pro Arg Gly Gly Ser Val Leu Val Thr Pro Trp Arg
Trp Trp Trp Trp (GABA-GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA-
GABA-GABA) Trp Arg Trp Trp Trp Trp;

SEQ ID NO: 443

Pro Arg Gly Gly Ser Val Leu Val Thr Pro Trp Arg
Trp Trp Trp Trp (GABA-GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA-GABA-GABA) Trp Arg Trp Trp Trp Trp;

SEQ ID NO: 444

Pro Arg Gly Gly Ser Val Leu Val Thr (GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala;

SEQ ID NO: 445

Pro Arg Gly Gly Ser Val Leu Val Thr (GABA-GABA)
Pro Asp Trp Leu Lys Ala Phe Tyr Asp Lys Val Ala
Glu Lys Leu Lys Glu Ala Phe Pro Asp Trp Ala Lys
Ala Ala Tyr Asp Lys Ala Ala Glu Lys Ala Lys Glu
Ala Ala;

SEQ ID NO: 446

Pro Arg Gly Gly Ser Val Leu Val Thr (GABA-GABA-
GABA) Pro Asp Trp Leu Lys Ala Phe Tyr Asp Lys Val
Ala Glu Lys Leu Lys Glu Ala Phe Pro Asp Trp Ala
Lys Ala Ala Tyr Asp Lys Ala Ala Glu Lys Ala Lys
Glu Ala Ala;

SEQ ID NO: 447

Pro Arg Gly Gly Ser Val Leu Val Thr (GABA-GABA-
GABA-GABA) Pro Asp Trp Leu Lys Ala Phe Tyr Asp Lys
Val Ala Glu Lys Leu Lys Glu Ala Phe Pro Asp Trp
Ala Lys Ala Ala Tyr Asp Lys Ala Ala Glu Lys Ala
Lys Glu Ala Ala;

SEQ ID NO: 448

Pro Arg Gly Gly Ser Val Leu Val Thr Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala;

SEQ ID NO: 449

Pro Arg Gly Gly Ser Val Leu Val Thr Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala;

SEQ ID NO: 450

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala Pro (GABA)- Trp
Arg Trp Trp Trp Trp Pro (GABA) Pro Arg Gly Gly Ser
Val Leu Val Thr;

SEQ ID NO: 451

Pro Arg Gly Gly Ser Val Leu Val Thr (GABA) Pro Trp
Arg Trp Trp Trp Trp (GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA-
GABA) Trp Arg Trp Trp Trp Trp Pro (GABA) Pro Arg
Gly Gly Ser Val Leu Val Thr;

SEQ ID NO: 452

Pro Arg Gly Gly Ser Val Leu Val Thr (GABA) Pro Trp
Arg Trp Trp Trp Trp (GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA-GABA) Trp Arg Trp Trp Trp Trp Pro
(GABA) Pro Arg Gly Gly Ser Val Leu Val Thr;

SEQ ID NO: 453

Pro Arg Gly Gly Ser Val Leu Val Thr (GABA) Pro Trp
Arg Trp Trp Trp Trp (GABA-GABA-GABA-GABA) Pro Asp
Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA-GABA-GABA-GABA) Trp Arg Trp Trp Trp Trp
Pro (GABA) Pro Arg Gly Gly Ser Val Leu Val Thr;

SEQ ID NO: 454

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Trp Arg Trp Trp Trp Trp;

SEQ ID NO: 455

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA-GABA) Trp Arg Trp Trp Trp Trp;

SEQ ID NO: 456

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Arg Trp Trp Trp Trp;

SEQ ID NO: 457

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Trp Trp Trp Trp Arg Trp;

SEQ ID NO: 458

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Trp Trp Trp Trp Arg Trp;

SEQ ID NO: 459

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Trp Trp Trp Arg Trp;

SEQ ID NO: 460

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Trp Trp Trp Trp Arg
Trp;

SEQ ID NO: 461

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Trp Trp Trp Trp Arg Trp;

SEQ ID NO: 462

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Trp Trp Trp Trp Arg Trp;

SEQ ID NO: 463

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Trp Arg Trp Trp Trp Trp Pro (GABA)
Pro Arg Gly Gly Ser Val Leu Val Thr;

SEQ ID NO: 464

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Trp Arg Trp Trp Trp Trp Pro
(GABA) Pro Arg Gly Gly Ser Val Leu Val Thr;

SEQ ID NO: 465

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Arg Trp Trp Trp Trp
Pro (GABA) Pro Arg Gly Gly Ser Val Leu Val Thr;

SEQ ID NO: 466

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Trp Arg Trp Trp Trp
Trp Pro (GABA) Pro Arg Gly Gly Ser Val Leu Val
Thr;

SEQ ID NO: 467

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Trp Arg Trp Trp Trp Trp Pro Pro Arg
Gly Gly Ser Val Leu Val Thr;

SEQ ID NO: 468

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Trp Arg Trp Trp Trp Trp Pro
Pro Arg Gly Gly Ser Val Leu Val Thr;

SEQ ID NO: 469

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Arg Trp Trp Trp Trp
Pro Pro Arg Gly Gly Ser Val Leu Val Thr;

SEQ ID NO: 470

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Trp Arg Trp Trp Trp
Trp Pro Pro Arg Gly Gly Ser Val Leu Val Thr;

### Inversion of SEQ ID NO: 15 Pro Arg Gly Gly Ser Val Leu Val Thr to be SEQ ID NO: 16 Thr Val Leu Val Ser Gly Gly Arg Pro

SEQ ID NO: 471

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Trp Arg Trp Trp Trp Trp Pro (GABA)
Thr Val Leu Val Ser Gly Gly Arg Pro;

SEQ ID NO: 472

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Trp Arg Trp Trp Trp Trp Pro
(GABA) Thr Val Leu Val Ser Gly Gly Arg Pro;

SEQ ID NO: 473

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Arg Trp Trp Trp Trp
Pro (GABA) Thr Val Leu Val Ser Gly Gly Arg Pro;

SEQ ID NO: 474

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Trp Arg Trp Trp Trp
Trp Pro (GABA) Thr Val Leu Val Ser Gly Gly Arg
Pro;

SEQ ID NO: 475

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu Ala Ala
Pro (GABA) Trp Arg Trp Trp Trp Trp Pro Thr Val Leu
Val Ser Gly Gly Arg Pro;

SEQ ID NO: 476

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Trp Arg Trp Trp Trp Trp Pro
Thr Val Leu Val Ser Gly Gly Arg Pro;

SEQ ID NO: 477

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Trp Arg Trp Trp Trp Trp
Pro Thr Val Leu Val Ser Gly Gly Arg Pro;

SEQ ID NO: 478

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Trp Arg Trp Trp Trp
Trp Pro Thr Val Leu Val Ser Gly Gly Arg Pro;

SEQ ID NO: 479

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Pro Arg Gly Gly Ser Val Leu Val
Thr;

SEQ ID NO: 480

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Pro Arg Gly Gly Ser Val Leu
Val Thr;

SEQ ID NO: 481

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Pro Arg Gly Gly Ser Val
Leu Val Thr;

SEQ ID NO: 482

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Pro Arg Gly Gly Ser
Val Leu Val Thr;

SEQ ID NO: 483

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala-(GABA) Pro Arg Gly Gly Ser Val Leu Val Thr;

SEQ ID NO: 484

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA) Pro Arg Gly Gly Ser Val Leu Val
Thr;

SEQ ID NO: 485

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA-GABA) Pro Arg Gly Gly Ser Val Leu
Val Thr;

SEQ ID NO: 486

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA-GABA-GABA) Pro Arg Gly Gly Ser Val
Leu Val Thr;

SEQ ID NO: 487

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Arg Gly Gly Ser Val Leu Val Thr;

SEQ ID NO: 488

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Pro Arg Gly Gly Ser Val Leu Val Thr;

SEQ ID NO: 489

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA) Thr Val Leu Val Ser Gly Gly Arg Pro

SEQ ID NO: 490

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA) Thr Val Leu Val Ser Gly Gly
Arg Pro;

SEQ ID NO: 491

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA) Thr Val Leu Val Ser Gly
Gly Arg Pro;

SEQ ID NO: 492

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro (GABA-GABA-GABA-GABA) Thr Val Leu Val Ser
Gly Gly Arg Pro;

SEQ ID NO: 493

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA) Thr Val Leu Val Ser Gly Gly Arg Pro

SEQ ID NO: 494

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA) Thr Val Leu Val Ser Gly Gly Arg
Pro;

SEQ ID NO: 495

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA-GABA) Thr Val Leu Val Ser Gly Gly
Arg Pro;

SEQ ID NO: 496

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala (GABA-GABA-GABA-GABA) Thr Val Leu Val Ser Gly
Gly Arg Pro;

SEQ ID NO: 497

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Thr Val Leu Val Ser Gly Gly Arg Pro;

SEQ ID NO: 498

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
Ala Pro Thr Val Leu Val Ser Gly Gly Arg Pro;

### Insertion of D Amino Acids in SEQ ID NO: 15 Pro Arg Gly Gly Ser Val Leu Val Thr

SEQ ID NO: 499

PRO Arg Gly Gly Ser Val Leu Val Thr (GABA) Pro Trp
Arg Trp Trp Trp Trp (GABA); Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala Ala;

SEQ ID NO: 500

Pro ARG Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 501

Pro Arg GLY Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 502

Pro Arg Gly GLY Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 503

Pro Arg Gly Gly SER Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 504

Pro Arg Gly Gly Ser VAL Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 505

PRO Arg Gly Gly Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala Pro (GABA) Trp Trp Trp
Trp Arg Trp;

SEQ ID NO: 506

Pro ARG Gly Gly Ser Val Leu Val Thr Pro Trp Arg
Trp Trp Trp Trp (GABA-GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala Ala Pro (GABA-
GABA) Trp Trp Trp Trp Arg Trp;

SEQ ID NO: 507

Pro Arg GLY Gly Ser Val Leu Val Thr Pro Trp Arg
Trp Trp Trp Trp (GABA-GABA-GABA) Pro Asp Trp Leu
Lys Ala Phe Tyr Asp Lys Val Ala Glu Lys Leu Lys
Glu Ala Phe Pro Asp Trp Ala Lys Ala Ala Tyr Asp
Lys Ala Ala Glu Lys Ala Lys Glu Ala Ala Pro (GABA-
GABA-GABA) Trp Trp Trp Trp Arg Trp;

SEQ ID NO: 508

Pro Arg Gly GLY Ser Val Leu Val Thr Pro Trp Arg
Trp Trp Trp Trp (GABA-GABA-GABA-GABA) Pro Asp Trp
Leu Lys Ala Phe Tyr Asp Lys Val Ala Glu Lys Leu
Lys Glu Ala Phe Pro Asp Trp Ala Lys Ala Ala Tyr
Asp Lys Ala Ala Glu Lys Ala Lys Glu Ala Ala Pro
(GABA-GABA-GABA-GABA) Trp Trp Trp Trp Arg Trp;

SEQ ID NO: 509

Pro Arg Gly Gly SER Val Leu Val Thr Pro Trp Arg
Trp Trp Trp Trp (GABA); Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala Ala;

SEQ ID NO: 510

Pro Arg Gly Gly Ser VAL Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala Ala;

SEQ ID NO: 511

PRO Arg Gly Gly Ser Val Leu Val Thr (GABA) Pro Trp
Arg Trp Trp Trp Trp;

SEQ ID NO: 512

Pro ARG Gly Gly Ser Val Leu Val Thr (GABA) Pro Trp
Arg Trp Trp Trp Trp;

SEQ ID NO: 513

Pro Arg GLY Gly Ser Val Leu Val Thr (GABA) Pro Trp
Arg Trp Trp Trp Trp;

SEQ ID NO: 514

Pro Arg Gly GLY Ser Val Leu Val Thr (GABA) Pro Trp
Arg Trp Trp Trp Trp;

SEQ ID NO: 515

Pro Arg Gly Gly SER Val Leu Val Thr (GABA) Pro Trp
Arg Trp Trp Trp Trp;

SEQ ID NO: 516

Pro Arg Gly Gly Ser VAL Leu Val Thr (GABA) Pro Trp
Arg Trp Trp Trp Trp;

SEQ ID NO: 517

Pro Arg Gly Gly Ser VAL Leu Val Thr Pro Trp Arg
Trp Trp Trp Trp

SEQ ID NO: 518

PRO Arg Gly Gly Ser Val Leu Val Thr Trp Arg Trp
Trp Trp Trp

SEQ ID NO: 519

Pro ARG Gly Gly Ser Val Leu Val Thr Trp Arg Trp
Trp Trp Trp

SEQ ID NO: 520

Pro Arg GLY Gly Ser Val Leu Val Thr Trp Arg Trp
Trp Trp Trp

SEQ ID NO: 521

Pro Arg Gly GLY Ser Val Leu Val Thr Trp Arg Trp
Trp Trp Trp

SEQ ID NO: 522

Pro Arg Gly Gly SER Val Leu Val Thr Trp Arg Trp
Trp Trp Trp

SEQ ID NO: 523

Pro Arg Gly Gly Ser VAL Leu Val Thr Trp Arg Trp
Trp Trp Trp

### Insertion of D Amino Acids in Z

SEQ ID NO: 524

Trp Arg Trp Trp Trp Trp (GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu Ala ALA;

SEQ ID NO: 525

Trp Arg Trp Trp Trp Trp (GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala Lys Glu ALA Ala;

SEQ ID NO: 526

Trp Arg Trp Trp Trp Trp (GABA) Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys GLU Ala Ala;

SEQ ID NO: 527

Trp Arg Trp Trp Trp Trp (GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys Ala LYS Glu Ala Ala;

SEQ ID NO: 528

Trp Arg Trp Trp Trp Trp (GABA) Pro Asp Trp Leu Lys
Ala Phe Tyr Asp Lys Val Ala Glu Lys Leu Lys Glu
Ala Phe Pro Asp Trp Ala Lys Ala Ala Tyr Asp Lys
Ala Ala Glu Lys ALA Lys Glu Ala Ala;

SEQ ID NO: 529

Trp Arg Trp Trp Trp Trp (GABA) Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu LYS Ala Lys Glu Ala Ala;

SEQ ID NO: 530

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala ALA;

SEQ ID NO: 531

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu ALA Ala;

SEQ ID NO: 532

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys GLU Ala Ala;

SEQ ID NO: 533

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala LYS Glu Ala Ala;

SEQ ID NO: 534

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys ALA Lys Glu Ala Ala

SEQ ID NO: 535

Trp Arg Trp Trp Trp Trp Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu LYS Ala Lys Glu Ala Ala

SEQ ID NO: 536

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala ALA

SEQ ID NO: 537

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu ALA Ala

SEQ ID NO: 538

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys GLU Ala Ala

SEQ ID NO: 539

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala LYS Glu Ala Ala

SEQ ID NO: 540

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys ALA Lys Glu Ala Ala

SEQ ID NO: 541

Trp Arg Trp Trp Trp Trp Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu LYS Ala Lys Glu Ala Ala

SEQ ID NO: 542

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
ALA Pro (GABA) Trp Arg Trp Trp Trp Trp;

SEQ ID NO: 543

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys Ala Lys Glu ALA Ala
Pro (GABA) Trp Arg Trp Trp Trp Trp;

SEQ ID NO: 544

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys GLU Ala
Ala Pro (GABA) Trp Arg Trp Trp Trp Trp;

SEQ ID NO: 545

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala LYS Glu Ala
Ala Pro (GABA) Trp Arg Trp Trp Trp Trp;

SEQ ID NO: 546

Trp Leu Lys Ala Phe Tyr Asp Lys Val Ala Glu Lys
Leu Lys Glu Ala Phe Pro Asp Trp Ala Lys Ala Ala
Tyr Asp Lys Ala Ala Glu Lys ALA Lys Glu Ala Ala
Pro (GABA) Trp Arg Trp Trp Trp Trp;

SEQ ID NO: 547

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala Lys Glu Ala
Ala Pro (GABA) Trp Arg Trp Trp Trp Trp;

SEQ ID NO: 548

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
ALA Pro Trp Arg Trp Trp Trp Trp;

SEQ ID NO: 549

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu ALA
Ala Pro Trp Arg Trp Trp Trp Trp;

SEQ ID NO: 550

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys GLU Ala
Ala Pro Trp Arg Trp Trp Trp Trp;

SEQ ID NO: 551

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala LYS Glu Ala
Ala Pro Trp Arg Trp Trp Trp Trp;

SEQ ID NO: 552

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu Ala
Ala Pro Trp Arg Trp Trp Trp Trp;

SEQ ID NO: 553

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala Lys Glu Ala
Ala Pro Trp Arg Trp Trp Trp Trp;

SEQ ID NO: 554

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu Ala
ALA Trp Arg Trp Trp Trp Trp;

SEQ ID NO: 555

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys Glu ALA
Ala Trp Arg Trp Trp Trp Trp;

SEQ ID NO: 556

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala Lys GLU Ala
Ala Trp Arg Trp Trp Trp Trp;

SEQ ID NO: 557

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys Ala LYS Glu Ala
Ala Trp Arg Trp Trp Trp Trp;

SEQ ID NO: 558

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu Lys ALA Lys Glu Ala
Ala Trp Arg Trp Trp Trp Trp;

SEQ ID NO: 559

Asp Trp Leu Lys Ala Phe Tyr Asp Lys Val Ala Glu
Lys Leu Lys Glu Ala Phe Pro Asp Trp Ala Lys Ala
Ala Tyr Asp Lys Ala Ala Glu LYS Ala Lys Glu Ala
Ala Trp Arg Trp Trp Trp Trp;

SEQ ID NO: 560

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu Ala ALA;

SEQ ID NO: 561

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu ALA Ala;

SEQ ID NO: 562

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys GLU Ala Ala;

SEQ ID NO: 563

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala LYS Glu Ala Ala;

SEQ ID NO: 564

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys ALA Lys Glu Ala Ala;

SEQ ID NO: 565

Pro Arg Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu LYS Ala Lys Glu Ala Ala;

### Insertion of D Amino Acids in SEQ ID NO: 15 Pro Arg Gly Gly Ser Val Leu Val Thr and Insertion of D Amino Acids in Z

SEQ ID NO: 566

Pro ARG Gly Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys Glu ALA Ala;

SEQ ID NO: 567

Pro Arg GLY Gly Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala Lys GLU Ala Ala;

SEQ ID NO: 568

Pro Arg Gly GLY Ser Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys Ala LYS Glu Ala Ala;

SEQ ID NO: 569

Pro Arg Gly Gly SER Val Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu Lys ALA Lys Glu Ala Ala;

SEQ ID NO: 570

Pro Arg Gly Gly Ser VAL Leu Val Thr (GABA) Pro Trp
Arg Trp Trp Trp Trp (GABA) Pro Asp Trp Leu Lys Ala
Phe Tyr Asp Lys Val Ala Glu Lys Leu Lys Glu Ala
Phe Pro Asp Trp Ala Lys Ala Ala Tyr Asp Lys Ala
Ala Glu LYS Ala Lys Glu Ala Ala;

SEQ ID NO: 571

PRO Arg Gly Gly Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu Ala ALA;

SEQ ID NO: 572

Pro ARG Gly Gly Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys Glu ALA Ala;

SEQ ID NO: 573

Pro Arg GLY Gly Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala Lys GLU Ala Ala;

SEQ ID NO: 574

Pro Arg Gly GLY Ser Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys Ala LYS Glu Ala Ala;

SEQ ID NO: 575

Pro Arg Gly Gly SER Val Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu Lys ALA Lys Glu Ala Ala;

SEQ ID NO: 576

Pro Arg Gly Gly Ser VAL Leu Val Thr Pro Trp Arg
Trp Trp Trp Trp (GABA) Pro Asp Trp Leu Lys Ala Phe
Tyr Asp Lys Val Ala Glu Lys Leu Lys Glu Ala Phe
Pro Asp Trp Ala Lys Ala Ala Tyr Asp Lys Ala Ala
Glu LYS Ala Lys Glu Ala Ala;

### Other Exemplary Embodiments of the Present Invention

SEQ ID NO: 577

Pro Arg Gly Gly Ser Val Leu Val Thr (GABA) Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe Pro
Trp Arg Trp Trp Trp Trp;

SEQ ID NO: 578

Pro Arg Gly Gly Ser Val Leu Val Thr (GABA)-(GABA)
Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe Pro Trp Arg Trp Trp Trp Trp;

SEQ ID NO: 579

Pro Arg Gly Gly Ser Val Leu Val Thr (GABA)-(GABA)-
(GABA) Asp Trp Ala Lys Ala Ala Tyr Asp Lys Ala Ala
Glu Lys Ala Lys Glu Ala Ala Pro Asp Trp Leu Lys
Ala Phe Tyr Asp Lys Val Ala Glu Lys Leu Lys Glu
Ala Phe Pro Trp Arg Trp Trp Trp Trp;

SEQ ID NO: 580

Pro Arg Gly Gly Ser Val Leu Val Thr (GABA)-(GABA)-
(GABA)-(GABA) Asp Trp Ala Lys Ala Ala Tyr Asp Lys
Ala Ala Glu Lys Ala Lys Glu Ala Ala Pro Asp Trp
Leu Lys Ala Phe Tyr Asp Lys Val Ala Glu Lys Leu
Lys Glu Ala Phe Pro Trp Arg Trp Trp Trp Trp;

SEQ ID NO: 581

Pro Arg Gly Gly Ser Val Leu Val Thr Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Trp Arg
Trp Trp Trp Trp;

SEQ ID NO: 582

Pro Arg Gly Gly Ser Val Leu Val Thr Pro Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe Pro
Trp Arg Trp Trp Trp Trp;

SEQ ID NO: 583

Pro Arg Gly Gly Ser Val Leu Val Thr (GABA) Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe (GABA)
Trp Arg Trp Trp Trp Trp;

SEQ ID NO: 584

Pro Arg Gly Gly Ser Val Leu Val Thr (GABA) Pro Trp
Arg Trp Trp Trp Trp (GABA) Pro Asp Trp Ala Lys Ala
Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala Phe;

SEQ ID NO: 585

PRO Arg Gly Gly Ser Val Leu Val Thr (GABA) Pro Trp
Arg Trp Trp Trp Trp (GABA) Pro Asp Trp Ala Lys Ala
Ala Tyr Asp Lys Ala Ala Glu Lys Ala Lys Glu Ala
Ala Pro Asp Trp Leu Lys Ala Phe Tyr Asp Lys Val
Ala Glu Lys Leu Lys Glu Ala PHE;

SEQ ID NO: 586

Pro Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe
(GABA) Pro Trp Arg Trp Trp Trp Trp;

SEQ ID NO: 587

PRO Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe
(GABA) Pro Trp Arg Trp Trp Trp TRP;

SEQ ID NO: 588

Pro Arg Gly Gly Ser Val Leu Val Thr (GABA)-(GABA)
Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe (GABA)-(GABA) Trp Arg Trp Trp Trp Trp;

SEQ ID NO: 589

Pro Arg Gly Gly Ser Val Leu Val Thr (GABA)-(GABA)-
(GABA) Asp Trp Ala Lys Ala Ala Tyr Asp Lys Ala Ala
Glu Lys Ala Lys Glu Ala Ala Pro Asp Trp Leu Lys
Ala Phe Tyr Asp Lys Val Ala Glu Lys Leu Lys Glu
Ala Phe (GABA)-(GABA)-(GABA) Trp Arg Trp Trp Trp
Trp;

SEQ ID NO: 590

Pro Arg Gly Gly Ser Val Leu Val Thr (GABA)-(GABA)-
(GABA)-(GABA) Asp Trp Ala Lys Ala Ala Tyr Asp Lys
Ala Ala Glu Lys Ala Lys Glu Ala Ala Pro Asp Trp
Leu Lys Ala Phe Tyr Asp Lys Val Ala Glu Lys Leu
Lys Glu Ala Phe (GABA)-(GABA)-(GABA)-(GABA) Trp
Arg Trp Trp Trp Trp;

SEQ ID NO: 591

Pro Arg Gly Gly Ser Val Leu Val Thr Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Trp Arg
Trp Trp Trp Trp;

SEQ ID NO: 592

Pro Arg Gly Gly Ser Val Leu Val Thr (GABA) Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe Pro
Trp Trp Trp Trp Arg Trp;

SEQ ID NO: 593

Pro Arg Gly Gly Ser Val Leu Val Thr (GABA)-(GABA)
Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe Pro Trp Trp Trp Trp Arg Trp;

SEQ ID NO: 594

Pro Arg Gly Gly Ser Val Leu Val Thr (GABA)-(GABA)-
(GABA) Asp Trp Ala Lys Ala Ala Tyr Asp Lys Ala Ala
Glu Lys Ala Lys Glu Ala Ala Pro Asp Trp Leu Lys
Ala Phe Tyr Asp Lys Val Ala Glu Lys Leu Lys Glu
Ala Phe Pro Trp Trp Trp Trp Arg Trp;

SEQ ID NO: 595

Pro Arg Gly Gly Ser Val Leu Val Thr (GABA)-(GABA)-
(GABA)-(GABA) Asp Trp Ala Lys Ala Ala Tyr Asp Lys
Ala Ala Glu Lys Ala Lys Glu Ala Ala Pro Asp Trp
Leu Lys Ala Phe Tyr Asp Lys Val Ala Glu Lys Leu
Lys Glu Ala Phe Pro Trp Trp Trp Trp Arg Trp;

SEQ ID NO: 596

Pro Arg Gly Gly Ser Val Leu Val Thr Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Trp Trp
Trp Trp Arg Trp;

SEQ ID NO: 597

Pro Arg Gly Gly Ser Val Leu Val Thr Pro Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe Pro
Trp Trp Trp Trp Arg Trp;

SEQ ID NO: 598

Pro Arg Gly Gly Ser Val Leu Val Thr (GABA) Asp Trp
Ala Lys Ala Ala Tyr Asp Lys Ala Ala Glu Lys Ala
Lys Glu Ala Ala Pro Asp Trp Leu Lys Ala Phe Tyr
Asp Lys Val Ala Glu Lys Leu Lys Glu Ala Phe
(GABA)- Trp Trp Trp Trp Arg Trp;

SEQ ID NO: 599

Pro Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe-
(GABA) Pro Trp Trp Trp Trp Arg Trp;

SEQ ID NO: 600

PRO Arg Gly Gly Ser Val Leu Val Thr Pro (GABA) Asp
Trp Ala Lys Ala Ala Tyr Asp Lys Ala Ala Glu Lys
Ala Lys Glu Ala Ala Pro Asp Trp Leu Lys Ala Phe
Tyr Asp Lys Val Ala Glu Lys Leu Lys Glu Ala Phe
(GABA) Pro Trp Trp Trp Trp Arg TRP;

SEQ ID NO: 601

Pro Arg Gly Gly Ser Val Leu Val Thr (GABA)-(GABA)
Asp Trp Ala Lys Ala Ala Tyr Asp Lys Ala Ala Glu
Lys Ala Lys Glu Ala Ala Pro Asp Trp Leu Lys Ala
Phe Tyr Asp Lys Val Ala Glu Lys Leu Lys Glu Ala
Phe (GABA)-(GABA) Trp Trp Trp Trp Arg Trp;

SEQ ID NO: 602

Pro Arg Gly Gly Ser Val Leu Val Thr (GABA)-(GABA)-
(GABA) Asp Trp Ala Lys Ala Ala Tyr Asp Lys Ala Ala
Glu Lys Ala Lys Glu Ala Ala Pro Asp Trp Leu Lys
Ala Phe Tyr Asp Lys Val Ala Glu Lys Leu Lys Glu
Ala Phe (GABA)-(GABA)-(GABA)- Trp Trp Trp Trp Arg
Trp;

SEQ ID NO: 603

Pro Arg Gly Gly Ser Val Leu Val Thr (GABA)-(GABA)-
(GABA)-(GABA)- Asp Trp Ala Lys Ala Ala Tyr Asp Lys
Ala Ala Glu Lys Ala Lys Glu Ala Ala Pro Asp Trp
Leu Lys Ala Phe Tyr Asp Lys Val Ala Glu Lys Leu
Lys Glu Ala Phe (GABA)-(GABA)-(GABA)-(GABA) Trp
Trp Trp Trp Arg Trp;

SEQ ID NO: 604

Pro Arg Gly Gly Ser Val Leu Val Thr Asp Trp Ala
Lys Ala Ala Tyr Asp Lys Ala Ala Glu Lys Ala Lys
Glu Ala Ala Pro Asp Trp Leu Lys Ala Phe Tyr Asp
Lys Val Ala Glu Lys Leu Lys Glu Ala Phe Trp Trp
Trp Trp Arg Trp;

SEQ ID NO: 605

Pro Arg Gly Gly Ser Val Leu Val Thr Asp Trp Ala
Lys Ala Ala Tyr Asp Lys Ala Ala Glu Lys Ala Lys
Glu Ala Ala Pro Phe Ala Glu Lys Leu Lys Glu Ala
Val Lys Asp Tyr Phe Ala Lys Leu Trp Asp Trp Arg
Trp Trp Trp Trp;

SEQ ID NO: 606

Pro Arg Gly Gly Ser Val Leu Val Thr Pro Asp Trp
Ala Lys Ala Ala Tyr Asp Lys Ala Ala Glu Lys Ala
Lys Glu Ala Ala Pro Phe Ala Glu Lys Leu Lys Glu
Ala Val Lys Asp Tyr Phe Ala Lys Leu Trp Asp Pro
Trp Arg Trp Trp Trp Trp;

SEQ ID NO: 607

Pro Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Phe Ala Glu Lys Leu Lys
Glu Ala Val Lys Asp Tyr Phe Ala Lys Leu Trp Asp
(GABA) Pro Trp Arg Trp Trp Trp Trp;

SEQ ID NO: 608

PRO Arg Gly Gly Ser Val Leu Val Thr Asp Trp Ala
Lys Ala Ala Tyr Asp Lys Ala Ala Glu Lys Ala Lys
Glu Ala Ala Pro Phe Ala Glu Lys Leu Lys Glu Ala
Val Lys Asp Tyr Phe Ala Lys Leu Trp Asp Trp Arg
Trp Trp Trp TRP;

SEQID NO: 609

PRO Arg Gly Gly Ser Val Leu Val Thr Pro Asp Trp
Ala Lys Ala Ala Tyr Asp Lys Ala Ala Glu Lys Ala
Lys Glu Ala Ala Pro Phe Ala Glu Lys Leu Lys Glu
Ala Val Lys Asp Tyr Phe Ala Lys Leu Trp Asp Pro
Trp Arg Trp Trp Trp TRP;

SEQ ID NO: 610

PRO Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Phe Ala Glu Lys Leu Lys
Glu Ala Val Lys Asp Tyr Phe Ala Lys Leu Trp Asp
(GABA) Pro Trp Arg Trp Trp Trp TRP;

SEQ ID NO: 611

Pro Arg Gly Gly Ser Val Leu Val Thr Asp Trp Ala
Lys Ala Ala Tyr Asp Lys Ala Ala Glu Lys Ala Lys
Glu Ala Ala Pro Phe Ala Glu Lys Leu Lys Glu Ala
Val Lys Asp Tyr Phe Ala Lys Leu Trp Asp Trp Trp
Trp Trp Arg Trp;

SEQ ID NO: 612

Pro Arg Gly Gly Ser Val Leu Val Thr Pro Asp Trp
Ala Lys Ala Ala Tyr Asp Lys Ala Ala Glu Lys Ala
Lys Glu Ala Ala Pro Phe Ala Glu Lys Leu Lys Glu
Ala Val Lys Asp Tyr Phe Ala Lys Leu Trp Asp Pro
Trp Trp Trp Trp Arg Trp;

SEQ ID NO: 613

Pro Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Phe Ala Glu Lys Leu Lys
Glu Ala Val Lys Asp Tyr Phe Ala Lys Leu Trp Asp
(GABA) Pro Trp Trp Trp Trp Arg Trp;

SEQ ID NO: 614

PRO Arg Gly Gly Ser Val Leu Val Thr Asp Trp Ala
Lys Ala Ala Tyr Asp Lys Ala Ala Glu Lys Ala Lys
Glu Ala Ala Pro Phe Ala Glu Lys Leu Lys Glu Ala
Val Lys Asp Tyr Phe Ala Lys Leu Trp Asp Trp Trp
Trp Trp Arg TRP;

SEQ ID NO: 615

PRO Arg Gly Gly Ser Val Leu Val Thr Pro Asp Trp
Ala Lys Ala Ala Tyr Asp Lys Ala Ala Glu Lys Ala
Lys Glu Ala Ala Pro Phe Ala Glu Lys Leu Lys Glu
Ala Val Lys Asp Tyr Phe Ala Lys Leu Trp Asp Pro
Trp Trp Trp Trp Arg TRP;

SEQ ID NO: 616

PRO Arg Gly Gly Ser Val Leu Val Thr (GABA) Pro Asp
Trp Ala Lys Ala Ala Tyr Asp Lys Ala Ala Glu Lys
Ala Lys Glu Ala Ala Pro Phe Ala Glu Lys Leu Lys
Glu Ala Val Lys Asp Tyr Phe Ala Lys Leu Trp Asp
(GABA) Pro Trp Trp Trp Trp Arg TRP.

It is to be understood that the letters in the generic formulae I through XVIII or in components thereof are defined by the text that follows each letter and do not designate an individual amino acid.

It is to be understood that in some embodiments, one or more of the amino acids of the peptides of the present invention are D amino acids. In one embodiment, the N-terminal amino acid, the C-terminal amino acid or both are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

N-Terminal Modification and/or C-Terminal Modification of the Peptides of the Present Invention

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art. While it is to be understood that any of the peptides disclosed in this application may be modified at the N-terminus, at the C-terminus, or both at the N-terminus and at the C-terminus, the following sequences are presented as exemplary embodiments.

SEQ ID NO: 617

Ac-Ser Pro Leu Gly Glu Glu Met Arg Asp Arg Ala Arg
Ala His Val Asp Ala Leu Arg Thr His Lys Leu Ser
Pro Leu Leu Glu Ser Phe Lys Val Ser Phe Leu Ser
Ala Leu Glu Glu Tyr Thr Lys Lys Leu Asn Thr Gln-
NH₂;

SEQ ID NO: 618

Ac-Ser Pro Leu Ser Asp Glu Leu Arg Gln Arg Leu
Ala Ala Arg Leu Glu Ala Leu Lys Glu Asn Lys Leu
Ser Pro Leu Leu Glu Ser Phe Lys Val Ser Phe Leu
Ser Ala Leu Glu Glu Tyr Thr Lys Lys Leu Asn Thr
Gln-NH₂;

SEQ ID NO: 619

Ac-Ser Asp Glu Leu Arg Gln Arg Leu Ala Ala Arg Leu
Glu Ala Leu Lys Glu Asn Pro Leu Glu Ser Phe Lys
Val Ser Phe Leu Ser Ala Leu Glu Glu Tyr Thr Lys
Lys-NH₂;

SEQ ID NO: 620

Ac-Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp-NH₂;

SEQ ID NO: 621

Ac-Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Lys Leu Ser Pro
Leu Leu Glu Ser Phe Lys Val Ser Phe Leu Ser Ala
Leu Glu Glu Tyr Thr Lys Lys Leu Asn Thr Gln-NH₂;

SEQ ID NO: 622

Ac-Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp-NH₂;

SEQ ID NO: 623

Ac-Ser Pro Leu Gly Glu Glu Met Arg Asp Arg Ala Arg
Ala His Val Asp Ala Leu Arg Thr His Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp-NH₂;

SEQ ID NO: 624

Ac-Ser Pro Leu Leu Glu Ser Ala Lys Val Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys Leu Asn Thr Gln-
NH₂;

SEQ ID NO: 625

Ac-Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Lys Leu Ser Pro
Leu Leu Glu Ser Phe Lys Val Ser Phe Leu Ser Ala
Leu Glu Glu Tyr Thr Lys Lys Leu Asn Thr Gln-NH₂;

SEQ ID NO: 626

Ac-Ser Pro Leu Leu Glu Ser Ala Lys Val Ser Ala Leu
Ser Ala Leu Glu Glu Ala Thr Lys Lys Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp-NH₂;

SEQ ID NO: 627

Ac-Leu Glu Ser Ala Lys Val Ser Ala Leu Ser Ala Leu
Glu Glu Ala Thr Lys Lys Lys Leu Ser Pro Leu Leu
Glu Ser Phe Lys Val Ser Phe Leu Ser Ala Leu Glu
Glu Tyr Thr Lys Lys-NH₂;

SEQ ID NO: 628

Ac-Leu Glu Ser Ala Lys Val Ser Ala Leu Ser Ala Leu
Glu Glu Ala Thr Lys Lys Pro Leu Glu Ser Phe Lys
Val Ser Phe Leu Ser Ala Leu Glu Glu Tyr Thr Lys
Lys Lys-NH₂.

### Modified Peptides of the Present Invention

The present invention may be used for the production of the peptides or peptide analogs of the present invention. “Proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the protein, or to the alpha-amino group (imino group when participating in a peptide bond) of an amino acid at any other location within the protein. Similarly, the term “carboxy terminus” (abbreviated C-terminus) refers to the free carboxyl group on the amino acid at the carboxy terminus of a protein, or to the carboxyl group of an amino acid at any other location within the protein.

Typically, the amino acids making up a protein are numbered in order, starting at the amino terminal and increasing in the direction toward the carboxy terminal of the protein. Thus, when one amino acid is said to “follow” another, that amino acid is positioned closer to the carboxy terminal of the protein than the preceding amino acid.

The term “residue” is used herein to refer to an amino acid (D or L) or an amino acid mimetic that is incorporated into a protein by an amide bond. When a D amino acid is present in the peptides of the present invention, the three letter designation for the amino acid appears in upper case instead of a capital letter. For example the amino acid serine, represented as Ser indicates an L amino acid. The D amino acid form is represented as the upper case letters SER. This is not to be confused with letters appearing as subscripts used in generic formula and defined as variables herein. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one of skill will recognize that, as mentioned above, individual substitutions, deletions or additions which alter, add or delete a single amino acid or a small percentage of amino acids (typically less than about 5%, or typically less than about 1%) in a sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q), Histidine (H);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” also refers to utilizing a substituting amino acid which is identical to the amino acid being substituted except that a functional group in the side chain is protected with a suitable protecting group. Peptides of the present invention include conservatively substituted peptides, wherein these conservative substitutions occur at 1%, 3%, 5%, 7%, 10%, 15%, 20%, 25%, 30%, 40%, or 50% of the amino acid residues. Peptides of the present invention include peptides that are homologous at 50%, 60%, 70%, 80%, 90%, 95%, 97%, 98%, 99% of the entire sequence of the peptide.

Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those which facilitate transport of the peptide through membranes, for example, by reducing the hydrophilicity and increasing the lipophilicity of the peptide, and which can be cleaved, either by hydrolysis or enzymatically (Ditter et al., 1968. J. Pharm. Sci. 57:783; Ditter et al., 1968. J. Pharm. Sci. 57:828; Ditter et al., 1969. J. Pharm. Sci. 58:557; King et al., 1987. Biochemistry 26:2294; Lindberg et al., 1989. Drug Metabolism and Disposition 17:311; Tunek et al., 1988. Biochem. Pharm. 37:3867; Anderson et al., 1985 Arch. Biochem. Biophys. 239:538; and Singhal et al., 1987. FASEB J. 1:220). Suitable hydroxyl protecting groups include ester, carbonate and carbamate protecting groups. Suitable amine protecting groups include acyl groups and alkoxy or aryloxy carbonyl groups, as described above for N-terminal protecting groups. Suitable carboxylic acid protecting groups include aliphatic, benzyl and aryl esters, as described below for C-terminal protecting groups. In one embodiment, the carboxylic acid group in the side chain of one or more glutamic acid or aspartic acid residues in a peptide of the present invention is protected, preferably as a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting, i.e. that there are additional modified amino acids which could be included in each group.
Group I includes leucine, isoleucine, valine, methionine and modified amino acids having the following side chains: ethyl, n-propyl n-butyl. Preferably, Group I includes leucine, isoleucine, valine and methionine.
Group II includes glycine, alanine, valine and a modified amino acid having an ethyl side chain. Preferably, Group II includes glycine and alanine.
Group III includes phenylalanine, phenylglycine, tyrosine, tryptophan, cyclohexylmethyl glycine, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.
Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, —CO—NH— alkylated glutamine or asparagines (e.g., methyl, ethyl, n-propyl and iso-propyl) and modified amino acids having the side chain-(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.
Group V includes histidine, lysine, ornithine, arginine, N-nitroarginine, β-cycloarginine, γ-hydroxyarginine, N-amidinocitruline and 2-amino-4-guanidinobutanoic acid, homologs of lysine, homologs of arginine and homologs of ornithine. Preferably, Group V includes histidine, lysine, arginine and ornithine. A homolog of an amino acid includes from 1 to about 3 additional or subtracted methylene units in the side chain.
Group VI includes serine, threonine, and modified amino acids having C1-C5 straight or branched alkyl side chains substituted with —OH or —SH, for example, —CH₂CH₂OH, —CH₂CH₂CH₂OH or —CH₂CH₂OHCH₃. Preferably, Group VI includes serine, or threonine.

In another aspect, suitable substitutions for amino acid residues include “severe” substitutions. A “severe substitution” is a substitution in which the substituting amino acid (naturally occurring or modified) has significantly different size and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cyclohexylmethyl glycine for alanine, isoleucine for glycine, a D amino acid for the corresponding L amino acid, or —NH—CH[(—CH₂)₅—COOH]—CO— for aspartic acid. Alternatively, a functional group may be added to the side chain, deleted from the side chain or exchanged with another functional group. Examples of severe substitutions of this type include adding of valine, leucine or isoleucine, exchanging the carboxylic acid in the side chain of aspartic acid or glutamic acid with an amine, or deleting the amine group in the side chain of lysine or ornithine. In yet another alternative, the side chain of the substituting amino acid can have significantly different steric and electronic properties that the functional group of the amino acid being substituted. Examples of such modifications include tryptophan for glycine, lysine for aspartic acid and —(CH₂)₄COOH for the side chain of serine. These examples are not meant to be limiting.

In addition to the naturally occurring genetically encoded amino acids, amino acid residues in the peptides may be substituted with naturally occurring non-encoded amino acids and synthetic amino acids. Certain commonly encountered amino acids which provide useful substitutions include, but are not limited to, β-alanine and other omega-amino acids, such as 3-aminopropionic acid, 2,3-diaminopropionic acid, 4-aminobutyric acid and the like; α-aminoisobutyric acid; ε-aminohexanoic acid; δ-aminovaleric acid; N-methylglycine or sarcosine; ornithine; citrulline; t-butylalanine; t-butylglycine; N-methylisoleucine; phenylglycine; cyclohexylalanine; norleucine; naphthylalanine; 4-chlorophenylalanine; 2-fluorophenylalanine; 3-fluorophenylalanine; 4-fluorophenylalanine; penicillamine; 1,2,3,4-tetrahydroisoquinoline-3-carboxylic acid; β2-thienylalanine; methionine sulfoxide; homoarginine; N-acetyl lysine; 2,4-diaminobutyric acid; 2,3-diaminobutyric acid; p-aminophenylalanine; N-methyl valine; homocysteine; homophenylalanine; homoserine; hydroxyproline; homoproline; N-methylated amino acids; and peptoids (N-substituted glycines).

While in certain embodiments, the amino acids of the peptides will be substituted with L-amino acids, the substitutions are not limited to L-amino acids. Thus, also encompassed by the present disclosure are modified forms of the peptides, wherein an L-amino acid is replaced with an identical D-amino acid (e.g., L-Arg→D-Arg) or with a conservatively-substituted D-amino acid (e.g., LArg→D-Lys), and vice versa.

Additional aspects of the disclosure include analogs, variants, derivatives, and mimetics based on the amino acid sequence of the peptides disclosed herein. Typically, mimetic compounds are synthetic compounds having a three-dimensional structure (of at least part of the mimetic compound) that mimics, for example, the primary, secondary, and/or tertiary structural, and/or electrochemical characteristics of a selected peptide, structural domain, active site, or binding region (e.g., a homotypic or heterotypic binding site, a catalytic active site or domain, a receptor or ligand binding interface or domain, or a structural motif) thereof. The mimetic compound will often share a desired biological activity with a native peptide, as discussed herein (e.g., the ability to interact with lipids). Typically, at least one subject biological activity of the mimetic compound is not substantially reduced in comparison to, and is often the same as or greater than, the activity of the native peptide on which the mimetic was modeled.

A variety of techniques well known to one of skill in the art are available for constructing synthetic peptide mimetics with the same, similar, increased, or reduced biological activity as the corresponding native peptide. Often these analogs, variants, derivatives and mimetics will exhibit one or more desired activities that are distinct or improved from the corresponding native peptide, for example, improved characteristics of solubility, stability, lipid interaction, and/or susceptibility to hydrolysis or proteolysis (see, e.g., Morgan and Gainor, Ann. Rep. Med. Chem. 24:243-252, 1989). In addition, mimetic compounds of the disclosure can have other desired characteristics that enhance their therapeutic application, such as increased cell permeability, greater affinity and/or avidity for a binding partner, and/or prolonged biological half-life. The mimetic compounds of the disclosure can have a backbone that is partially or completely non-peptide, but with side groups identical to the side groups of the amino acid residues that occur in the peptide on which the mimetic compound is modeled. Several types of chemical bonds, for example, ester, thioester, thioamide, retroamide, reduced carbonyl, dimethylene and ketomethylene bonds, are known in the art to be generally useful substitutes for peptide bonds in the construction of protease-resistant mimetic compounds.

In one embodiment, peptides useful within the disclosure are modified to produce synthetic peptide mimetics by replacement of one or more naturally occurring side chains of the 20 genetically encoded amino acids (or D-amino acids) with other side chains, for example with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from a 5-membered ring to a 4-, 6-, or 7-membered ring. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and thiazolyl groups. These heterocyclic groups can be substituted or unsubstituted. Where a group is substituted, the substituent can be alkyl, alkoxy, halogen, oxygen, or substituted or unsubstituted phenyl. Peptides, as well as peptide analogs and mimetics, can also be covalently bound to one or more of a variety of nonproteinaceous polymers, for example, polyethylene glycol, polypropylene glycol, or polyoxyalkenes, as described in U.S. Pat. Nos. 4,640,835; 4,496,689; 4,301,144; 4,670,417; 4,791,192; and 4,179,337.

Other peptide analogs and mimetics within the scope of the disclosure include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues (e.g., lysine or arginine). Acyl groups are selected from the group of alkyl-moieties including C3 to C18 alkyl, thereby forming alkanoyl aroyl species. Also embraced are versions of a native primary amino acid sequence which have other minor modifications, including phosphorylated amino acid residues, for example, phosphotyrosine, phosphoserine, or phosphothreonine, or other moieties, including ribosyl groups or cross-linking reagents. In the peptides disclosed herein, the linkage between amino acid residues can be a peptide bond or amide linkage (e.g., —C—C(O)NH—). Alternatively, one or more amide linkages is optionally replaced with a linkage other than amide, for example, a substituted amide. Substituted amides generally include, but are not limited to, groups of the formula —C(O)NR—, where R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl, and substituted 6-26 membered alkheteroaryl. Additionally, one or more amide linkages can be replaced with peptidomimetic or amide mimetic moieties which do not significantly interfere with the structure or activity of the peptides. Suitable amide mimetic moieties are described, for example, in Olson et al., J. Med. Chem. 36:3039-3049, 1993.

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art.

### IV. Overview of Several Embodiments

Isolated peptides and peptide analogs with domains that promote lipid efflux from cells are disclosed herein. The isolated peptides and peptide analogs are believed to stimulate LCAT activity. In some embodiments, the isolated peptides and peptide analogs of the present invention contain domains that promote lipid efflux and also possess anti-inflammatory activity, for example the A and C domains in the ABC peptides or the X and Z domains in the XYZ peptides. Isolated peptides and peptide analogs that also include an additional functional domain or peptide are also disclosed herein. This additional functional domain provides anti-inflammatory biological activity, especially with regard to the domains indicated by W and/or D. This additional anti-inflammatory domain, or the domains that possess both lipid efflux and anti-inflammatory activity, provide additional benefit as many vascular conditions are considered by one of ordinary skill in the art to have inflammation as a component of the disease etiology.

For administration to an animal or a human, the peptides and peptide analogs of the present invention are combined with an acceptable carrier to form a pharmaceutical composition and are administered to the animal or the human.

In another embodiment, a method is provided for treating or inhibiting dyslipidemic and vascular disorders in an animal or a human. This method includes administering to the animal or the human a therapeutically effective amount of a pharmaceutical composition that includes one or more isolated peptides or peptide analogs and one or more anti-inflammatory domains. In specific, non-limiting examples, the dyslipidemic and vascular disorders include hyperlipidemia, hyperlipoproteinemia, hypercholesterolemia, hypertriglyceridemia, HDL deficiency, apoA-I deficiency, coronary artery disease, atherosclerosis, myocardial infarction, stroke, thrombotic stroke, peripheral vascular disease, restenosis, acute coronary syndrome, and reperfusion myocardial injury. In yet another specific example of the provided method, the isolated peptide includes two domains, one or more anti-inflammatory domains (D and W) and has an amino acid sequence as set forth herein. In yet another specific example of the provided method, the isolated peptide includes a domain or domains (A and C, X and Z) that possess both anti-inflammatory and lipid efflux activity and has an amino acid sequence as set forth herein.

Additionally, in representative peptides disclosed herein, the amino- and carboxy-terminal ends can be modified by conjugation with various functional groups. Neutralization of the terminal charge of synthetic peptide mimetics of apolipoproteins has been shown to increase their lipid affinity (Yancey et al., Biochem. 34:7955-7965, 1995; Venkatachalapathi et al., Protein: Structure, Function and Genetics 15:349-359, 1993). For example, acetylation of the amino terminal end of amphipathic peptides increases the lipid affinity of the peptide (Mishra et al., J. Biol. Chem. 269:7185-7191, 1994). Other possible end modifications are described, for example, in Brouillette et al., Biochem. Biophys. Acta 1256:103-129, 1995: Mishra et al., J. Biol. Chem. 269:7185-7191, 1994; and Mishra et al, J. Biol. Chem. 270:1602-1611, 1995.

In another embodiment, a detectable moiety can be linked to any of the peptides disclosed herein, creating a peptide-detectable moiety conjugate. The peptides or peptide analogs disclosed herein may be labeled using labels and techniques known to one of ordinary skill in the art. Some of these labels are described in the “Handbook of Fluorescent Probes and Research Products”, ninth edition, Richard P. Haugland (ed) Molecular Probes, Inc. Eugene, Oreg.), which is incorporated herein in its entirety. Detectable moieties suitable for such use include any composition detectable by spectroscopic, photochemical, biochemical, immunochemical, electrical, optical, magnetic or chemical means. The detectable moieties contemplated for the present disclosure can include, but are not limited to, an immunofluorescent moiety (e.g., fluorescein, rhodamine, Texas red, and the like), a radioactive moiety (e.g., ³H, ³²P, ¹²⁵I, ¹³¹I, ³⁵S), an enzyme moiety (e.g., horseradish peroxidase, alkaline phosphatase), a colorimetric moiety (e.g., colloidal gold, biotin, colored glass or plastic, and the like). The detectable moiety can be liked to the peptide or peptide analog at either the N- and/or C-terminus. Optionally, a linker can be included between the peptide or peptide analog and the detectable moiety.

The detectable peptides of the present invention may be employed in imaging techniques to identify sites of atherosclerotic plaque and sites of cholesterol efflux. Such imaging techniques may occur in vivo using IVUS, NMR, CAT, PET or other techniques commonly known to one of ordinary skill in the art.

Means of detecting such moieties are well known to those of skill in the art. Thus, for example, radiolabels may be detected using photographic film, gamma counters or scintillation counters. Fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The linkers contemplated by the present disclosure can be any bifunctional molecule capable of covalently linking two peptides to one another. Thus, suitable linkers are bifunctional molecules in which the functional groups are capable of being covalently attached to the N- and/or C-terminus of a peptide. Functional groups suitable for attachment to the N- or C-terminus of peptides are well known in the art, as are suitable chemistries for effecting such covalent bond formation.

The linker may be flexible, rigid or semi-rigid. Suitable linkers include, for example, amino acid residues such as Pro or Gly or peptide segments containing from about 2 to about 5, 10, 15, 20, or even more amino acids, bifunctional organic compounds such as H₂N(CH₂)ₙCOOH where n is an integer from 1 to 12, and the like. Examples of such linkers, as well as methods of making such linkers and peptides incorporating such linkers, are well-known in the art (see, e.g., Hunig et al., Chem. Ber. 100:3039-3044, 1974 and Basak et al., Bioconjug. Chem. 5:301-305, 1994).

Conjugation methods applicable to the present disclosure include, by way of non-limiting example, reductive amination, diazo coupling, thioether bond, disulfide-bond, amidation and thiocarbamoyl chemistries. In one embodiment, the amphipathic α-helical domains are “activated” prior to conjugation. Activation provides the necessary chemical groups for the conjugation reaction to occur. In one specific, non-limiting example, the activation step includes derivatization with adipic acid dihydrazide. In another specific, non-limiting example, the activation step includes derivatization with the N-hydroxysuccinimide ester of 3-(2-pyridyl dithio)-propionic acid. In yet another specific, non-limiting example, the activation step includes derivatization with succinimidyl 3-(bromoacetamido) propionate. Further, non-limiting examples of derivatizing agents include succinimidylformylbenzoate and succinimidyllevulinate.

### V. Synthesis and Purification of the Peptides

The peptides or peptide analogs of the disclosure can be prepared using virtually any technique known to one of ordinary skill in the art for the preparation of peptides. For example, the peptides can be prepared using step-wise solution or solid phase peptide syntheses, or recombinant DNA techniques, or the equivalents thereof

A. Chemical Synthesis

Peptides of the disclosure containing amino acids having either the D- or L-configuration can be readily synthesized by automated solid phase procedures well known in the art. Suitable syntheses can be performed by utilizing “T-boc” or “F-moc” procedures. Techniques and procedures for solid phase synthesis are described in Solid Phase Peptide Synthesis: A Practical Approach, by E. Atherton and R. C. Sheppard, published by IRL, Oxford University Press, 1989. Alternatively, the peptides may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). This is particularly the case with glycine containing peptides. Other methods useful for synthesizing the peptides of the disclosure are described in Nakagawa et al., J. Am. Chem. Soc. 107:7087-7092, 1985.

Additional exemplary techniques known to those of ordinary skill in the art of peptide and peptide analog synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups.

Peptides of the disclosure having either the D- or L-configuration can also be readily purchased from commercial suppliers of synthetic peptides. Such suppliers include, for example, Advanced ChemTech (Louisville, Ky.), Applied Biosystems (Foster City, Calif.), Anaspec (San Jose, Calif.), and Cell Essentials (Boston, Mass.).

B. Recombinant Synthesis

If the peptide is composed entirely of gene-encoded amino acids, or a portion of it is so composed, the peptide or the relevant portion can also be synthesized using conventional recombinant genetic engineering techniques. For recombinant production, a polynucleotide sequence encoding the peptide is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Sambrook et al. (ed.), Molecular Cloning: A Laboratory Manual, 2^{nd} ed., vol. 1-3, Cold Spring Harbor Laboratory Press, Cold Spring Harbor, N.Y., 1989, Ch. 17 and Ausubel et al. Short Protocols in Molecular Biology, 4^{th} ed., John Wiley & Sons, Inc., 1999).

To increase efficiency of production, the polynucleotide can be designed to encode multiple units of the peptide separated by enzymatic cleavage sites. The resulting polypeptide can be cleaved (e.g., by treatment with the appropriate enzyme) in order to recover the peptide units. This can increase the yield of peptides driven by a single promoter. In one embodiment, a polycistronic polynucleotide can be designed so that a single mRNA is transcribed which encodes multiple peptides, each coding region operatively linked to a cap-independent translation control sequence, for example, an internal ribosome entry site (IRES). When used in appropriate viral expression systems, the translation of each peptide encoded by the mRNA is directed internally in the transcript, for example, by the IRES. Thus, the polycistronic construct directs the transcription of a single, large polycistronic mRNA which, in turn, directs the translation of multiple, individual peptides. This approach eliminates the production and enzymatic processing of polyproteins and can significantly increase yield of peptide driven by a single promoter.

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage [symbol] , plac, ptrp, ptac (ptrp-lac hybrid promoter) and the like can be used. When cloning in insect cell systems, promoters such as the baculovirus polyhedron promoter can be used. When cloning in plant cell systems, promoters derived from the genome of plant cells (e.g., heat shock promoters, the promoter for the small subunit of RUBISCO, the promoter for the chlorophyll a/b binding protein) or from plant viruses (e.g., the 35S RNA promoter of CaMV, the coat protein promoter of TMV) can be used. When cloning in mammalian cell systems, promoters derived from the genome of mammalian cells (e.g., metallothionein promoter) or from mammalian viruses (e.g., the adenovirus late promoter, the vaccinia virus 7.5 K promoter) can be used.

C. Purification

The peptides or peptide analogs of the disclosure can be purified by many techniques well known in the art, such as reverse phase chromatography, high performance liquid chromatography, ion exchange chromatography, size exclusion chromatography, affinity chromatography, gel electrophoresis, and the like. The actual conditions used to purify a particular peptide or peptide analog will depend, in part, on synthesis strategy and on factors such as net charge, hydrophobicity, hydrophilicity, and the like, and will be apparent to those of ordinary skill in the art.

For affinity chromatography purification, any antibody which specifically binds the peptide or peptide analog may be used.

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art.

D. Antibody Production

For the production of antibodies, various host animals, including but not limited to, rabbits, mice, rats, and the like, may be immunized by injection with a peptide or peptide analog. The peptide or peptide analog can be attached to a suitable carrier (e.g., bovine serum albumin (BSA)) by means of a side chain functional group or linker attached to a side chain functional group. Various adjuvants may be used to increase the immunological response, depending on the host species, including but not limited to, Freund's (complete and incomplete), mineral gels (e.g., aluminum hydroxide), surface active substances (e.g., lysolecithin, pluronic polyols, polyanions, and oil emulsions), keyhole limpet hemocyanin, dinitrophenol, and potentially useful human adjuvants such as BCG (bacilli Calmette-Guerin) and Corynebacterium parvum.

Booster injections can be given at regular intervals, and antiserum harvested when the antibody titer thereof, as determined semi-quantitatively, for example, by double immunodiffusion in agar against known concentrations of the antigen, begins to fall. See, e.g., Ouchterlony et al., Handbook of Experimental Immunology, Wier, D. (ed.), Chapter 19, Blackwell, 1973. A plateau concentration of antibody is usually in the range of 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

Monoclonal antibodies to a peptide or peptide analog may be prepared using any technique which provides for the production of antibody molecules by continuous cell lines in culture, for example the classic method of Kohler & Milstein (Nature 256:495-97, 1975), or a derivative method thereof. Briefly, a mouse is repetitively inoculated with a few micrograms of the selected protein immunogen (e.g., a peptide or peptide analog) over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as enzyme-linked immunosorbent assay (ELISA), as originally described by Engvall (Meth. Enzymol., 70:419-39, 1980), or a derivative method thereof. Selected positive clones can be expanded and their monoclonal antibody product harvested for use. Detailed procedures for monoclonal antibody production are described in Harlow and Lane, Using Antibodies: A Laboratory Manual, CSHL, New York, 1999. Polyclonal antiserum containing antibodies can be prepared by immunizing suitable animals with a polypeptide comprising at least one peptide or peptide analog, which can be unmodified or modified, to enhance immunogenicity.

Antibody fragments may be used in place of whole antibodies and may be readily expressed in prokaryotic host cells. Methods of making and using immunologically effective portions of monoclonal antibodies, also referred to as “antibody fragments,” are well known and include those described in Better & Horowitz, Methods Enzymol. 178:476-96, 1989; Glockshuber et al., Biochemistry 29:1362-67, 1990; and U.S. Pat. Nos. 5,648,237 (Expression of Functional Antibody Fragments); 4,946,778 (Single Polypeptide Chain Binding Molecules); and 5,455,030 (Immunotherapy Using Single Chain Polypeptide Binding Molecules), and references cited therein. Conditions whereby a polypeptide/binding agent complex can form, as well as assays for the detection of the formation of a polypeptide/binding agent complex and quantitation of binding affinities of the binding agent and polypeptide, are standard in the art. Such assays can include, but are not limited to, Western blotting, immunoprecipitation, immunofluorescence, immunocytochemistry, immunohistochemistry, fluorescence activated cell sorting (FACS), fluorescence in situ hybridization (FISH), immunomagnetic assays, ELISA, ELISPOT (Coligan et al., Current Protocols in Immunology, Wiley, NY, 1995), agglutination assays, flocculation assays, cell panning, etc., as are well known to one of skill in the art.

E. Peptide Reconstitution

The peptides of the present invention may be reconstituted in any pharmaceutically acceptable carrier before use or administration. In one embodiment, the peptides may be reconstituted with saline, a lipid or a phospholipid, or a combination thereof. Some phospholipids that may be employed include but are not limited to the following: dipalmitoylphosphatidylcholine (DPPC); dioleoylphosphatidylcholine (DOPC); 1-palmitoyl-2-oleoylphosphatidylcholine (POPC); 1-palmitoyl-2-linoleoylphosphatidylcholine (PLPC); 1-palmitoyl-2-arachidonylphosphatidylcholine (PAPC); 1-palmitoyl-2-docosahexanoylphosphatidylcholine (PDPC); and, PMLC. DPPC, DOPC have been used to reconstitute peptides (Shah et al., Circulation. 2001 Jun. 26; 103(25):3047-50.)

The peptides of the present invention may be complexed with lipids or phospholipids in weight ratios ranging from 1:0.5 to 1:10, or 1:1 to 1:5. Any ratio within these ranges may be employed.

The phospholipids may also be complexed with other agents, such as sphingomyelin before complexing with the peptides of the present invention. Ratios of phospholipids to sphingomyelin include ratios occurring in the ranges of 1:9 to 9:1, 1:5 to 5:1, 1.2 to 2.1 (all weight %).

The peptides of the present invention may be complexed with the combination of phospholipid:sphingomyelin in weight ratios ranging from 1:0.5 to 1:10, or 1:1 to 1:5. Any ratio within these ranges may be employed.

### VI. Pharmaceutical Compositions and Uses Thereof

The peptides or peptide analogs of the disclosure can be used, alone or in combination, together with a pharmaceutically acceptable carrier, to treat any disorder in animals, especially mammals (e.g., humans), for which promoting lipid efflux and/or decreasing inflammation is beneficial. Such conditions include, but are not limited to, hyperlipidemia (e.g., hypercholesterolemia), cardiovascular disease (e.g., atherosclerosis), cerebrovascular disease, restenosis (e.g., atherosclerotic plaques), peripheral vascular disease, acute coronary syndrome, reperfusion myocardial injury, and the like. The peptides or peptide analogs of the disclosure can also be used alone or in combination during the treatment of thrombotic stroke, infarcts secondary to occlusion of a vessel and during thrombolytic treatment of occluded coronary artery disease. The peptides or peptide analogs of the disclosure can be used to treat tissue following hypoxia, ischemia and infarction due to impairment of blood supply, and also following hemorrhage following rupture or trauma of a blood vessel. Such tissue includes, without limitation, neural tissue in the central or peripheral nervous system, peripheral vascular tissue, and cardiac muscle.

It is to be understood that a mixture of peptides may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity. In one exemplary embodiment, one or more of the peptides shown in SEQ ID NOs: 121, 130, 155, 618, 624, may be combined in a mixture for administration.

The peptides or peptide analogs can be used alone or in combination therapy with other lipid lowering compositions or drugs and/or other anti-inflammatory compositions or drugs used to treat the foregoing conditions. Such therapies include, but are not limited to simultaneous or sequential administration of the drugs involved. For example, in the treatment of hypercholesterolemia or atherosclerosis, the peptide or peptide analog formulations can be administered with any one or more of the cholesterol lowering therapies currently in use, for example, bile-acid resins, niacin, statins, fat uptake inhibitors, and HDL raising drugs.

In another embodiment, the peptides or peptide analogs can be used in conjunction with statins or fibrates to treat hyperlipidemia, hypercholesterolemia and/or cardiovascular disease, such as atherosclerosis. In yet another embodiment, the peptides or peptide analogs of the disclosure can be used in combination with an anti-microbial agent and/or an anti-inflammatory agent, such as aspirin. In another embodiment peptides or peptide analogs of the disclosure can be used in combination with anti-hypertensive medicines known to one of ordinary skill in the art. It is to be understood that more than one additional therapy may be combined with administration of the peptides or peptide analogs of the disclosure.

In a further embodiment, the peptides can also be expressed in vivo, by using any of the available gene therapy approaches.

In yet another embodiment, the peptides or peptide analogs can be used in conjunction with medicines used to treat patients with cerebrovascular and cardiovascular disease resulting in hypoxia, ischemia and infarction due to impairment of blood supply, and also following hemorrhage following rupture or trauma of a blood vessel. Such medicines are commonly known to one of ordinary skill in the art and include without limitation, modulators of excitatory amino acids and modulators of platelet aggregation.

A. Administration of Peptides or Peptide Analogs

In some embodiments, peptides or peptide analogs can be isolated from various sources and administered directly to the animal or human. For example, a peptide or peptide analog can be expressed in vitro, such as in an E. coli expression system, as is well known in the art, and isolated in amounts useful for therapeutic compositions. The peptide or peptide analogs of the present invention may also be made though peptide synthetic methods known to one of ordinary skill in the art, such as solid phase synthesis.

In exemplary applications, therapeutic compositions comprising the peptide or peptide analogs in an acceptable carrier are administered to an animal or a human suffering from a dyslipidemic or vascular disorder, such as hyperlipidemia, hyperlipoproteinemia, hypercholesterolemia, hypertriglyceridemia, HDL deficiency, apoA-I deficiency, coronary artery disease, atherosclerosis, stroke, ischemia, infarction, myocardial infarction, hemorrhage, peripheral vascular disease, restenosis, acute coronary syndrome, or reperfusion myocardial injury, in an amount sufficient to inhibit or treat the dyslipidemic or vascular disorder. Amounts effective for this use will depend upon the severity of the disorder and the general state of the subject's health. A therapeutically effective amount of the compound is that which provides either subjective relief of a symptom(s) or an objectively identifiable improvement as noted by the clinician or other qualified observer.

A peptide or peptide analog can be administered by any means known to one of skill in the art (see, e.g., Banga, “Parenteral Controlled Delivery of Therapeutic Peptides and Proteins,” in Therapeutic Peptides and Proteins, Technomic Publishing Co., Inc., Lancaster, Pa., 1995), such as by intramuscular, subcutaneous, or intravenous injection, but even oral, nasal, or anal administration is contemplated. In one embodiment, administration is by subcutaneous or intramuscular injection. To extend the time during which the peptide or peptide analog is available to inhibit or treat a dyslipidemic or vascular disorder, the peptide or peptide analog can be provided as an implant, an oily injection, or as a particulate system. The particulate system can be a microparticle, a microcapsule, a microsphere, a nanoparticle, or similar particle (Banga, “Parenteral Controlled Delivery of Therapeutic Peptides and Proteins,” in Therapeutic Peptides and Proteins, Technomic Publishing Co., Inc., Lancaster, Pa., 1995). The peptide or peptide analog may also be applied to a medical device for delivery to a specific location. For example, a surgical tool, catheter, stent, balloon, electrode, suture, or an artificial vessel or transplanted vessel may contain or be coated with the peptide or peptide analog.

It is to be understood that in some embodiments, one or more of the amino acids of the peptides of the present invention are D amino acids. In one embodiment, the N-terminal amino acid, the C-terminal amino acid or both are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

In one specific, non-limiting example, a peptide is administered that includes one or more of the amino acid sequences disclosed herein.

B. Representative Methods of Administration, Formulations and Dosage

The provided peptides or peptide analogs, constructs, or vectors encoding such peptides, can be combined with a pharmaceutically acceptable carrier (e.g., a phospholipid or other type of lipid) or vehicle for administration to human or animal subjects. As described previously in the application, the peptides may be reconstituted with acceptable carriers such as saline, lipid, phospholipid, lipid:sphingomyelin complexes and phospholipid: sphingomyelin complexes. In some embodiments, more than one peptide or peptide analog can be combined to form a single preparation. The peptides or peptide analogs can be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of a sterile liquid carrier, for example, water for injections, immediately prior to use. Extemporaneous injection solutions and suspensions may be prepared from sterile powders, granules and tablets commonly used by one of ordinary skill in the art.

In certain embodiments, unit dosage formulations are those containing a dose or unit, or an appropriate fraction thereof, of the administered ingredient. It should be understood that in addition to the ingredients particularly mentioned above, formulations encompassed herein may include other agents commonly used by one of ordinary skill in the art.

The pharmaceutical compositions provided herein, including those for use in treating dyslipidemic and vascular disorders, may be administered through different routes, such as oral, including buccal and sublingual, rectal, parenteral, aerosol, nasal, intramuscular, intraperitoneal, intravascular, subcutaneous, intradermal, and topical. They may be administered in different forms, including but not limited to solutions, emulsions and suspensions, microspheres, particles, microparticles, nanoparticles, and liposomes. In one embodiment, peptides or peptide analogs with suitable features of lipid efflux and low cytotoxicity can be precomplexed with phospholipids or other lipids into either discoidal or spherical shape particles prior to administration to subjects.

In another embodiment, it may be desirable to administer the pharmaceutical compositions locally to the area in need of treatment. This maybe achieved by, for example, and not by way of limitation, local or regional infusion or perfusion during surgery, direct perfusion into a vessel, such as an atherosclerotic vessel, topical application (e.g., wound dressing, peptide coated stent), injection, catheter, suppository, or implant (e.g., implants formed from porous, non-porous, or gelatinous materials, including membranes, such as silastic membranes or fibers), and the like. In one embodiment, administration can be by direct injection at the site (or former site) of a tissue that is to be treated, such as the heart or the peripheral vasculature. In another embodiment, the pharmaceutical compositions are delivered in a vesicle, in particular liposomes (see, e.g., Langer, Science 249:1527-1533, 1990; Treat et al., in Liposomes in the Therapy of Infectious Disease and Cancer, Lopez-Berestein and Fidler (eds.), Liss, N.Y., pp. 353-365, 1989). Combinations of administration methods may also be employed such as a systemic or local infusion of a peptide of the present invention, before, after or during placement of a stent coated with a peptide of the present invention. In yet another embodiment, the pharmaceutical compositions can be delivered in a controlled release system. In one embodiment, a pump can be used (see, e.g., Langer Science 249:1527-1533, 1990; Sefton Crit. Rev. Biomed. Eng. 14:201-240, 1987; Buchwald et al., Surgery 88:507-516, 1980; Saudek et al., N. Engl. J. Med. 321:574-579, 1989). In another embodiment, polymeric materials can be used (see, e.g., Ranger et al., Macromol. Sci. Rev. Macromol. Chem. 23:61-64, 1983; Levy et al., Science 228:190-192, 1985; During et al., Ann. Neurol. 25:351-356, 1989; and Howard et al., J. Neurosurg. 71:105-112, 1989). Other controlled release systems, such as those discussed in the review by Langer (Science 249:1527-1533, 1990), can also be used.

The amount of the pharmaceutical compositions that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each subject's circumstances. An example of such a dosage range is 0.1 to 200 mg/kg body weight in single or divided doses. Another example of a dosage range is 1.0 to 100 mg/kg body weight in single or divided doses.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the subject undergoing therapy.

The pharmaceutical compositions of the present disclosure can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. The volume of administration will vary depending on the route of administration. By way of example, intramuscular injections may range from about 0.1 ml to about 1.0 ml. Those of ordinary skill in the art will know appropriate volumes for different routes of administration.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

The subject matter of the present disclosure is further illustrated by the following non-limiting Examples.

### Example 1

### Lipid Efflux from Cells Mediated by Peptides of the Present Invention

This example demonstrates a method to test the ability of peptides of the present invention to efflux lipid from ABCAl-expressing cells.

HeLa cells stably transfected with human ABCAl cDNA (ABCAl cells) and HeLa cells transfected with only a hygromycin-resistant control plasmid (control cells) are produced and grown in a-modified Eagle's medium (aMEM) plus 10% fetal calf serum, as described by Remaley et al. (Biochem. Biophys. Res. Commun. 280:818-823, 2001). Cholesterol and phospholipid efflux is performed for 18 hours on noncholesterol-loaded cells radiolabeled with either cholesterol or choline (Remaley et al., Arterioscler. Thromb. Vasc. Biol. 17:1813-1821, 1997). Percentage efflux is calculated after subtracting the radioactive counts in the blank media (aMEM plus 1 mg/ml of BSA), and expressed as the percent of total radioactive counts removed from the cells during the efflux period.

Cell fixation is performed by a 10 minute treatment with 3% paraformaldehyde in phosphate buffered saline (PBS), followed by three washes with blank media. Lactate dehydrogenase (LDH) release from cells into the media is measured enzymatically (Roche Diagnostics, Indianapolis, Ind.) and expressed, after subtraction of LDH released into blank media, as the percentage of total cell LDH. Total cell LDH is determined after cell solubilization with 1% Triton X-100.

The peptides of the present invention are synthesized by a solid-phase procedure, using a Fmoc/DIC/HOBt protocol on a Biosearch 9600 peptide synthesizer (Applied Biosystems, Foster City, Calif.), or an equivalent instrument. Both L-amino acid and D-amino acid enantiomers are synthesized. All peptides are purified to greater than 98% homogeneity by reverse-phase HPLC on an Aquapore RP-300 column, or similar chromatographic procedure.

ABCAl cells are used to assess the ability of apoA-I and synthetic peptides to efflux lipid from cells. As previously described (Hamon et al., Nat. Cell Biol. 2:399-406, 2000 and Remaley et al., Biochem. Biophys. Res. Commun. 280:818-823, 2001), control cells do not efflux significant amounts of cholesterol and phospholipid to apoA-I, but do so after transfection with ABCAl. The peptides of the present invention efflux approximately 2- to 4-fold more cholesterol and phospholipid from ABCAl cells than from control cells. Both the peptides of the present invention and apoA-I began to show saturation for lipid efflux at approximately the same protein concentration of 10 μg/ml.

The peptides of the present invention remove more cholesterol and phospholipids from control cells than apoA-I.

### Example 2

### Lipid Efflux Time Course

This example demonstrates the cholesterol efflux time course from ABCAl-expressing cells to apoA-I and peptides of the present invention.

Cholesterol efflux from ABCAl cells to apoA-I is first detectable after 2 hours and increases throughout the 30 hour efflux period. In contrast, there is no significant increase above background in cholesterol efflux to apoA-1 from control cells. Overall, the kinetics for cholesterol efflux to peptides of the present invention from ABCAl cells is similar to that of apoA-1, except that cholesterol efflux is first detectable after 30 minutes. The peptides of the present invention, unlike apoA-1, also promote cholesterol efflux from control cells but at a lower rate.

### Example 3

### Identification of Non-Cytotoxicpeptides that Promote ABCAl-Dependent Lipid Efflux

This example illustrates a method for identifying non-cytotoxic peptides that promote ABCAl-dependent lipid efflux from cells.

Peptide Design Based on the principles and procedures described in the present application, an amino acid sequence can be designed for a peptide that promotes lipid efflux.

Peptide production: Peptides to be tested can be produced synthetically or by recombinant DNA methods, as described in the present application, and purified by reverse phase HPLC or other suitable techniques well known to one of skill in the art.

Peptide Cytotoxicity Testing: Peptides can be tested for cytotoxicity by any number of methods well known to one of skill in the art, such as the release of intracellular LDH.

Peptide ABCAl-specificity for Lipid Efflux: Peptides to be tested can be added to serum-free cell culture media in the approximate concentration range of 1-20 micrograms and incubated with a control cell line that does not express the ABCAl transporter and the same cell line after transfection with human cDNA for the ABCAl transporter, as described herein. Alternatively, cells, such as macrophages, that either express or do not express the ABCAl transporter depending on their cholesterol content and/or exposure to agents that induce the ABCAl transporter (e.g., cAMP and LXR agonists) can also be used. After a suitable period of approximately 4 to 24 hours, the conditioned media can be removed from the cells and the amount of cholesterol and or phospholipid effluxed can be quantified, as described herein. ABCAl-specific lipid efflux is calculated by subtracting the total lipid efflux of the cell line that does not express the ABCAl transporter from the lipid efflux from the ABCAl expressing cell line.

### Example 4

### Peptides of the Present Invention Reduce Atherosclerosis in Animal Models

The ability of the peptides of the present invention and associated fragments are tested in apoE knockout mice on a chow diet and LDL receptor knockout mice on a western high fat diet to determine the effect of these peptides to reduce atherosclerosis in a mouse model system. One or more of the peptides of the present invention, in a range of concentration of 2 mg/kg to 50 mg/kg, is injected intravenously (iv) or intraperitoneally (ip) 2 to 3 times per week over a period of approximately 6 weeks. Aortic atherosclerosis is quantitated in the aortic arch before administration of the peptides and after the 6 week period of administration. (Wu et al., J. Biol. Chem.; 2004: 279, 22913-22925). The results demonstrate reduced atherosclerosis in the aortic arch in mice in both treatment groups.

### Example 5

### Administration of the Peptides of the Present Invention to Treat Atherosclerosis in Humans

Individuals with acute coronary syndrome and documented atherosclerosis have a cardiac catherization with intravascular ultrasound (IVUS) to document coronary atherosclerosis of 20 to 50% obstruction in the target artery. Each individual is on stable hypolipidemic drug therapy and receives an acceptable dose of a peptide of the present invention and/or an associated fragment iv weekly for a period of 5 to 8 weeks. A repeat IVUS measurement is made at the end of the treatment period to assess the effect of the peptide infusion on coronary atherosclerosis in the target vessel. Plaque is reduced in the atherosclerotic coronary artery following the peptide treatment demonstrating efficacy of the peptides of the present invention to treat atherosclerosis.

### Example 6

### Administration of the Peptides of the Present Invention to Prevent or Delay the Onset of Atherosclerosis in Humans

Individuals with documented risk factors for atherosclerosis and having high plasma cholesterol levels have a ultrasound analysis of the coronary (IVUS), carotid (IMT) or popliteal arteries to establish a baseline measurement. A portion of these individuals are daily administered individual peptides of the present invention at a dose of 2 mg/kg to 50 mg/kg intravenously (iv) or intramuscular (im) 1 to 3 times per week over a period of approximately one to six months. The other individuals receive a control peptide. A new ultrasound analysis at the end of the treatment period indicates higher levels of plaque in the vessels of individuals receiving the control peptide. This example indicates that the individual peptides of the present invention are effective in preventing or reducing atherosclerosis in individuals at risk for developing atherosclerosis and in reducing plaque accumulation in coronary, carotid or popliteal arteries.

### Example 7

### Administration of the Peptides of the Present Invention on Stents to Reduce Inflammation and Restenosis

Individuals with acute coronary syndrome and having plaque in coronary vessels which require a stent to reduce the obstruction receive an IVUS procedure to document the coronary anatomy. A representative protocol divides these individuals into three groups. One group receives a stent coated with a peptide of the present invention. A second group receives an iv infusion of a peptide of the present invention at a dose of 2 mg/kg to 50 mg/kg, 1 to 3 times per week over a period of approximately 5 to 10 weeks. A third group receives a stent coated with a peptide of the present invention and an iv infusion of a peptide of the present invention at a dose of 2 mg/kg to 50 mg/kg, 1 to 3 times per week over a period of approximately 5 to 10 weeks.

All individuals receive a second IVUS procedure at the end of 5 or 10 weeks. The results demonstrate that individuals receiving either a peptide coated stent, a peptide coated stent plus iv peptide infusion, or iv peptide infusion alone, all display reduced inflammation and restenosis when compared to their condition at the time of the first IVUS procedure.

### Example 8

### Blockade of ICAM-1/LFA-1 Mediated T-Cell Adhesion to Caco-2 Cell Monolayers by the Peptides of the Present Invention

The ability of the peptides of the present invention and associated fragments are tested to decrease inflammation by their ability to block the binding of ICAM-1 to LFA-1 using a model cell adhesion assay of T cells (Mott-3) and Caco-2 cells (Anderson et al., Bioorganic & Medicinal Chemistry Letters; 2004:14, 1399-1402). Peptide concentrations of from 0 μM to 500 μM are tested. The results demonstrate dose dependent inhibition of ICAM-1/LFA-1 mediated T-cell adhesion to Caco-2 cell monolayers by the peptides of the present invention. While not wanting to be bound by the following statement, it is believed that the D or D′ domains of the peptides of the present invention are involved in this inhibitory effect. While not wanting to be bound by the following statement, it is believed in other embodiments that the A or C, or the X and Z, domains of some of the peptides of the present invention are involved in this inhibitory effect.

These results indicate that the interaction of ICAM-1 and LFA-1 in the vessel wall can be blocked by the D domain of the peptides of the present invention, and result in decreased movement of inflammatory cells, particularly T cells, from the plasma into the vessel wall. A decrease in the influx of inflammatory cells into the vessel wall decreases this inflammatory component of the atherosclerotic process and decreases the frequency of clinical vascular events (Yusuf-Makagiqansar, Inflammation: 2001; 25, 203-213).

### Example 9

### Blockade of Neutrophils through Inhibition of the Formyl Peptide Receptor-Like-1 (FPRL1) by the Peptides of the Present Invention

The anti-inflammatory properties of the peptides of the present invention and associated fragments are tested by evaluating the peptides, and particularly the W and W″ domains, and peptides containing these domains, to block the binding of neutrophils to the formyl peptide-like 1 receptor using techniques as described by Bae et al., (Bae et al Journal of Immunology; 2004: 173, 607-614; Bae et al., Journal of Immunology; 2003: 171, 6807-6813). The peptides of the present invention are tested in a range of 1 pM to 10 μM for their ability to inhibit the binding of radiolabelled SEQ ID NO: 629 Trp Lys Tyr Met Val MET peptide to FPRL1 expressing RBL-2H3 cells, and for their ability to block SEQ ID NO: 629 Trp Lys Tyr Met Val MET induced cellular chemotaxis in FPRL1 expressing RBL-2H3 cells. The peptides of the present invention are also tested in other assays described in these two references by Bae et al.

The results demonstrate that the anti-inflammatory properties of the peptides of the present invention and associated fragments, including peptides containing the W domain, inhibit the binding of radiolabelled SEQ ID NO: 629 Trp Lys Tyr Met Val MET peptide to FPRL1 expressing RBL-2H3 cells, inhibit SEQ ID NO: 629 Trp Lys Tyr Met Val MET induced cellular chemotaxis in FPRL1 expressing RBL-2H3 cells and decrease superoxide generation.

While not wanting to be bound by the following statement, it is believed that administration of the peptides of the present invention to individuals decreases the early neutrophil influx into the vessel wall mediated by the formyl peptide-like 1 receptor in acute myocardial infarction or acute coronary syndrome resulting in a decrease in the inflammatory component of atherosclerosis, thereby reducing subsequent clinical events and post-perfusion injury.

### Example 10

### Use of Labelled Peptides of the Present Invention to Visualize and Locate Plaque in Atherosclerotic Vessels

The peptides of the present invention are complexed with phospholipids as well as gadolinium or other suitable reagent and the recombined particle is targeted to cholesterol filled cells which have increased expression of the ABCAl transporter in the vulnerable plaque of the coronary artery. It is believed that the peptides of the present invention have a high affinity for the ABCAl transporter and are anticipated to bind to only those cells with an increased intracellular level of cholesterol which induced upregulation of the ABCAl transporter.

These studies on the peptides of the present invention and associated fragments are compared to results from studies employing ApoA-I protein/phospholipid complex to determine the specificity and selectivity of the peptides of the present invention versus ApoA-I in the localization of the label to vulnerable plaque. The use of the labeled peptides of the present invention to visualize vulnerable plaque provides a valuable tool for diagnosis and treatment of patients at risk for developing cardiovascular disease. (Frias et al., J Am Chem Soc; 2004:126, 16316-7).

### Example 11

### Synthesis of SEQ ID NO: 121 Ser Pro Leu Ser Asp Glu Leu Arg Gln Arg Leu Ala Ala Arg Leu Glu Ala Leu Lys Glu Asn Lys Leu Ser Pro Leu Leu Glu Ser Phe Lys Val Ser Phe Leu Ser Ala Leu Glu Glu Tyr Thr Lys Lys Leu Asn Thr Gln

The peptide was synthesized manually on Fmoc-Gln(Trt) PEG resin via Fmoc chemistry. Protecting groups used for amino acids were: t-Butyl group for Ser, Thr, Asp, Glu and Tyr, Trt group for Asn and Gln, Boc group for Lys, Pbf for Arg. Fmoc protected amino acids were purchased from EMD Biosciences. Reagents for coupling and cleavage were purchased from Aldrich. Solvents were purchased from Fisher Scientific. The peptide chain was assembled on resin by repetitive removal of the Fmoc protecting group and coupling of protected amino acid. DIC and HOBt were used as coupling reagent and NMM was used as base. 20% piperidine in DMF was used as de-Fmoc-reagent. After removal of last Fmoc protecting group, resin was treated with cocktail K for cleavage and removal of the side chain protecting groups.

Crude peptide was precipitated from cold ether and collected by filtration. Purification of crude peptide was achieved via RP-HPLC by using polymer column from Polymer Laboratories. Peptide was purified using TFA Buffer. Pooled fractions were lyophilized. The peptide was verified by MS analysis and amino acid analysis. The peptide purity was determined by analytical HPLC column (Phenomenex, JupiterC18, 4.6×250 mm, 5 micron).

### Example 12

### Synthesis of SEQ ID NO: 624 Ac-Ser Pro Leu Leu Glu Ser Ala Lys Val Ser Ala Leu Ser Ala Leu Glu Glu Ala Thr Lys Lys Lys Leu Ser Pro Leu Leu Glu Ser Phe Lys Val Ser Phe Leu Ser Ala Leu Glu Glu Tyr Thr Lys Lys Leu Asn Thr Gln-NH₂

The peptide was synthesized manually on Fmoc-Rink Amide PEG resin via Fmoc chemistry. Protecting groups used for amino acids were: t-Butyl group for Ser, Thr, Glu and Tyr, Trt group for Asn and Gln, Boc group for Lys. Fmoc protected amino acids were purchased from EMD Biosciences. Reagents for coupling and cleavage were purchased from Aldrich. Solvents were purchased from Fisher Scientific. The peptide chain was assembled on resin by repetitive removal of the Fmoc protecting group and coupling of protected amino acid. HBTU and HOBt were used as coupling reagent and NMM was used as base. 20% piperidine in DMF was used as de-Fmoc-reagent. After removal of last Fmoc protecting group, resin was treated with TFA/TIS/H₂O (95:3:2) for cleavage and removal of the side chain protecting groups.

Crude peptide was precipitated from cold ether and collected by filtration. Purification of crude peptide was achieved via RP-HPLC using 47 mm×300 mm column from Waters. Peptide was purified using TFA Buffer. Pooled fractions were lyophilized. The peptide has been verified by MS analysis and amino acid analysis. The peptide purity was determined by analytical HPLC column (Supelco C18, 4.6×250 mm).

### Example 13

### Analysis of SR-B1 Mediated Efflux and ABCAl Mediated Efflux

The methods employed in this study have been described in U.S. Pat. Nos. 7,029,863, 7,060,452, U.S. Patent Application Publication No. 2005/0191715, and in Moya et al., Arteriosclerosis & Thrombosis 1994:14:1056-1065 and Liu et al., J. Biol. Chem., 2003:278(44), 42976-42984. SR-B1 mediated cholesterol efflux was examined in FU5AH rat hepatoma cells and ABCAl mediated cholesterol efflux was examined in J774 mouse macrophage cells as described in these references.

TABLE 1
A. Efflux Assay with Peptides minus blank

| | SR-BI Mediated | ABCA1 Mediated | | |
|---|---|---|---|---|
| | Efflux | Efflux | +ABCA1 | −ABCA1 |
| Peptides* | % Per 4 h | % Per 4 h | Cells | Cells |
| 1 | 0.15 | 18.71 | 20.338 ± | 1.624 ± |
| | | | 0.136 | 0.126 |
| 2 | 0.16 | 13.08 | 14.283 ± | 1.205 ± |
| | | | 0.545 | 0.211 |
| 3 | 0.16 | 12.14 | 13.447 ± | 1.306 ± |
| | | | 0.549 | 0.261 |
| 4 | 0.10 | 11.20 | 12.420 ± | 1.224 ± |
| | | | 1.019 | 0.121 |
| 5 | 0.25 | 14.50 | 15.718 ± | 1.215 ± |
| | | | 0.123 | 0.288 |

Legend: 1 = SEQ ID NO: 624, 2 = SEQ ID NO: 121, 3 = SEQ ID NO: 121, 4 = SEQ ID NO: 130, 5 = SEQ ID NO: 624

TABLE 2
B. Controls for Efflux Assay with Samples minus blank

| | SR-BI | ABCA1 | | |
|---|---|---|---|---|
| | Mediated | Mediated | | |
| | Efflux | Efflux | +ABCA1 | −ABCA1 |
| Controls | % Per 4 h | % Per 4 h | Cells | Cells |
| 2% Human | 8.47 | 12.56 | 24.658 ± | 12.100 ± |
| Serum Pool | | | 0.130 | 0.485 |
| Apo A-I | 0.26 | 18.97 | 20.682 ± | 1.713 ± |
| @ 20 μg/ml | | | 0.724 | 0.409 |

* Efflux for all peptide samples was run at 30 μg/ml.

The results demonstrate that peptides SEQ ID NO: 121, SEQ ID NO: 130, and the N-terminally acetylated and C-terminally amidated form of LSI 130 which is SEQ ID NO:624, each stimulated efflux of cholesterol from J774 macrophage cells (ABCAl pathway) while having negligible or no effect on cholesterol efflux from the Fu5AH cells (SRB1 pathway), similar to the effect of Apo AI. These selective effects of these peptides demonstrates their efficacy to act as ApoA-I mimetics and selectively efflux cholesterol from cells.

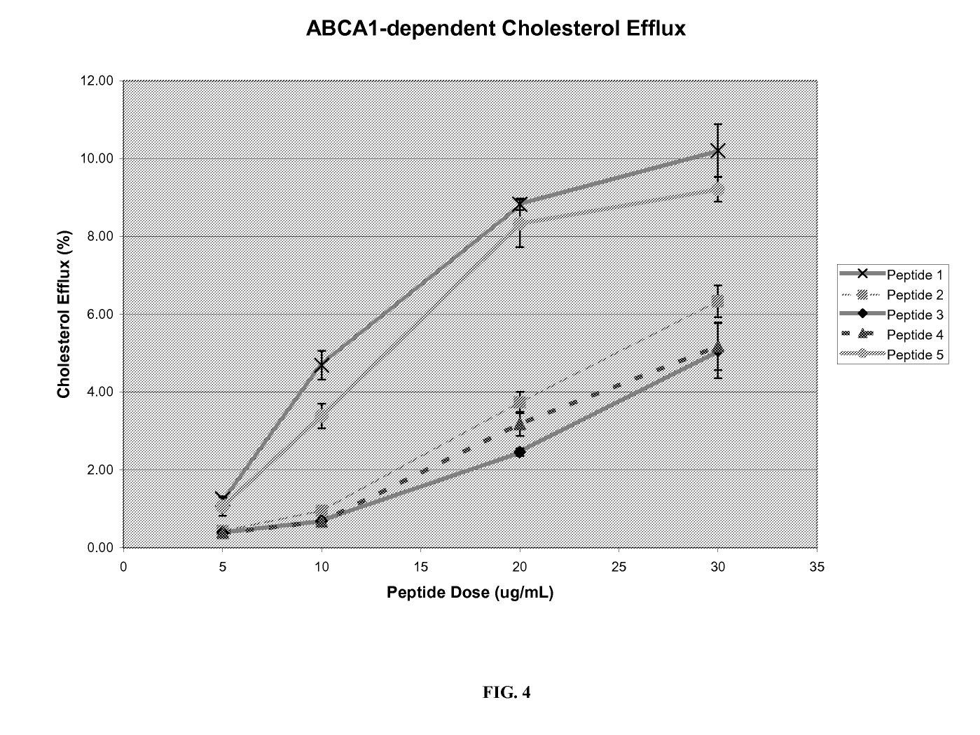

These effects were also dose dependent as shown in FIG. 4 with increasing efflux activity demonstrated through the range of 5 ug/ml to 30 ug/ml. Based on these in vitro efflux studies, the elevation of the Apo AI mimetic peptides of the present invention, in plasma, is expected to decrease coronary and other forms of atherosclerosis in high risk patients.

### Example 14

### Effect of the Peptides of the Present Invention on CD11b Expression in Monocytes Methods

Monocyte Isolation Peripheral whole blood (PWB) was drawn from healthy consenting individuals into syringes containing sodium citrate (final concentration—19.2 mM). Resting human monocytes were isolated from PWB by density centrifugation with Lymphoprep (Axis Shield). Mononuclear cells (MNCs) were collected and monocytes were further separated to purity using the Dynal negative isolation kit (Invitrogen). Monocytes were resuspended in phosphate buffered saline (PBS) and cell number was determined counting cell suspension on an automated hematology analyzer (Sysmex, KX-21N, USA). Purification of HDL and apoA-1 Human plasma apoA-1 was isolated as previously described and the purity determined using total mass spectrometry.

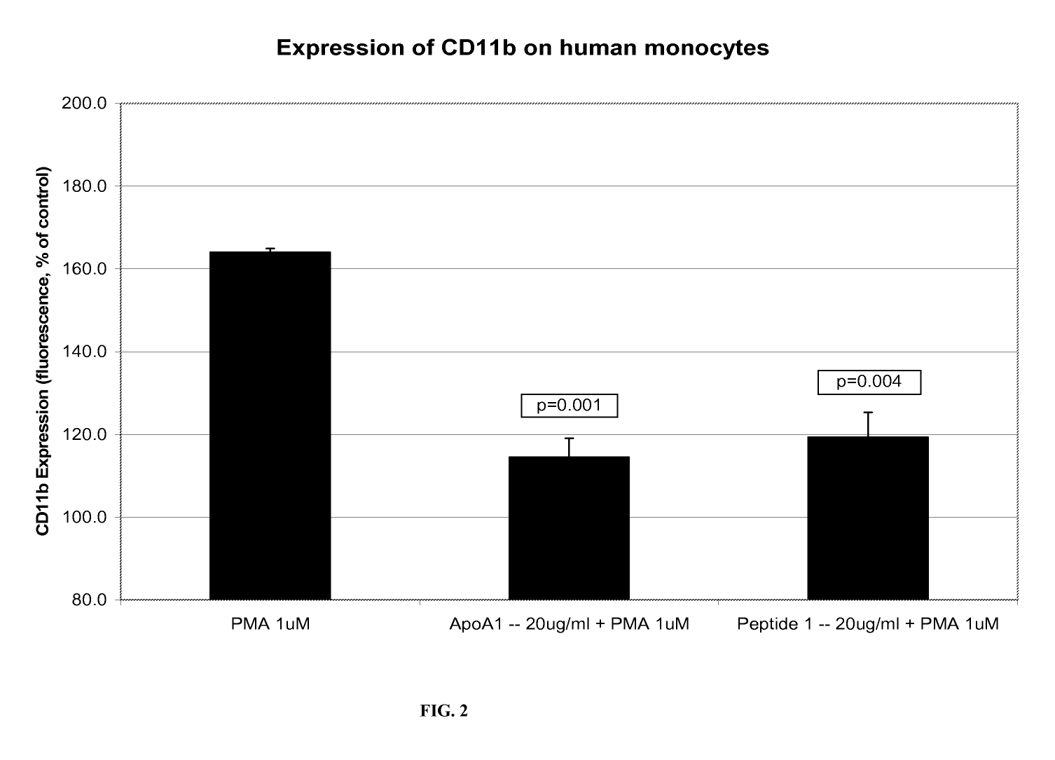

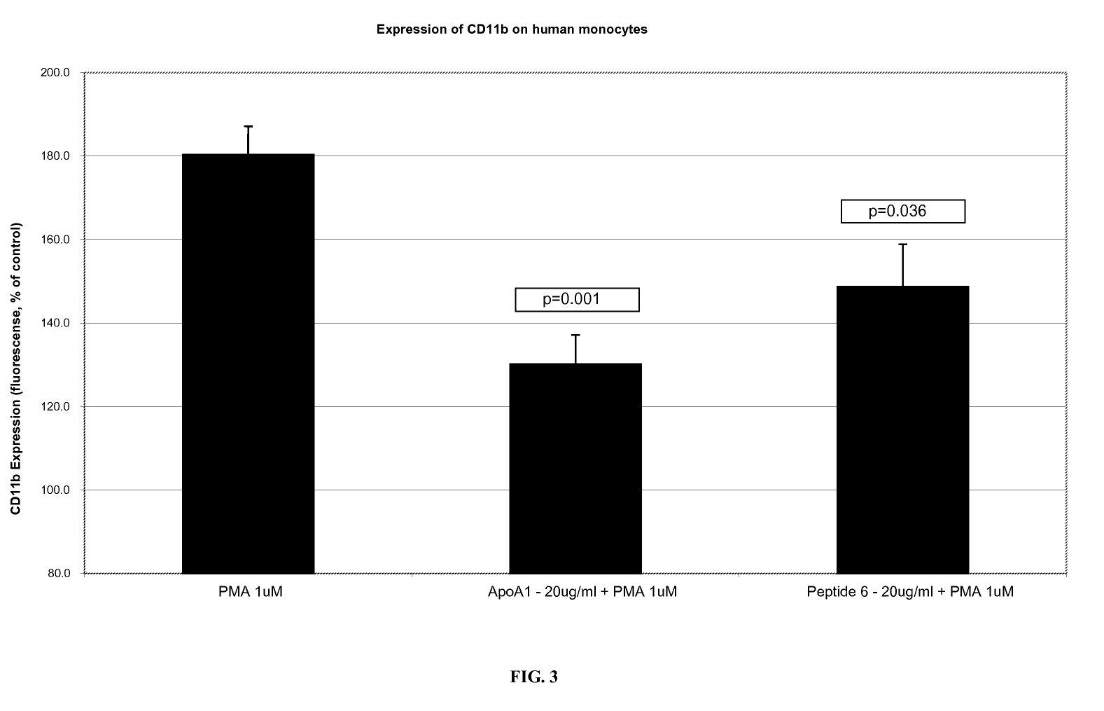

Flow Cytometry 100 μL of monocytes were stimulated with either 1 μmol/L phorbol-myristate-acetate (PMA) or 1 μg/ml lipopolysaccharide (LPS) (Sigma, Australia) in the presence or absence of apoA-1 (20 μg/ml), or 20 μg/ml of the test peptides SEQ ID NOs: 155, 624, each tested separately. The cells were incubated with the FITC conjugated antibody to either the active epitope of CD11b (eBiosciences, USA, Clone CBRM1/5) or total CD11b (Serotec, USA, Clone ICRF44) for 15 min at 37° C. Cells were then fixed with 4% para-formaldehyde. Samples were controlled for by using the appropriately matched isotype matched negative control (FITC-anti-mouse IgG) (Serotec, USA, Clone W3/25). CD11b expression was measured by flow cytometry using FACS Calibur (Becton Dickinson). Analysis was conducted using the Cell Quest Pro software.
Statistical Analysis Values are presented as the mean ±SD or percentage of control ±SD. FACS results were analyzed for statistical significance using one-way ANOVA followed by Bonferroni post-hoc test. Significance was accepted at P<0.05.
Results As expected, ApoA1 (SEQ ID NO: 36) significantly reduced PMA induces CD11b expression. SEQ ID NO: 624 significantly reduced PMA induced CD11b expression (FIG. 2). SEQ ID NO: 155 significantly reduced the inflammatory response measured as a reduction in CD11b expression (FIG. 3). These results demonstrate that SEQ ID NO: 624 and SEQ ID NO: 155, significantly inhibited PMA induced CD11b expression. Taken together, the results demonstrate the anti-inflammatory properties of these peptides of the present invention. The combined effects of increasing cholesterol efflux and decreasing inflammation indicate that the peptides of the present invention effectively mimic the function of Apo AI and will decrease atherosclerosis.

### Example 15

### Evaluation of Peptide Utility in ApoE Knockout and LDL Receptor Knockout Mice

ApoE knockout and LDL receptor knockout mice, two well established animal models for the study of atherosclerosis, are injected with either saline as control or the synthetic peptides of the present invention to ascertain if these peptides can be used to increase HDL and decrease atherosclerosis.

The mice receive 3 injections per week for either 4-6 or 8-10 weeks. After the completion of the injection, the amount of hardening of the arteries or atherosclerosis is determined in the control injected animals and peptide injected animals to determine if the injections of the synthetic peptide decreased development of atherosclerosis.

The proposed studies test if intraperitoneal infusions of the apoA-I mimetic peptides of the present invention result in decreased aortic atherosclerosis in apoE and LDL receptor knockout mice, two well established mouse models of atherosclerosis.

The mouse is ideal animal specie for the proposed study since well characterized and established mouse models of atherosclerosis are readily available. In particular, apoE and LDL receptor knockout mouse models have been universally employed as animal models for atherosclerosis. Because they are available with a homogenous genetic background, these knockout mice are ideal models for analysis of atherosclerotic lesion formation which is readily impacted by genetic background variability. Additionally, lesion development in apoE and LDL-receptor knockout mice is readily modified by changes in plasma lipoproteins, including HDL, the levels of which are altered by the peptide infusion in this study.

The knockout mouse model is a well established and widely employed animal model for the study of atherosclerosis. Mice are used because of their homogenous genetic background and are ideal models for analysis of atherosclerotic lesion formation which is readily impacted by genetic background variability. Importantly, lesion development in apoE and LDL-receptor knockout mice is highly affected by changes in LDL, HDL and other plasma lipoproteins.

The peptides of the present invention are synthesized according to standard synthetic techniques using tBOC amino acids. The peptides are purified for study by high pressure liquid chromatography. Some peptides are N-acetylated and/or C-terminally amidated.

### Mouse Models of Atherosclerosis

Four to six week old C57B1/6 mice, apoE knockout (JAX 2052) and LDL-receptor knockout (JAX'2207) mice, all in the C57B1/6 background, are obtained from Jackson Laboratories. During the entire study, C57B1/6 and apoE knockout mice are maintained on a regular chow diet (0.02% cholesterol, 3% fat) and LDL-receptor mice are maintained on a Western diet (TD88137; Harlan Teklad; Madison, Wis.—containing 0.20% cholesterol and 21% fat).

### Infusion of Synthetic ApoA-I Mimetic Peptides

Three different infusion studies are conducted.

Aim A (Infusion Study A) to determine the functional half-life of the injection of the synthetic peptide on plasma HDL levels.

In the first study (Infusion Study A), C57B1/6 mice as well as apoE knockout and LDL receptor knockout mice are injected by the intraperitoneal (ip) route or intravenous (iv) route with synthetic peptides of the present invention mimetic (30 mg/kg) on up to four different occasions two weeks apart. To evaluate changes in the plasma lipid and lipoprotein profile associated with injection of the synthetic peptide, blood for lipid analyses is obtained before and at 2, 4, 6, 24 and 48 hours after peptide injection. At the end of the study the animals are sacrificed.

Aim B (Infusion Study B) To determine whether ip injection of the synthetic peptide 3×/wk decreases development of atherosclerosis when assayed 4-5 weeks after initiation of treatment.

Aim C (Infusion Study C) To determine whether ip injection of the synthetic peptide 3×/wk decreases development of atherosclerosis when assayed 8-10 weeks after initiation of treatment.

For infusion studies B and C, mice are injected ip with either placebo or a synthetic peptide of the present invention (30 mg/kg) three times per week for either 4 to 5 weeks (Infusion Study B) or 8 to 10 weeks (Infusion Study C). Blood for lipid and lipoprotein analyses is obtained at the beginning of the study (day 0) and every two weeks after placebo/peptide injection and at the completion of the study. At the completion of the study (4 to 5 weeks for Infusion Study B and 8 to 10 weeks for Infusion Study C), the animals are sacrificed, organs harvested for analyses of cholesterol content and for aortic atherosclerosis.

Statistical methods used to analyze data.

All statistical analyses are conducted in SAS8.2 (SAS Institute, NC). After completion of the atherosclerosis study the mean with standard deviation between the control (C57BI/6) and treated group (apoE knockout or LDL-receptor knockout) is calculated. The differences are tested by t test (PROC TTEST) and p-values less than 0.05 are considered significant. Non-parametric analysis of aortic atherosclerosis are performed by the Mann-Whitney test.

In the first infusion study (Infusion Study A), 5 C57B1/6, 5 apoE knockout and 5 LDL receptor knockout mice are injected (IP) with a synthetic peptide of the present invention and blood is obtained for lipid and lipoprotein analyses. A total of 15 mice are used for Infusion Study A.

A total of 40 mice (20 control-placebo injected and 20 study-peptide injected mice) are utilized in each of the two other infusion studies (Infusion Study B-4 to 5 weeks duration as well as Infusion Study C-8 to 10 weeks duration). Since each infusion study is conducted in two different mouse lines (i.e.: apoE-KO and LDL receptor KO), the total number of mice used for both Infusion Studies B and C is 160.

Total number of mice used for the entire protocol is 175 (five-C57B1/6, eighty five-apoE KO mice and eighty five-LDL receptor KO mice) (These animal numbers take into account an estimated 10% morbidity rate during the course of the study as well as the number of animals previously required to achieve statistical significance during analysis of the non-random distribution aortic lesion pattern that develops in mice).

For Infusion Study A, 5 four to six week old C57B1/6, apoE knockout (JAX 2052) and LDL receptor knockout (JAX 2207) control mice receive ip or iv injections of the synthetic peptide of the present invention for up to four times two weeks apart. The sequence of procedures for this study is as follows:
1) Mice are first anaesthetized by using 1-3% isoflurane by inhalation prior to each ip injection to insure appropriate and complete delivery of placebo/peptide.
2) Mice are injected with either placebo (0.2 ml saline) or apoA-I synthetic peptide (30 mg/kg in 0.2 ml saline) via either the intraperitoneal or intravenous route.
3) In order to evaluate changes in the plasma lipids and lipoproteins in the time-frame between injections, each mouse in this study group is bled from the retro-orbital sinus following administration of a topical anesthesia. before and at 2, 4, 6, 24 and 48 hours after the peptide injection. No more than 300 ul blood is drawn during this 48 hour period.
4) At the end of infusion study A all mice are sacrificed by using Avertin (2.5%, 0.011 ml/gm, ip) or ketamine (80 ug/gm, ip).

For Infusion Studies B and C, 20 control and 20 study four to six weeks old apoE knockout (JAX 2052) and LDL-receptor knockout (JAX 2207) mice receive ip injections of either placebo or the synthetic peptide of the present invention three times per week for a total of either 4-5 weeks (Infusion Study B) or 8-10 weeks (Infusion Study C).
1) Mice are first anaesthetized by using 1-3% isoflurane by inhalation prior to each IP injection to insure appropriate and complete delivery of placebo/peptide.
2) Mice are injected ip with either placebo (0.2 ml saline) or apoA-I synthetic peptide (30 mg/kg in 0.2 ml saline) on Monday, Wednesday and Friday of each study week.
3) To measure plasma lipids and lipoproteins, each mouse in the two study groups is fasted for 4 hours in the morning (7 AM to 11 AM) and then bled from the retro-orbital sinus at the start and end of the infusion study as well as every two weeks after the initial infusion for a total of either 4 weeks (Infusion Study B) or 8 weeks (Infusion Study C). No more than 300 ul blood every two weeks is obtained from each mouse.
4) At the end of Infusion Study B and C all mice are sacrificed by cervical dislocation following isoflurane anesthesia and organs are harvested for analyses of cholesterol as well as aortic atherosclerosis.
   Before intraperitoneal injections, brief inhaled analgesia will be obtained by isoflurane utilizing the E-Z Rodent Anesthesia System in the procedure room. A topical anesthetic (proparacaine) will be applied prior to obtaining blood from the retro-orbital sinus.

The results indicate that the synthetic peptides of the present invention decrease aortic atherosclerosis compared to controls. In one test, peptides SEQ ID NOs; 121 and 624 are tested and are found to decrease aortic atherosclerosis compared to controls.

### Example 16

### Evaluation of Peptide Utility in Rabbits

The isolated peptides of the present invention are examined for anti-inflammatory activity using an in vivo rabbit model of acute proinflammatory changes in the carotid artery. This method is explained in detail by Nicholls et al., (Circulation 2005:111, 1543-1550). Normocholesterolemic rabbits are administered the isolated peptides of the present invention iv in a dose of from 1 to 50 mg per day for 3 days, optionally contained in unilamellar vesicles of phosphatidylcholine, with only unilamellar vesicles of phosphatidylcholine with no peptide, or saline as a control. In one test, SEQ ID NOs; 121 and 624 are administered. On the second day, after administration of the peptides, a periarterial collar is introduced around the carotid artery and filled with saline. Two days later, the rabbits are humanely sacrificed and the carotid arteries are processed and analyzed for the presence of reactive oxygen species, the infiltration of neutrophils, and the expression of adhesion proteins and chemokines. The administration of the peptides of the present invention decrease the presence of reactive oxygen species, the infiltration of neutrophils, and the expression of adhesion proteins and chemokines compared to controls, thereby demonstrating anti-inflammatory activity in vivo, which can help retard the atherogenic process.

### Example 17

### Evaluation of Peptide Utility to Promote Reverse Cholesterol Transport In Vivo

The isolated peptides of the present invention are examined for the ability to release cholesterol in mice using the method described by Zhang et al., (Circulation. 2003; 108: 661-663). Macrophages (J774 cells) are loaded with tritiated cholesterol in vitro and injected ip into mice. These mice are administered isolated peptides of the present invention, iv, at a dose of from 1 ug to 1 mg, or saline as a control. In one test, SEQ ID NOs; 121 and 624 are administered. The peptides are administered either in saline as a vehicle or in lipid vesicles, such as vesicles of phosphatidylcholine. The mice receiving the peptides of the present invention demonstrate increased levels of tritiated cholesterol in the liver, plasma and feces, than mice receiving saline. The results demonstrate that the peptides of the present invention stimulate reverse cholesterol transport from macrophages to the liver and feces.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims. It will be apparent that the precise details of the constructs, compositions, and methods described herein may be varied or modified without departing from the spirit of the described invention. We claim all such modifications and variations that fall within the scope and spirit of the claims below.

1. An isolated peptide of formula I comprising:
(A-B-C)ₙ, I
wherein
A comprises helix 5 of ApoA-I, helix 6 of ApoA-I, or a modified form of helix 8 of ApoA-I;
C comprises helix 8 of ApoA-I;
B comprises a linking group between A and C; and,
n is an integer from 1 to 10.

2. The isolated peptide of claim 1, wherein,
A is SEQ ID NO: 1 Gly Glu Glu Met Arg Asp Arg Ala Arg Ala His Val Asp Ala Leu Arg Thr His, SEQ ID NO: 2 His Thr Arg Leu Ala Asp Val His Ala Arg Ala Arg Asp Arg Met Glu Glu Gly, SEQ ID NO: 3 Ser Asp Glu Leu Arg Gln Arg Leu Ala Ala Arg Leu Glu Ala Leu Lys Glu Asn, SEQ ID NO: 4 Asn Glu Lys Leu Ala Glu Leu Arg Ala Ala Leu Arg Gln Arg Leu Glu Asp Ser, SEQ ID NO: 5 Leu Glu Ser Ala Lys Val Ser Ala Leu Ser Ala Leu Glu Glu Ala Thr Lys Lys, or SEQ ID NO: 6 Lys Lys Thr Ala Glu Glu Leu Ala Ser Leu Ala Ser Val Lys Ala Ser Glu Leu, or a conservative substitution thereof,
B is Pro, SEQ ID NO: 7 Lys Leu Ser Pro Leu, SEQ ID NO: 8 Leu Ser Pro Leu, or SEQ ID NO: 9 Ser Pro Leu, SEQ ID NO: 12 Leu Pro Ser, SEQ ID NO: 11 Leu Pro Ser Leu, SEQ ID NO: 10 Leu Pro Ser Leu Lys, or a conservative substitution thereof,
C is SEQ ID NO: 13 Leu Glu Ser Phe Lys Val Ser Phe Leu Ser Ala Leu Glu Glu Tyr Thr Lys Lys, SEQ ID NO: 14 Lys Lys Thr Tyr Glu Glu Leu Ala Ser Leu Phe Ser Val Lys Phe Ser Glu Leu, or a conservative substitution thereof, and
n is 1.

3. The isolated peptide of claim 1, wherein a N-terminal amino acid is acetylated and a C-terminal amino acid is amidated.

4. The isolated peptide of claim 1, further comprising one or more of D, E, F, and W, to form subgeneric formula II,
D-E-(A-B-C)ₙ-F-W, II
wherein
D is absent or present and is selected from the group consisting of SEQ ID NO: 15 Pro Arg Gly Gly Ser Val Leu Val Thr, SEQ ID NO: 16 Thr Val Leu Val Ser Gly Gly Arg Pro, or multiples, variations or conservative substitutions thereof;
E is absent or present and is Pro, SEQ ID NO: 10 Leu Pro Ser Leu Lys, SEQ ID NO: 7 Lys Leu Ser Pro Leu, or a conservative substitution thereof, provided E is present if D is present;
F is absent or present and is Pro, SEQ ID NO: 19 Ala Leu Ser Pro Leu, SEQ ID NO: 20 Leu Pro Ser Leu Ala or a conservative substitution thereof, provided F is present if W is present; and,
W is absent or present and is a peptide selected from the group consisting of SEQ ID NO: 21 Trp Arg Trp Trp Trp Trp, SEQ ID NO: 22 Trp Trp Trp Trp Arg Trp, or multiples, variations or conservative substitutions thereof.

5. The isolated peptide of claim 1, further comprising one or more of G and H, to form subgeneric formula IV,
G-(A-B-C)ₙ-H, IV
wherein
G is absent or present and is SEQ ID NO: 9 Ser Pro Leu, SEQ ID NO: 12 Leu Pro Ser or a variation or a conservative substitution thereof; and,
H is absent or present and is SEQ ID NO: Leu Asn Thr Gln, Gln Thr, SEQ ID NO: Gln Thr Asn or a variation or a conservative substitution thereof.

6. The isolated peptide of claim 1, further comprising one or more of D, E, F, W, G and H to form subgeneric formula V,
D-E-G-(A-B-C)ₙ-H-F-W, V
wherein
D is absent or present and is a peptide selected from the group consisting of SEQ ID NO: 15 Pro Arg Gly Gly Ser Val Leu Val Thr, SEQ ID NO: 16 Thr Val Leu Val Ser Gly Gly Arg Pro, or multiples, variations or conservative substitutions thereof;
E is absent or present and is Pro, SEQ ID NO: 10 Leu Pro Ser Leu Lys, SEQ ID NO: 7 Lys Leu Ser Pro Leu, or a conservative substitution thereof, provided E is present if D is present;
F is absent or present and is Pro, SEQ ID NO: 19 Ala Leu Ser Pro Leu, SEQ ID NO: Leu Pro Ser Leu Ala or a conservative substitution thereof, provided that F is present when W is present;
W is absent or present and is a peptide selected from the group consisting of SEQ ID NO: 21 Trp Arg Trp Trp Trp Trp, SEQ ID NO: 22 Trp Trp Trp Trp Arg Trp, or multiples, variations or conservative substitutions thereof;
G is absent or present and is SEQ ID NO: 9 Ser Pro Leu, SEQ ID NO: 12 Leu Pro Ser or a variation or a conservative substitution thereof, provided G is present when D is absent;
H is absent or present and is SEQ ID NO: 23 Leu Asn Thr Gln, SEQ ID NO: 24 Gln Thr Asn Leu or a variation or a conservative substitution thereof, provided H is present when W is absent.

7. An isolated peptide of formula VI
D-I-W, VI
wherein
D is absent or present and is a peptide selected from the group consisting of SEQ ID NO: 15 Pro Arg Gly Gly Ser Val Leu Val Thr, SEQ ID NO: 16 Thr Val Leu Val Ser Gly Gly Arg Pro, or multiples, variations or conservative substitutions thereof;
I is a group linking D and W and is GABA, Pro, SEQ ID NO: 7 Lys Leu Ser Pro Leu, SEQ ID NO: 25 Leu Lys Leu Ser Pro Leu, SEQ ID NO: 26 Leu Pro Ser Leu Lys Leu or multiples, variations, combinations thereof or conservative substitutions thereof;
W is absent or present and is a peptide selected from the group consisting of SEQ ID NO: 21 Trp Arg Trp Trp Trp Trp, SEQ ID NO: 22 Trp Trp Trp Trp Arg Trp, or multiples, variations or conservative substitutions thereof.

8. An isolated peptide of formula VII comprising:
(D-R-S-W-T-N-O-(Xₙ-Y_{z}-Zₘ)ₛ-O′-N′-T′-W′-S′-R′-D′)ᵣ VII
wherein:
n is an integer from 1 to 3, m is an integer from 1 to 3, r is an integer from 1 to 10, and s is an integer from 1 to 5;
z is an integer from 1 to 13 and is number of times Y may be present in Xₙ-Y_{z}-Zₘ when n or m in Xₙ-Y_{z}-Zₘ are more than 1 or when s is more than 1,
D or D′ is individually absent or present and is SEQ ID NO: 15 Pro Arg Gly Gly Ser Val Leu Val Thr, SEQ ID NO: 16 Thr Val Leu Val Ser Gly Gly Arg Pro, or multiples, variations or conservative substitutions thereof;
R or R′ is individually absent or present and is comprised of at least one GABA, or one or more neutral amino acids;
S or S′ is individually absent or present and is comprised of from 1 to 10 amino acid residues, wherein the amino acid residues are proline, alanine, leucine, lysine, serine, glycine, polymers thereof or combinations thereof, or is an alkyl group (CH₂)ₙ, wherein n is an integer from 1-20;
W or W′ is individually absent or present and is selected from the group consisting of SEQ ID NO: 21 Trp Arg Trp Trp Trp Trp, SEQ ID NO: 22 Trp Trp Trp Trp Arg Trp, or multiples, variations or conservative substitutions thereof;
T or T′ is individually absent or present and is comprised of at least one GABA, or one or more neutral amino acids or polymers thereof and combinations thereof;
N or N′ is individually absent or present and is comprised of from 1 to 10 amino acid residues, wherein the amino acid residues are proline, alanine, leucine, serine, glycine, polymers thereof or combinations (co-polymers) thereof, or is an alkyl group (CH₂)ₙ, wherein n is an integer from 1-20;
O or O′ is individually absent or present and is comprised of at least one GABA, or one or more neutral amino acids, or combinations thereof;
X is comprised of from 5 to 25 amino acid residues, provided an amphipathic alpha helix is obtained;
Y is absent or present and is comprised of amino acids of from 1 to 10 amino acid residues, wherein the amino acid residues are proline, alanine, leucine, serine, glycine, lysine, polymers thereof or combinations thereof, or is an alkyl group (CH₂)ₙ, wherein n is an integer from 1-20; and,
Z is comprised of from 5 to 25 amino acids residues, provided an amphipathic alpha helix is obtained.

9. The isolated peptide of claim 1, further comprising a label.

10. A pharmaceutical composition comprising the isolated peptide of claim 1 and a pharmaceutically acceptable carrier.

11. A pharmaceutical composition comprising the isolated peptide of claim 4 and a pharmaceutically acceptable carrier.

12. A pharmaceutical composition comprising the isolated peptide of claim 5 and a pharmaceutically acceptable carrier.

13. A pharmaceutical composition comprising the isolated peptide of claim 6 and a pharmaceutically acceptable carrier.

14. A pharmaceutical composition comprising the isolated peptide of claim 7 and a pharmaceutically acceptable carrier.

15. A pharmaceutical composition comprising the isolated peptide of claim 8 and a pharmaceutically acceptable carrier.

16. A method of treating disease in an animal or a human comprising administering to the animal or the human the pharmaceutical composition of claim 10.

17. A method of treating disease in an animal or a human comprising administering to the animal or the human the pharmaceutical composition of claim 11.

18. A method of treating disease in an animal or a human comprising administering to the animal or the human the pharmaceutical composition of claim 12.

19. A method of treating disease in an animal or a human comprising administering to the animal or the human the pharmaceutical composition of claim 13.

20. A method of treating disease in an animal or a human comprising administering to the animal or the human the pharmaceutical composition of claim 14.

21. A method of treating disease in an animal or a human comprising administering to the animal or the human the pharmaceutical composition of claim 15.

22. A method of treating or inhibiting a dyslipidemic disorder or a vascular disorder in an animal or a human, comprising administering to the animal or the human a therapeutically effective amount of the pharmaceutical composition of claim 10.

23. A method of treating or inhibiting a dyslipidemic disorder or a vascular disorder in an animal or a human, comprising administering to the animal or the human a therapeutically effective amount of the pharmaceutical composition of claim 11.

24. A method of treating or inhibiting a dyslipidemic disorder or a vascular disorder in an animal or a human, comprising administering to the animal or the human a therapeutically effective amount of the pharmaceutical composition of claim 12.

25. A method of treating or inhibiting a dyslipidemic disorder or a vascular disorder in an animal or a human, comprising administering to the animal or the human a therapeutically effective amount of the pharmaceutical composition of claim 13.

26. A method of treating or inhibiting a dyslipidemic disorder or a vascular disorder in an animal or a human, comprising administering to the animal or the human a therapeutically effective amount of the pharmaceutical composition of claim 14.

27. A method of treating or inhibiting a dyslipidemic disorder or a vascular disorder in an animal or a human, comprising administering to the animal or the human a therapeutically effective amount of the pharmaceutical composition of claim 15.

28. A method of visualizing plaque comprising:
administering the labeled peptide of claim 9 in an acceptable carrier to a vascular system of an animal or a human; and,
detecting the labeled peptide bound to the plaque within the vascular system of the animal or the human.
